(19) United States
(12) Patent Application Publication
(10) Pub. No.: US 2025/0283056 A1
(43) Pub. Date: Sep. 11, 2025

(54) MODIFIED O-METHYLTRANSFERASES USEFUL FOR PRODUCTION OF O-METHYLATED PHENOLIC NATURAL PRODUCTS

(71) Applicant: The University of Hong Kong, Hong Kong (CN)

(72) Inventors: Sze Chung Clive Lo, Hong Kong (CN); Cheuk Woon Lui, Hong Kong (CN); Quan Hao, Dongguan (CN); Kah Chee Pow, Penang (MY)

(21) Appl. No.: 18/600,046

(22) Filed: Mar. 8, 2024

(51) Int. Cl. C12N 9/10; C07C 37/06; C07C 41/01; C07C 51/377; C12N 15/82; C12P 7/22; C12P 7/42

(52) U.S. Cl. CPC C12N 9/1007; C07C 37/06; C07C 41/01; C07C 51/377; C12N 15/8243; C12P 7/22; C12P 7/42

(57) ABSTRACT

Disclosed are modified Type 1 O-methyltransferases (OMTs) and methods of using such modified OMTs. In some forms of the modified OMTs, the amino acid corresponding to Phe337 of Sorghum bicolor stilbene OMT (SbSOMT) is substituted with a polar amino acid. In some forms of the modified OMTs, the amino acid corresponding to Asn323 of Sorghum bicolor caffeic acid OMT (SbCOMT) is substituted with a hydrophobic amino acid. In some forms of the modified OMTs, the amino acid corresponding to Ile144 of SbSOMT is substituted with a polar amino acid. In some forms of the modified OMTs, the amino acid corresponding to Asn128 of SbCOMT is substituted with a hydrophobic amino acid.

### REFERENCE TO SEQUENCE LISTING

The Sequence Listing XML submitted as a file named “UHK_01454_US_ST26.xml,” created on Mar. 6, 2024, and having a size of 109,775 bytes is hereby incorporated by reference pursuant to 37 C.F.R. § 1.834(c)(1).

### FIELD OF THE INVENTION

The disclosed invention is generally in the field of O-methylation of natural products and specifically in the area of modified O-methyltransferases that methylate stilbenes, and related phenylpropanoids.

### BACKGROUND OF THE INVENTION

Stilbenes are specialized metabolites sporadically distributed in the plant kingdom. Grape (Vitis vinifera, dicot), peanut (Arachis hypogaea, dicot), Scots pine (Pinus sylvestris, gymnosperm), Sorghum (Sorghum bicolor, monocot) and sugarcane (Saccharum spp., monocot) are iconic species producing various stilbene compounds upon (a)biotic challenges¹⁻⁵. They serve to maintain reactive oxygen species homeostasis and/or protect against microbial attacks³⁻⁶⁻⁸. Recently, stilbenes have emerged as prominent candidates for nutritional and pharmaceutical studies due to their anti-aging, anti-neurodegeneration, anti-diabetes and chemo-prevention properties⁹⁻¹¹. Over 200 stilbene-related clinical trials (internet site clinicaltrials.gov/) have been launched and stilbenes like resveratrol and pterostilbene are commercialized as dietary supplements. Despite the heightened interest in this minor, yet unique, class of phenylpropanoids as pharmaceuticals and nutraceuticals, in planta stilbene biosynthesis remains understudied, especially the downstream derivatization steps such as O-methylations and prenylations. Elucidation of the underlying enzymology would expedite bioengineering attempts to fortify crops with more or novel stilbenes.

O-Methyltransferases (OMTs) are indispensable in a diversity of metabolic pathways and hence biophysiological processes. For instance, Sorghum SbOMT3 catalyzes the biosynthesis of sorgoleones which are allelochemicals exuded by root hairs to suppress competing weeds¹². Two maize OMTs regioselectively methylate flavonoid phytoalexins which inhibit Fusarium spp. infection¹³. The degree of O-methylations of anthocyanidin pigments affects floral and fruit colors in flowering plants¹⁴⁻¹⁵. In Arabidopsis (Arabidopsis thaliana), disruption of a tapetum-specific OMT (AtTSM1) which methylates polyamine conjugates adversely affects silique development and seed setting¹⁶. Additionally, the degree of phenylpropanoid O-methylation determines lignin composition in tracheophytes¹⁷. For example, disruption of caffeic acid O-methyltransferase (COMT) and/or caffeoyl-CoA O-methyltransferase (CCoAOMT) substantially alters lignin content and composition, potentially affecting plant growth and development¹⁸⁻²⁰. Biochemical and structural characterization of these phenylpropanoid OMTs have provided insights into their substrate enzyme interactions and catalytic mechanisms²¹⁻²⁴, hence facilitating genetic manipulations of lignin composition in biomass crop^{25, 26}.

Phylogenetically-unrelated plant species have independently recruited specific OMTs for stilbene O-methylation. In response to environmental stresses, grapevine resveratrol OMT (VvROMT) methylates resveratrol to pterostilbene whereas Scots pine pinosylvin OMT (PsPMT2) methylates pinosylvin to pinosylvin monomethyl ether^{27, 28}. In addition, a root hair-specific sorgoleone O-methyltransferase from Sorghum, SbOMT3, methylates resveratrol to pterostilbene in vitro or in transgenic models, albeit at poor efficiency^{12, 29}. Synthetic biology approaches have been initiated to generate O-methylated stilbenes of pharmaceutical significance such as pterostilbene and 3′-hydroxypterostilbene^{30, 31}, which are critically acclaimed for their potent cancer-chemopreventive³² and neuroprotective properties³³. In fact, their pharmacological properties are superior to the non-O-methylated analogs³⁴³⁵. However, the lack of stilbene O-methyltransferase (SOMT) structural data greatly limits our understanding of their distinctive substrate specificities, regioselectivities and catalytic efficiencies, impeding bioengineering efforts in the production of high-value and/or novel O-methylated stilbenes.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### BRIEF SUMMARY OF THE INVENTION

Disclosed are modified Type 1 O-methyltransferases (OMTs) and methods of using such modified OMTs. In some forms of the modified OMTs, the amino acid corresponding to Phe337 of Sorghum bicolor stilbene OMT (SbSOMT) is substituted with a polar amino acid. In some forms of the modified OMTs, the amino acid corresponding to Asn323 of Sorghum bicolor caffeic acid OMT (SbCOMT) is substituted with a hydrophobic amino acid. In some forms of the modified OMTs, the amino acid corresponding to Ile144 of SbSOMT is substituted with a polar amino acid. In some forms of the modified OMTs, the amino acid corresponding to Asn128 of SbCOMT is substituted with a hydrophobic amino acid.

In some forms, the substituting polar amino acid is independently Asn, Gln, Thr, or Ser. In some forms, the substituting polar amino acid is Asn. In some forms, the substituting hydrophobic amino acid is independently Phe, Leu, Trp, Tyr, Ile, Met, Val, or Ala. In some forms, the substituting hydrophobic amino acid is Phe.

In some forms, the modified OMT is a modified form of Arabidopsis thaliana AtOMT1, Carthamus tinctorius CtCAldOMT, Catharanthus roseus CrCOMT, Hordeum vulgare HvCOMT, Medicago sativa MsCOMT, Medicago sativa MsIOMT, Oryza sativa OsCAldOMT1, Oryza sativa OsNOMT, Panicum virgatum PvCOMT, Pinus sylvestris PsPMT2, Pinus taeda PtAEOMT, Populus tremuloides PtCOMT, Rosa hybrid RhOOMT, Saccharum spontaneum SsSTS, Saccharum spontaneum SsCOMT, Solanum lycopersicum SICOMT, Sorghum bicolor SbCOMT, Sorghum bicolor SbNOMT, Sorghum bicolor SbOMT1, Sorghum bicolor SbOMT3, Sorghum bicolor SbOMT4, Sorghum bicolor SbSOMT, Sorghum bicolor SbSTS1, Sorghum halepense ShOMT1, Sorghum halepense ShSOMT, Triticum aestivum TaCOMT1, Triticum aestivum TaCOMT2, Vitis vinifera VvCOMT, Vitis vinifera VvROMT, Zea mays ZmCOMT, Zea mays ZmNOMT, Medicago truncatula MtIOMT, Medicago truncatula MtIOMT3, Streptomyces carzinostaticus ScNOMT, Loium perenne LpCOMT, Clarkia breweri CbIEOMT, Planctopirus limnophila PlOMT, Thalictrum flavum TfNOMT, Kitagawia praeruptora KpBOMT, Papaver somniferum PsSOMT, or Fragaria ananassa FaOMT.

In some forms, the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93. In some forms, the modified OMT has at least 70% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT has at least 75% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT has at least 80% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT has at least 85% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT has at least 90% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT has at least 95% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Phe337 of Sorghum bicolor stilbene OMT (SbSOMT) substituted with a polar amino acid.

In some forms, the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Asn323 of Sorghum bicolor caffeic acid OMT (SbCOMT) substituted with a hydrophobic amino acid.

In some forms, the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Ile144 of SbSOMT substituted with a polar amino acid.

In some forms, the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Asn128 of SbCOMT substituted with a hydrophobic amino acid.

In some forms, the modified OMT is further modified to improve or alter the production of an O-methylated stilbenoid or hydroxycinnamic acid by the modified OMT.

Also disclosed are solutions including a modified OMT as disclosed. In some forms, the solution is composed to allow production of one or more O-methylated forms of stilbenoid or hydroxycinnamic acid from a substrate for the modified OMT under suitable conditions. Also disclosed are plants that express a modified OMT as disclosed.

Also disclosed are methods of producing an O-methylated stilbenoid or hydroxycinnamic acid. In some forms, the method involves bringing into contact, in a solution, a modified OMT as disclosed, a substrate for the modified OMT, and S-adenosylmethionine. In general, the O-methylated stilbenoid or hydroxycinnamic acid is produced by incubating the solution under suitable conditions.

In some forms, the method involves growing that express a modified OMT as disclosed and extracting the O-methylated stilbenoid or hydroxycinnamic acid from the grown plant.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

FIGS. 1A-1F are schematic and plots showing biosynthesis of stilbenes and flavonoids in Sorghum and stilbene profiles of Colletotrichum sublineola-infected Sorghum mesocotyls. FIG. 1A illustrate biosynthetic pathways in stilbene and flavonoid in Sorghum. Newly formed methoxy groups are enlarged. STS, stilbene synthase; SOMT, stilbene O-methyltransferase; COMT: caffeic acid O-methyltransferase; CHS, chalcone synthase; CHI; chalcone isomerase; FNR, flavanone 4-reductase; DFR, dihydroflavonol 4-reductase; FNSII, flavone synthase II; Sb, Sorghum bicolor. R=H, OH or OCH₃. FIG. 1B is a plot showing distribution of stilbene aglycones in C. sublineola-infected mesocotyls of Sorghum genotypes SC748-5 (resistant) and BTx623 (susceptible) 96 h after treatment. Values refer to means±SD (n=3). FIGS. 1C-1F are plots showing HPLC-QTOF-HRMS detection of resveratrol (FIG. 1C), piceid (FIG. 1D), pinostilbene (FIG. 1E) and pterostilbene (FIG. 1F) in C. sublineola-infected mesocotyls of Sorghum genotypes SC748-5 (resistant) and BTx623 (susceptible). inf, infected; ctrl, control; cps, counts per second.

FIGS. 2A-2D are plots showing catalytic activities and phylogeny of SbSOMT and SbCOMT. FIG. 2A is a pair of plot showing formation of pinostilbene and pterostilbene from resveratrol by SbSOMT and SbCOMT after two-hour incubation with 100 μM resveratrol. Values refer to means±SD (n=3). n.d.: not detected. Dots represent individual data points. FIG. 2B is plot showing formation of ferulic acid from caffeic acid by SbSOMT and SbCOMT after two-hour incubation with 100 μM phenylpropanoid substrates. Values refer to means±SD (n=3). n.d.: not detected. Dots represent individual data points. FIG. 2C is a plot showing formation of sinapic acid from 5-hydroxyferulic acid by SbSOMT and SbCOMT after two-hour incubation. Values refer to means±SD (n=3). n.d.: not detected. FIG. 2D demonstrates phylogenetic analysis of SbSOMT and SbCOMT. The unrooted phylogenetic tree was constructed by maximum likelihood using MEGA X⁹⁰. Bootstrapping with 1,000 replications was carried out. Bona fide stilbene O-methyltransferases from gymnosperm²⁷, dicot²⁸ and monocot (this study) are highlighted. Sorghum OMTs are bolded. Scale bar denotes 0.2 amino acid substitution per site.

FIGS. 3A-3E are plots showing genotypes, phenotypes and metabolite analysis of Sorghum sbsomt mutants. FIG. 3A is a schematic showing genotypes of Sorghum sbsomt mutants. Nucleotides corresponding to the protospacer adjacent motif (PAM) are bolded. CRISPR/Cas9-mediated mutations in sbsomt mutant lines are underlined. Sequences are SEQ ID NO:94 (WT, exon 1), SEQ ID NO:95 (WT, exon 2), SEQ ID NO:96 (sbsomt-a, exon 1), SEQ ID NO:97 (sbsomt-a, exon 2), SEQ ID NO:98 (sbsomt-b1, exon 1), SEQ ID NO:99 (sbsomt-b1, exon 2), SEQ ID NO:100 (sbsomt-b2, exon 1), and SEQ ID NO:101 (sbsomt-b2, exon 2). FIGS. 3B-3E are plots showing HPLC-QTOF-HRMS detection of resveratrol (FIG. 3B), piceid (FIG. 3C), pinostilbene (FIG. 3D) and pterostilbene (FIG. 3E) in C. sublineola-infected mesocotyls of Sorghum sbsomt mutants after 96 h. inf, infected; ctrl, control; cps, counts per second.

FIGS. 4A-4F demonstrate regioselective O-methylation of piceatannol and three-dimensional structure of SbSOMT and SbCOMT. FIG. 4A show structural comparison between resveratrol, caffeic acid, and piceatannol. Structural similarities are colored accordingly (pink: resveratrol-piceatannol; blue: caffeic acid-piceatannol). FIG. 4B show schematic of regioselective O-methylation of piceatannol catalyzed by SbSOMT (in pink) and SbCOMT (in blue). FIG. 4C Formation of different A- and B-ring O-methylated products from piceatannol by SbSOMT and SbCOMT after two-hour reaction with 100 μM stilbenes. Values refer to means±SD (n=3). n.d.: not detected. Dots represent individual data points. FIG. 4D represents a global structure of dimeric SbSOMT-resveratrol-β-NAD ternary complex solved at 1.72 Å resolution. In which, the two protomers are colored in white and grey, respectively. FIG. 4E highlights the functional domains, namely dimerization domain, substrate binding pocket and SAM-binding domain of a SbSOMT protomer and position of co-crystallized resveratrol (in black) bound to the substrate binding pocket of SbSOMT. FIG. 4F superimposed SbSOMT (in white) and SbCOMT (in grey; retrieved from PDB: 4PGH²¹) to show the high structural similarity between SbSOMT and SbCOMT (RMSD=2.322 Å) and the elongated threonine-rich loop (Val103-Cys114) unique to SbSOMT.

FIGS. 5A-5F are structural models showing interactions between stilbenes and SbSOMT catalytic residues, and docking positions of stilbenes with SbSOMT and SbCOMT. FIG. 5A represents a close-up view of SbSOMT substrate binding pocket revealed resveratrol conformation and resveratrol-interacting residues and water. Dashed line indicates hydrogen bond formation with the distance labelled. FIGS. 5B-5D are structural models that illustrate close-up view on interactions between SbSOMT catalytic residues (His282, Asp283, Glu310 and Glu342) and resveratrol (FIG. 5B), pinostilbene (FIG. 5C), and pterostilbene (FIG. 5D). Dashed line indicates hydrogen bond formation with the distance labelled. FIG. 5E shows the docking conformation of piceatannol ligand (in black) in SbSOMT (left) and SbCOMT (right) along with the surrounding residues-of-interest and calculated binding energy. Dashed line indicates hydrogen bond formation with the distance labelled. FIG. 5F shows the two docking conformations of resveratrol in SbCOMT along with the surrounding residues-of-interest and calculated binding energy. The carbon backbone of non-productive conformation is coloured in black, and the productive conformation is coloured in grey, respectively. Dashed line indicates hydrogen bond formation with the distance labelled.

FIGS. 6A-6D are plots demonstrating validation of catalytic residues and key substrate-orientating residues of SbSOMT via site-directed mutagenesis. FIGS. 6A-6B are a pair of plots showing formation of pinostilbene (FIG. 6A) and pterostilbene (FIG. 6B) by catalytic residue mutant proteins over a one-hour incubation period. Data are expressed as yields relative to that of wild-type SbSOMT protein (WT). FIGS. 6C-6D are a pair of plots showing formation of pinostilbene (FIG. 6C) and pterostilbene (FIG. 6D) by key substrate-orientating residue mutant proteins over a one-hour incubation period. Data are expressed as yields relative to that of wild-type SbSOMT protein (WT). Values refer to means±SD (n=3). Asterisks indicate significant differences between mutant protein and WT (Student's t-test, ***: p<0.001). Tricin was used as an internal standard for quantitation. Note that pterostilbene represents the major product for SbSOMT(WT)-resveratrol reactions at the 2-hour end point (FIG. 2A). n.d.: not detected. Dots represent individual data points.

FIGS. 7A-7C illustrate biosynthesis of stilbenes in wild sugarcane, stilbene profiles of mechanically-wounded wild sugarcane stalks, and in vitro enzyme activities of SsCOMT. FIG. 7A is a schematic showing proposed stilbene pathways in wild sugarcane. Newly formed methoxy groups are colored and bolded. STS, stilbene synthase; COMT: caffeic acid O-methyltransferase; Ss, Saccharum spontaneum. FIG. 7B is a plot showing distribution of stilbene aglycones in mechanically-wounded wild sugarcane stalks 120 h after treatment. Values refer to means±SD (n=3). FIG. 7C is a plot showing formation of ferulic acid, sinapic acid, isorhapontigenin, pinostilbene, and pterostilbene from caffeic acid, 5-hydroxyferulic acid, piceatannol, and resveratrol respectively, by SsCOMT, after two-hour incubation with 100 μM substrates. Tricin was used as an internal standard for quantitation. Values refer to means±SD (n=3).

FIGS. 8A-8D are plots showing expression analysis of pterostilbene biosynthesis related candidate genes in Sorghum. FIG. 8A shows analysis of in silico gene expression. RNA-sequencing datasets derived from Bipolaris sorghicola-infected Sorghum leaves and Colletotrichum sublineola-infected Sorghum mesocotyls were obtained from literature^{40, 41}. FIGS. 8B-8D are plots showing quantitative RT-PCR gene expression analysis of SbSTS1 (FIG. 8B), SbOMT4 (FIG. 8C) and SbSOMT (FIG. 8D). Expression levels are expressed relative to Sorghum bicolor Eukaryotic Initiation Factor 4A-1 (SbEIF4α)⁶⁸. Values refer to means±SD (n=3). inf, infected; ctrl, control.

FIGS. 9A-9C demonstrate initial screening of catalytic activities of Sorghum OMTs. FIG. 9A represents chemical structures showing in vitro O-methylation of resveratrol by Sorghum OMTs. FIGS. 9B-9C are a pair of plots showing HPLC-QTOF-HRMS detection of pinostilbene (FIG. 9B), and pterostilbene (FIG. 9C) in Sorghum OMTs catalyzed reaction using resveratrol as a substrate. cps, counts per second; NC, negative control (reaction without enzyme).

FIGS. 10A-10D demonstrate co-overexpression of SbSTS1 and SbSOMT in Nicotiana benthamiana leaves. FIG. 10A is a schematic showing in planta generation of pterostilbene by Sorghum SbSTS1 and SbSOMT. FIGS. 10B-10D are plots showing HPLC-QTOF-HRMS detection of resveratrol (FIG. 10B), pinostilbene (FIG. 10C), and pterostilbene (FIG. 10D) in Nicotiana benthamiana leaves transiently expressing SbSTS1 or co-expressing SbSTS1 and SbSOMT. Cps, counts per second.

FIG. 11A-11F are plots showing genotyping of Sorghum sbsomt mutants. FIGS. 11A-11F are representative chromatographs of direct sequencing of the target sites in the sbsomt-a (FIGS. 11A-11B), sbsomt-b1 (FIGS. 11C-11D), and sbsomt-b2 (FIGS. 11E-11F) mutant lines (T₁ generation) are shown. CRISPR/Cas9-mediated mutations in sbsomt mutant lines are underlined. Bold, protospacer adjacent motif (PAM) site. Sequences are SEQ ID NO:94 (reference, target 1), SEQ ID NO:95 (reference, target 2), SEQ ID NO:96 (sbsomt-a, target 1), SEQ ID NO:97 (sbsomt-a, target 2), SEQ ID NO:98 (sbsomt-b1, target 1), SEQ ID NO:99 (sbsomt-b1, target 2), SEQ ID NO:100 (sbsomt-b2, target 1), and SEQ ID NO:101 (sbsomt-b2, target 2).

FIG. 12 demonstrates predicted effects of mutations in SbSOMT in Sorghum sbsomt mutant lines. The amino acid sequences of wild-type and mutant SbSOMT were aligned using Clustal Omega⁸⁸. First premature stop codons (*) are underlined and bolded. The catalytic residues of Sorghum SbSOMT are underlined and bolded. Amino acid residues contributing to the hydrophobic interactions with stilbene substrates are labelled with t above the corresponding residues. Sequences are SEQ ID NO:1 (WT), SEQ ID NO:102 (sbsomt-a), SEQ ID NO:103 (sbsomt-b1), and SEQ ID NO:104 (sbsomt-b2).

FIG. 13 demonstrates multiple sequence alignment of bonafide stilbene O-methyltransferases (SOMTs). The amino acid sequence of SbSOMT, VvROMT and PsPMT2 were aligned using Clustal Omega⁸⁸. The catalytic residues of Sorghum SbSOMT are underlined and bolded, while those of other SOMTs are only underlined. Amino acid residues contributing to the hydrophobic interactions with stilbene substrates are labelled with t above the corresponding residue. Fully conserved amino acid residues across all three SOMTs are indicated by asterisks; those with strongly similar properties are indicated with colons; those with weakly similar properties are indicated with periods. Sequences are SEQ ID NO:1 (SbSOMT), SEQ ID NO:27 (VvROMT), and SEQ ID NO:14 (PsPMT2).

FIG. 14 is a schematic showing phylogeny of wild sugarcane OMTs. Phylogenetic analysis of wild sugarcane OMTs. The unrooted phylogenetic tree was constructed by maximum likelihood using MEGA X⁹⁰. Bootstrapping with 1,000 replications was carried out. Scale bar denotes 0.5 amino acid substitution per site. The sequences of the noted genes are hereby incorporated by reference.

FIGS. 15A-15X are titration thermograms and fitting curves of conducted ITCs. Each set of thermogram (above) and fitting curve (below) represents performed ITC of different combinations of protein (in cell) and ligand (in syringe) as titled.

FIGS. 16A-16C are structures obtained in this study. FIG. 16A is superimposed SbSOMT-resveratrol complex with two SbSOMT-stilbene-β-NAD complexes obtained in this study towards SbSOMT-resveratrol-β-NAD (top left). SbSOMT-resveratrol complex (top right; RMSD=0.468 Å), SbSOMT-pinostilbene-β-NAD, (bottom left; RMSD=0.995 Å) and SbSOMT-pterostilbene-β-NAD (bottom right; RMSD=0.730 Å) complexes all shares high structural similarity towards SbSOMT-resveratrol-β-NAD. FIG. 16B is a close-up view focusing the conformations of superimposed stilbene derivatives (in black) and correspondent surrounding residues. Leu28 (in grey) is constituted in adjacent protomer. FIG. 16C illustrate the FO-FC electron density omit maps of resveratrol and β-NAD ligands in SbSOMT-resveratrol-β-NAD ternary complex contoured at 3σ (mesh) and −3σ (blob).

FIG. 17 is a schematic showing reaction mechanism of consecutive stilbene O-methylations mediated by SbSOMT. Schematic of SbSOMT O-methylation cycle exemplified with S-adenosyl methionine (SAM, donor) and resveratrol (acceptor)^{21, 22, 43, 44}. Upon binding of SAM and resveratrol (Step I), the surrounding electrostatic interactions allow His282 N_{ε} to deprotonate 3-OH of resveratrol. Step II, deprotonated O⁻ catalyzes SN₂ attack towards methyl group of SAM sulfonium ylide to produce pinostilbene and S-adenosyl-homocysteine (SAH). Step III, the His282 protonated imidazole is regenerated by solvent and returns to the neutral state. Step IV, the SAH and pinostilbene are dissociated from SbSOMT.

FIGS. 18A-18C are plots showing stilbene profiles of mechanically-wounded wild sugarcane stalks. FIGS. 18A-18C demonstrate HPLC-QTOF-HRMS detection of resveratrol (FIG. 18A), piceatannol (FIG. 18B), and isorhapontigenin (FIG. 18C) in mechanically-wounded wild sugarcane stalks 120 h after treatment. Cps, counts per second.

FIGS. 19A-19B are a pair of plots showing tentative identification of piceatannol-hexoside from mechanically wounded wild sugarcane stalks. FIG. 19A demonstrates HPLC-QTOF-HRMS detection of putative piceatannol-hexoside. FIG. 19B demonstrates MS² fragmentation pattern of putative piceatannol-hexoside. A neutral loss of 162 Da, potentially corresponding to an anhydrohexose unit, was observed for this ion. Glc: glucose; cps, counts per second.

FIGS. 20A-20D are plots showing expression analysis of stilbene biosynthesis related genes in wild sugarcane. FIGS. 20A-B are a pair of plots showing quantitative RT-PCR gene expression analysis of SsSTS (FIG. 20A) and SsCOMT (FIG. 20B). Expression levels are expressed relative to Saccharum spontaneum Glyceraldehyde 3-Phosphate Dehydrogenase (SsGADPH)⁶⁸. Values refer to means±SD (n=5). FIGS. 20C-20D are semi-quantitative RT-PCR gene expression analysis of SsSTS (FIG. 20C) and SsCOMT (FIG. 20D) relative to SsGADPH. Thirty cycles were used for amplification. Two biological duplicates from each time point were analyzed.

FIG. 21 represents multiple sequence alignment of SbSTS1 and SsSTS. The amino acid sequences of SbSTS1 and SsSTS were aligned using Clustal Omega⁸⁸. Fully conserved amino acid residues are indicated by asterisks; those with strongly similar properties are indicated with colons; those with weakly similar properties are indicated with periods. The sequences are SEQ ID NO:23 (SbSTS1) and SEQ ID NO:33 (SsSTS).

FIGS. 22A-22E demonstrate catalytic activities of SsSTS. FIG. 22A is a schematic showing proposed STS reaction with feruloyl-CoA and malonyl-CoA as substrates. FIGS. 22B-22D are plots showing HPLC-QTOF-HRMS detection of isorhapontigenin (FIG. 22B), 3′-methoxybisnoryangonin (FIG. 22C), and feruloyltriacetic acid lactone (FIG. 22D) in SsSTS catalyzed reaction using feruloyl-CoA and malonyl-CoA as substrates. The detection of these derailment products is consistent with a previous report showing that feruloyl-CoA is a poor substrate for STSs, at least under in vitro conditions⁵. FIG. 22E is a plot showing HPLC-QTOF-HRMS detection of piceatannol in SsSTS catalyzed reaction using caffeoyl-CoA and malonyl-CoA as substrates. cps, counts per second; NC, negative control (reaction without enzyme).

FIG. 23 represents multiple sequence alignment of SbCOMT, SbSOMT, and SsCOMT. The amino acid sequences of SbCOMT, SbSOMT, and SsCOMT were aligned using Clustal Omega⁸⁸. Fully conserved amino acid residues are indicated by asterisks; those with strongly similar properties are indicated with colons; those with weakly similar properties are indicated with periods. Residues of SbSOMT and SsCOMT) corresponding to SbCOMT Asn128 and Asn323 were underlined and bolded. Sequences SEQ ID NO:1 (SbSOMT), SEQ ID NO:2 (SbCOMT), and SEQ ID NO:32 (SsCOMT).

FIGS. 24A-24I show catalytic activities of SsCOMT. FIG. 24A represents in vitro O-methylation of piceatannol by SsCOMT. FIG. 24B is a plot showing high-performance liquid chromatography-tandem MS (HPLC-MS/MS) detection of isorhapontigenin in SsCOMT catalyzed reaction using resveratrol as a substrate. FIG. 24C represents in vitro O-methylation of caffeic acid by SsCOMT. FIG. 24D is a plot showing high-performance liquid chromatography-tandem MS (HPLC-MS/MS) detection of ferulic acid in SsCOMT catalyzed reaction using caffeic acid as a substrate. FIG. 24E represents in vitro O-methylation of 5-hydroxyferulic acid by SsCOMT. FIG. 24F is a plot showing high-performance liquid chromatography-tandem MS (HPLC-MS/MS) detection of sinapic acid in SsCOMT catalyzed reaction using 5-hydroxyferulic acid as a substrate. FIG. 24G represents in vitro O-methylation of resveratrol by SsCOMT. FIGS. 24H-24I are a pair of plots showing high-performance liquid chromatography-tandem MS (HPLC-MS/MS) detection of pinostilbene (FIG. 24H), and pterostilbene (FIG. 24I) in SsCOMT catalyzed reaction using resveratrol as a substrate. cps, counts per second; NC, negative control (reaction without enzyme).

FIG. 25 is a structural model that explains the basis of polarity pairing via the representative SbSOMT-resveratrol interaction. The structure shows that at the proximity of catalytic dyad (Hiscat and Aspcat), the non-polar Phe337 of SbSOMT is closely positioned to non-polar C4 (the vicinal group of reactive site of resveratrol) and demonstrate the coordination of substrate orientation via polarity pairing. In SbCOMT context (bottom right box), the polar Asn323 is positioned at the equivalent location of Phe337 of SbSOMT. Hence, SbCOMT coordinates via the opposite polar-to-polar polarity pairing.

FIGS. 26A-26X are structural models showing polarity pairing as a common regioselective mechanism across OMT-ligand complexes deposited to PDB. Twenty-four unique OMT-ligand complexes were retrieved from PDB (by 10 Aug. 2022), and their respective amino acid residue equivalent to SbCOMT^{Asn323}/SbSOMT^{Phe337}, catalytic dyad, and bound ligand were shown. Homolog search was used to retrieve (≥30% identity to SbCOMT or SbSOMT) and screened for complexes that are bound to ligand at substrate binding pocket. Further details are listed in Table 10. Appropriate polarity pairing between this residue's side chain and the functional group vicinal to the reactive site (methyl-accepting —OH group) was observed in all complexes, except for the three structures excluded from the analysis employed herein, either due to the use of non-reactive ligand (3I5U and 5ICF) or non-productive OMT-ligand conformation (5I2H). The carbon vicinal to the reactive site is circled in black.

FIGS. 27A-27M demonstrate in vitro toxicity of stilbenes towards Colletotrichum sublineola. FIGS. 27A-27I are images showing mycelial growth of C. sublineola on potato dextrose agar after a 72-hour incubation with 4% (v/v) DMSO (FIG. 27A), 50 μM resveratrol (FIG. 27B), 50 μM pinostilbene (FIG. 27C), 5 μM pterostilbene (FIG. 27D), 10 μM pterostilbene (FIG. 27E), 20 μM pterostilbene (FIG. 27F), 30 μM pterostilbene (FIG. 27G), 40 μM pterostilbene (FIG. 27H) and 50 μM pterostilbene (FIG. 27I). Scale bars denote 5 mm. FIGS. 27J-27L are images that show spore germination of C. sublineola on potato dextrose agar after a 12-hour incubation in darkness with 4% (v/v) DMSO (FIG. 27J), 40 μM pterostilbene (FIG. 27K) and 50 μM pterostilbene (FIG. 27L). Scale bars denote 500 μm. FIG. 27M is a plot showing diameter of C. sublineola colonies after a 72-hour incubation with different concentrations of pterostilbene. IC50 was estimated by GraphPad Prism 6. Values refer to means±SD (n=9). IC50, half-maximal inhibitory concentration.

FIG. 28 is an image showing arrangement of SbSTS1 (Sb07g004700; Sobic.007G058900), SbOMT4 (Sb07g004690; Sobic.007G058800), and SbSOMT (Sb07g004710; Sobic.007G059100) in Sorghum genome. Gene arrangement in Chr07:6094677 . . . 6186377 of Sorghum genome was obtained from Phytozome v13⁶⁵.

FIGS. 29A-29L are plots showing MSMS spectrum of stilbenes detected in plant samples and authentic standards. FIGS. 29A-29B are representative MSMS spectrum of resveratrol detected by HPLC-QTOF-HRMS in (FIG. 29A) C. sublineola-infected Sorghum mesocotyls or mechanically-wounded wild sugarcane, and (FIG. 29B) authentic resveratrol standard. FIGS. 29C-29D are representative MSMS spectrum of piceid detected by HPLC-QTOF-HRMS in (FIG. 29C) C. sublineola-infected Sorghum mesocotyls, and (FIG. 29D) authentic piceid standard. FIGS. 29E-29F are representative MSMS spectrum of pinostilbene detected by HPLC-QTOF-HRMS in (FIG. 29E) C. sublineola-infected Sorghum mesocotyls, and (FIG. 29F) authentic pinostilbene standard. FIGS. 29G-29H are representative MSMS spectrum of pterostilbene detected by HPLC-QTOF-HRMS in (FIG. 29G) C. sublineola-infected Sorghum mesocotyls, and (FIG. 29H) authentic pterostilbene standard. FIGS. 29I-29J are representative MSMS spectrum of piceatannol detected by HPLC-QTOF-HRMS in (FIG. 29I) mechanically-wounded wild sugarcane, and (FIG. 29J) authentic piceatannol standard. FIGS. 29K-29L are representative MSMS spectrum of isorhapontigenin detected by HPLC-QTOF-HRMS in (FIG. 29K) mechanically-wounded wild sugarcane, and (FIG. 29L) authentic isorhapontigenin standard. cps, counts per second; m/z, mass-to-charge ratio.

### DETAILED DESCRIPTION OF THE INVENTION

The disclosed method and compositions can be understood more readily by reference to the following detailed description of particular embodiments and the Example included therein and to the Figures and their previous and following description.

O-methyltransferases (OMTs) are ubiquitous enzymes in plants and are involved in a type of chemical modification, known as O-methylation, of phenolic natural products. Methylation of these compounds generally enhances their hydrophobicity, which confers higher bioavailability after intake and thus significantly increases the potency of their health-promoting properties. A biochemical mechanism termed compatible polarity pairing has been discovered that determines the catalytic regioselectivity in O-methylation of phenolic hydroxyl (—OH) groups in Type 1 OMTs. The discovery is based on the identification of a key amino acid position within Type 1 OMTs (corresponding or equivalent to Phe337 of SbSOMT), which, if the said amino acid is highly polar (or hydrophilic, e.g. asparagine) in nature, preferentially interacts with the polar functional group (e.g. —OH and —OMe) of an incoming aromatic ring, thus positioning the —OH group(s) adjacent to the polar functional group for O-methylation. In contrast, if the said amino acid is hydrophobic in nature (e.g. phenylalanine, valine, threonine), it preferentially interacts with an aromatic carbon in the phenolic ring of an incoming substrate, thus positioning the —OH group(s) adjacent to the aforementioned aromatic carbon for O-methylation. Thus, a structure-function relationship for the nature of the amino acid that corresponds to Phe337 of SbSOMT allows prediction and alteration of the regioselectivity of substrated for modified OMTs.

In the case of SbSOMT, Phe337 interacts with C4 of the stilbene A-ring and the C3-OH group is positioned for O-methylation. On the other hand, the equivalent residue in SbCOMT is Asn323 which interacts with 4′-OH group of the stilbene B-ring, hence t3′-OH group is positioned for O-methylation. Consistent with this, we discovered a panel of 24 Type 1 OMT-ligand complexes that supports this same compatible polarity paring between the amino acid residue equivalent to SbSOMT Phe337 and SbCOMT Asn323 and their substrates. These Type 1 OMT enzymes all utilize the same catalytic mechanisms to catalyze O-methylation of phenolic substrates. Hence, our discovery allows the predictable engineering of Type 1 OMTs with an appropriate residue at the corresponding position for regiospecific O-methylation of phenolic substrates.

Previous attempts at biosynthesis of O-methylated phenolic compounds relied on the trial-and-error approach. They relied solely on the natural capability of Type 1 OMTs to catalyze O-methylation of phenolic substrates in vivo. Hence, both the OMTs involved and the resultant end products were unpredictable and had to be experimentally tested which is time- and resource-consuming. Optimization of Type 1 OMT-regioselectivity to tailor make the end products was very challenging and, again, relied on trial-and-error. Thus, the mechanism underlying the catalytic regioselectivity of O-methyltransferases at the heart of our discovery had long been sought after.

With our discovery, biosynthesizing O-methylated phenolic natural products in a biosystem now becomes highly precise and predictable. Our discovery also allows the synthesis of novel O-methylated phenolic compounds.

Our discovery allows (1) straightforward prediction of Type 1 OMT(s) responsible for catalyzing certain —OH groups of phenolic substrates, (2) rational choice of Type 1 OMT(s) for targeted compound biosynthesis and synthetic biology, and (3) optimization of Type 1 OMT catalytic activity and regioselectivity by protein engineering.

The underlying biochemical mechanism determining the regioselectivity of Type 1 OMTs was largely unknown until our discovery here. Accordingly, the incorporation of a hydrophobic amino acid at position equivalent to SbSOMT Phe337 in any OMT will greatly favor the O-methylation of —OH group(s) adjacent to the interacting aromatic carbon of a phenolic ring in a substrate. In contrast, the incorporation of a hydrophilic amino acid at position equivalent to SbSOMT Phe337 in any OMT will greatly favor the O-methylation of —OH group(s) adjacent to an interacting polar functional group of a phenolic ring in a substrate.

Disclosed are modified Type 1 O-methyltransferases (OMTs) and methods of using such modified OMTs. In some forms of the modified OMTs, the amino acid corresponding to Phe337 of Sorghum bicolor stilbene OMT (SbSOMT) is substituted with a polar amino acid. In some forms of the modified OMTs, the amino acid corresponding to Asn323 of Sorghum bicolor caffeic acid OMT (SbCOMT) is substituted with a hydrophobic amino acid. In some forms of the modified OMTs, the amino acid corresponding to Ile144 of SbSOMT is substituted with a polar amino acid. In some forms of the modified OMTs, the amino acid corresponding to Asn128 of SbCOMT is substituted with a hydrophobic amino acid.

In some forms, the substituting polar amino acid is independently Asn, Gln, Thr, or Ser. In some forms, the substituting polar amino acid is Asn. In some forms, the substituting hydrophobic amino acid is independently Phe, Leu, Trp, Tyr, Ile, Met, Val, or Ala. In some forms, the substituting hydrophobic amino acid is Phe.

In some forms, the modified OMT is a modified form of Arabidopsis thaliana AtOMT1, Carthamus tinctorius CtCAldOMT, Catharanthus roseus CrCOMT, Hordeum vulgare HvCOMT, Medicago sativa MsCOMT, Medicago sativa MsIOMT, Oryza sativa OsCAldOMT1, Oryza sativa OsNOMT, Panicum virgatum PvCOMT, Pinus sylvestris PsPMT2, Pinus taeda PtAEOMT, Populus tremuloides PtCOMT, Rosa hybrid RhOOMT, Saccharum spontaneum SsSTS, Saccharum spontaneum SsCOMT, Solanum lycopersicum SICOMT, Sorghum bicolor SbCOMT, Sorghum bicolor SbNOMT, Sorghum bicolor SbOMT1, Sorghum bicolor SbOMT3, Sorghum bicolor SbOMT4, Sorghum bicolor SbSOMT, Sorghum bicolor SbSTS1, Sorghum halepense ShOMT1, Sorghum halepense ShSOMT, Triticum aestivum TaCOMT1, Triticum aestivum TaCOMT2, Vitis vinifera VvCOMT, Vitis vinifera VvROMT, Zea mays ZmCOMT, Zea mays ZmNOMT, Medicago truncatula MtIOMT, Medicago truncatula MtIOMT3, Streptomyces carzinostaticus ScNOMT, Loium perenne LpCOMT, Clarkia breweri CbIEOMT, Planctopirus limnophila PlOMT, Thalictrum flavum TfNOMT, Kitagawia praeruptora KpBOMT, Papaver somniferum PsSOMT, or Fragaria ananassa FaOMT.

In some forms, the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93. In some forms, the modified OMT has at least 70% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT has at least 75% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT has at least 80% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT has at least 85% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT has at least 90% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT has at least 95% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

In some forms, the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Phe337 of Sorghum bicolor stilbene OMT (SbSOMT) substituted with a polar amino acid.

In some forms, the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Asn323 of Sorghum bicolor caffeic acid OMT (SbCOMT) substituted with a hydrophobic amino acid.

In some forms, the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Ile144 of SbSOMT substituted with a polar amino acid.

In some forms, the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Asn128 of SbCOMT substituted with a hydrophobic amino acid.

In some forms, the modified OMT is further modified to improve or alter the production of an O-methylated stilbenoid or hydroxycinnamic acid by the modified OMT.

Also disclosed are solutions including a modified OMT as disclosed. In some forms, the solution is composed to allow production of one or more O-methylated forms of stilbenoid or hydroxycinnamic acid from a substrate for the modified OMT under suitable conditions. Also disclosed are plants that express a modified OMT as disclosed.

Also disclosed are methods of producing an O-methylated stilbenoid or hydroxycinnamic acid. In some forms, the method involves bringing into contact, in a solution, a modified OMT as disclosed, a substrate for the modified OMT, and S-adenosylmethionine. In general, the O-methylated stilbenoid or hydroxycinnamic acid is produced by incubating the solution under suitable conditions.

In some forms, the method involves growing that express a modified OMT as disclosed and extracting the O-methylated stilbenoid or hydroxycinnamic acid from the grown plant.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

The disclosed compositions and methods can be further understood through the following numbered paragraphs.

1. A modified Type 1 O-methyltransferase (OMT), wherein the amino acid corresponding to Phe337 of Sorghum bicolor stilbene OMT (SbSOMT) is substituted with a polar amino acid or wherein the amino acid corresponding to Asn323 of Sorghum bicolor caffeic acid OMT (SbCOMT) is substituted with a hydrophobic amino acid.

2. The modified OMT of paragraph 1, wherein the amino acid corresponding to Ile144 of SbSOMT is substituted with a polar amino acid or wherein the amino acid corresponding to Asn128 of SbCOMT is substituted with a hydrophobic amino acid.

3. The modified OMT of paragraph 1 or 2, wherein the substituting polar amino acid is independently Asn, Gln, Thr, or Ser.

4. The modified OMT of any one of paragraphs 1-3, wherein the substituting polar amino acid is Asn.

5. The modified OMT of any one of paragraphs 1-4, wherein the substituting hydrophobic amino acid is independently Phe, Leu, Trp, Tyr, Ile, Met, Val, or Ala.

6. The modified OMT of any one of paragraphs 1-5, wherein the substituting hydrophobic amino acid is Phe.

7. The modified OMT of any one of paragraphs 1-6, wherein the modified OMT is a modified form of Arabidopsis thaliana AtOMT1, Carthamus tinctorius CtCAldOMT, Catharanthus roseus CrCOMT, Hordeum vulgare HvCOMT, Medicago sativa MsCOMT, Medicago sativa MsIOMT, Oryza sativa OsCAldOMT1, Oryza sativa OsNOMT, Panicum virgatum PvCOMT, Pinus sylvestris PsPMT2, Pinus taeda PtAEOMT, Populus tremuloides PtCOMT, Rosa hybrid RhOOMT, Saccharum spontaneum SsSTS, Saccharum spontaneum SsCOMT, Solanum lycopersicum SICOMT, Sorghum bicolor SbCOMT, Sorghum bicolor SbNOMT, Sorghum bicolor SbOMT1, Sorghum bicolor SbOMT3, Sorghum bicolor SbOMT4, Sorghum bicolor SbSOMT, Sorghum bicolor SbSTS1, Sorghum halepense ShOMT1, Sorghum halepense ShSOMT, Triticum aestivum TaCOMT1, Triticum aestivum TaCOMT2, Vitis vinifera VvCOMT, Vitis vinifera VvROMT, Zea mays ZmCOMT, Zea mays ZmNOMT, Medicago truncatula MtIOMT, Medicago truncatula MtIOMT3, Streptomyces carzinostaticus ScNOMT, Loium perenne LpCOMT, Clarkia breweri CbIEOMT, Planctopirus limnophila PlOMT, Thalictrum flavum TfNOMT, Kitagawia praeruptora KpBOMT, Papaver somniferum PsSOMT, or Fragaria ananassa FaOMT.

8. The modified OMT of any one of paragraphs 1-7, wherein the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

9. The modified OMT of any one of paragraphs 1-8, wherein the modified OMT has at least 70% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

10. The modified OMT of any one of paragraphs 1-9, wherein the modified OMT has at least 75% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

11. The modified OMT of any one of paragraphs 1-10, wherein the modified OMT has at least 80% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

12. The modified OMT of any one of paragraphs 1-11, wherein the modified OMT has at least 85% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

13. The modified OMT of any one of paragraphs 1-12, wherein the modified OMT has at least 90% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

14. The modified OMT of any one of paragraphs 1-13, wherein the modified OMT has at least 95% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

15. The modified OMT of any one of paragraphs 1-14, wherein the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Phe337 of Sorghum bicolor stilbene OMT (SbSOMT) substituted with a polar amino acid.

16. The modified OMT of any one of paragraphs 1-15, wherein the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Asn323 of Sorghum bicolor caffeic acid OMT (SbCOMT) substituted with a hydrophobic amino acid.

17. The modified OMT of any one of paragraphs 1-16, wherein the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Ile144 of SbSOMT substituted with a polar amino acid.

18. The modified OMT of any one of paragraphs 1-17, wherein the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Asn128 of SbCOMT substituted with a hydrophobic amino acid.

19. The modified OMT of any one of paragraphs 1-18, wherein the modified OMT is further modified to improve or alter the production of an O-methylated stilbenoid or hydroxycinnamic acid by the modified OMT.

20. A solution comprising the modified OMT of any one of paragraphs 1-19, wherein the solution is composed to allow production of one or more O-methylated forms of stilbenoid or hydroxycinnamic acid from a substrate for the modified OMT under suitable conditions.

21. A method of producing an O-methylated stilbenoid or hydroxycinnamic acid, the method comprising bringing into contact, in a solution, the modified OMT of any one of paragraphs 1-19, a substrate for the modified OMT and S-adenosylmethionine; and incubating under suitable conditions to produce the O-methylated stilbenoid or hydroxycinnamic acid.

22. A plant that expresses the modified OMT of any one of paragraphs 1-19.

23. A method of producing an O-methylated stilbenoid or hydroxycinnamic acid, the method comprising growing the plant of paragraph 22 and extracting the O-methylated stilbenoid or hydroxycinnamic acid from the grown plant.

### EXAMPLES

O-Methylated stilbenes are prominent nutraceuticals but rarely produced by crops. Here, the inherent ability of two Saccharinae grasses to produce regioselectively O-methylated stilbenes is reported. A stilbene O-methyltransferase, SbSOMT, is first shown to be indispensable for pathogeninducible pterostilbene (3,5-bis-O-methylated) biosynthesis in Sorghum (Sorghum bicolor). Phylogenetic analysis indicates the recruitment of genus-specific SOMTs from canonical caffeic acid O-methyltransferases (COMTs) after the divergence of Sorghum spp. from Saccharum spp. In recombinant enzyme assays, SbSOMT and COMTs regioselectively catalyze O-methylation of stilbene A-ring and B-ring respectively. Subsequently, SOMT-stilbene crystal structures are presented. Whilst SbSOMT shows global structural resemblance to SbCOMT, molecular characterizations illustrate two hydrophobic residues (Ile144/Phe337) crucial for substrate binding orientation leading to 3,5-bis-Omethylations in the A-ring. In contrast, the equivalent residues (Asn128/Asn323) in SbCOMT facilitate an opposite orientation that favors 3′-O-methylation in the B-ring. Consistently, a highly-conserved COMT is likely involved in isorhapontigenin (3′-O-methylated) formation in wounded wild sugarcane (Saccharum spontaneum). Altogether, our work reveals the potential of Saccharinae grasses as a source of O-methylated stilbenes, and rationalize the regioselectivity of SOMT activities for bioengineering of O-methylated stilbenes.

Within the family Poaceae, the Sorghum and Saccharum genera evolved from a common ancestral grass and belong to the tribe Andropogonae, subtribe Saccharinae. Species from both genera accumulate hydroxylated stilbenes (e.g. resveratrol, piceatannol) and their O-glycosides^{5, 7, 36}, but O-methylated stilbenes have not been reported in these species so far. Here, we report the identification of O-methylated stilbenes, pinostilbene (3-O-methylated) and pterostilbene (3,5-bis-O-methylated) in Sorghum, and isorhapontigenin (3′-O-methylated) in wild sugarcane (S. spontaneum). A stilbene OMT (SbSOMT) catalyzes sequential 3,5-bis-O-methylation of resveratrol to pterostilbene whereas a canonical COMT (SsCOMT) likely catalyzes 3′-O-methylation of piceatannol into isorhapontigenin in wild sugarcane. We generated Sorghum SbSOMT CRISPR/Cas9 mutants which were deficient in pinostilbene and pterostilbene, hence establishing the indispensable role of SbSOMT in resveratrol 3,5-bis-O-methylation. As indicated by phylogenetic analysis, divergence of Sorghum genus from other genera in the Saccharinae subtribe likely predated the recruitment of SbSOMT from a canonical COMT. By solving the high-resolution crystal structures of SbSOMT complexed with stilbenes, molecular features that differentiate the substrate binding mode and catalytic regioselectivity between SbSOMT and canonical COMTs were unveiled.

### Methods

### Plant Materials and Fungal Treatment Conditions

Two Sorghum (Sorghum bicolor) genotypes, BTx623 and SC748-5, were analyzed in this study. Sorghum seeds were germinated in aerated double-deionized water at 30° C. for 24 h. Wild sugarcane (Saccharum spontaneum) sets were obtained from Taiwan⁶⁶, and planted in soil until flowering. C. sublineola isolate TX430BB was propagated on 5% (w/v) oatmeal agar for 14 days prior to fungal infection experiments. Etiolated Sorghum seedlings were prepared as described previously⁶⁷, and were either sprayed with a 0.2% (w/v) bovine gelatin solution (control) or a conidial suspension (1.0×106 spores ml-1) of C. sublineola isolate TX430BB in the same gelatin solution (treatment). Both control and treatment groups were incubated under constant light at room temperature and 100% humidity.

### Plant Metabolite Extraction

One hundred milligrams of Sorghum mesocotyls from fungal infection experiments or mechanically wounded wild sugarcane stalks were harvested for metabolite analysis. All plant tissues were frozen by liquid nitrogen and ground into fine powder using TissueLyser II (QIAGEN, Germany). Two hundred microliters of 80% (v/v) methanol (with 10 μM Apigenin-d₅ as internal standard) were added to the samples. Metabolites were extracted by ultra-sonicating the samples on an ice-water bath for 30 min. Samples were passed through a 0.22 μm PTFE membrane filter (Phenomenex, USA) prior to HPLC-QTOF-HRMS analysis described below.

### Hplc-QTOF-HRMS and HPLC-MRM Analyses

To screen for OMT activities, purified stilbene products of enzyme assays were separated by a Synergi C18 column (Synergi 4μ Fusion RP 80 Å, 50×2 mm, Phenomenex, USA) under a flow rate of 0.5 ml min-1 over a 6 min linear gradient of 10%-90% B (described below). Product detection was achieved with a quadruple-time-of-flight-high resolution mass spectrometer (QTOF-HRMS) X500R system (AB Sciex, China) operating under the information-dependent acquisition (IDA) mode. Meanwhile, purified phenylpropanoid products of enzyme assays were separated on the same column connected to a HP1100 series HPLC system (Agilent Technologies, USA) linked to an AP3200-QTRAP mass spectrometer (AB Sciex, China) operating under the multiple-reaction-monitoring (MRM) mode. To quantify these products, assay products were separated on a Kinetex C18 column (Kinetex 2.6 μm C18 100 Å, 100×2.1 mm, Phenomenex, USA) connected to a HP1100 series HPLC system (Agilent Technologies, USA) linked to an AP3200-QTRAP mass spectrometer (AB Sciex, China) operating under the multiple-reaction-monitoring (MRM) mode. A linear gradient of 10%-90% B under a flow rate of 0.3 ml min-1 over 6 min was used to further enhance sensitivity. Total metabolites from all plant tissues were separated by the same Kinetex C18 column connected to the X500R QTOF-HRMS system. A linear gradient under a flow rate of 0.3 ml min-1 over a 20 min linear gradient of 10%-90% B was used.

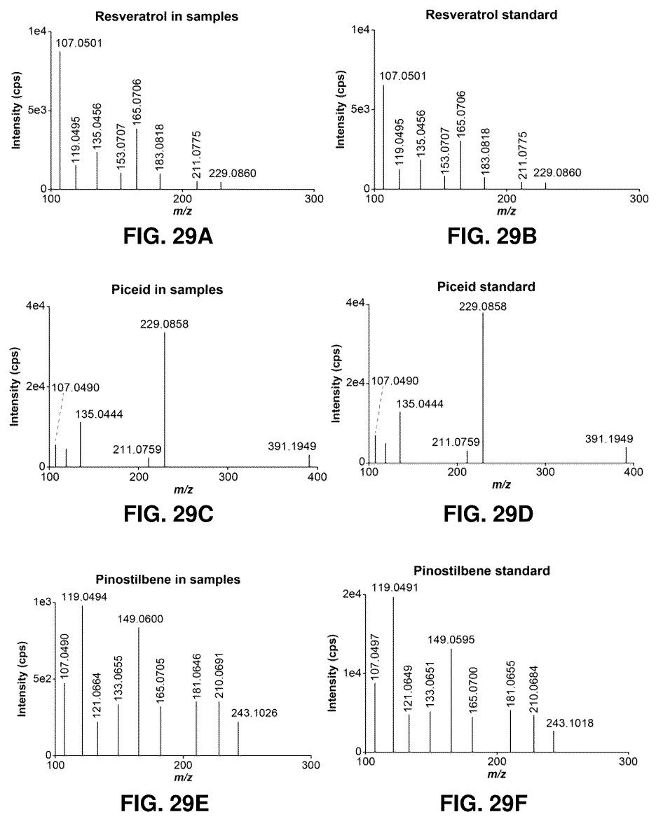

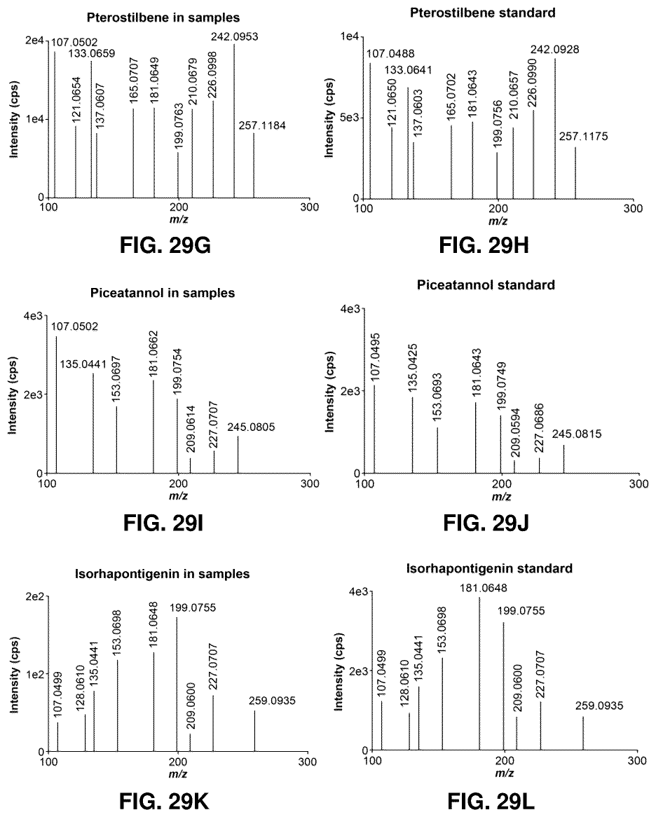

In all HPLC-MS analyses, the mobile phase consisted of 0.5% (v/v) formic acid/water (A) and 0.5% (v/v) formic acid/methanol (B). Stilbenes were detected using the positive ionization mode while phenolic acids were detected with the negative ionization mode. Compounds were quantified by integration of peak area using the quantification mode of SCIEX OS (for X500R) or Analyst software version 1.5.6 (for AP3200-QTRAP). Identification of compounds was achieved by comparing the retention time and MS/MS spectra (FIGS. 29A-29L) with authentic standards.

### Gene Expression Analyses

Total RNA was extracted from Sorghum mesocotyls collected at a 24-hour interval for up to 96 h post fungal-treatment and wounded wild sugarcane stalks collected at different time points using the TRIzol method (Invitrogen, USA). Reverse transcription and qRT-PCR were performed using PrimeScript RT reagent kit with gDNA eraser (TaKaRa, Japan) and TB Green premix Ex Taq II kit (TaKaRa, Japan), respectively. Semi qRT-PCR was performed with GoTaq DNA polymerase (Promega, USA). Gene-specific primers used for qRT-PCR experiments were listed in Table 11. The housekeeping gene Sorghum bicolor Eukaryotic Initiation Factor 4A-1 (SbEIF4α⁶⁸; XM_002451491) and Saccharum spontaneum Glyceraldehyde 3-Phosphate Dehydrogenase (SsGADPH; Sspon.08G0001560-1A)⁶⁹ were used as internal controls for Sorghum and wild sugarcane, respectively.

### Cloning of SbSTS1, SbOMT4, SbSOMT, SbCOMT, SsSTS, and SsCOMT

The coding sequences (CDS) of SbSTS1, SbOMT4, SbSOMT and SbCOMT were amplified from cDNA prepared from fungal-infected mesocotyls of Sorghum genotype BTx623 using gene-specific primers (Table 11). The CDS of SsSTS and SsCOMT were cloned from cDNA prepared from wounded wild sugarcane stalk using gene-specific primers (Table 11). To generate recombinant proteins, the CDS of SsSTS (full length), SsCOMT (full length), SbCOMT (encoding residues 2-362), and SbSOMT (encoding residues 2-377) were cloned into pET-N-His-TEV (Beyotime) via HiFi DNA Assembly (New England BioLabs, USA) or Gibson Assembly (New England BioLabs, USA). Full length CDS of SbOMT4 was inserted between BamHI and HindIII restriction sites of pET23a(+) vector (Novagen, Germany). The full length CDSs of SbSTS1 and SbSOMT were individually subcloned into the binary vector pEAQ-HT by the Gibson Assembly method (New England BioLabs, USA) using gene-specific primers (Table 11).

### Recombinant Protein Production and Site-Directed Mutagenesis

Desired mutations of SbSOMT were introduced into its construct using specific primers (Table 11). Expression constructs were transformed into Rosetta™ (DE3) competent Escherichia coli cells (Novagen, Germany). Protein expression was induced with 0.1 mM Isopropyl-β-thiogalactopyranoside (IPTG) overnight at 16° C. E. coli cells were harvested, resuspended in lysis buffer (20 mM Tris buffer pH 7.9, 300 mM NaCl, 2.5 mM β-mercaptoethanol), and lysed by sonication. Crude proteins were loaded onto a HisTrap HP column (Cytiva, USA) connected to a AKTA pure chromatography system (Cytiva, USA). Column was washed with 15 column volumes of lysis buffer with 50 mM imidazole. Proteins were eluted over a gradient of 50-500 mM imidazole. Eluate fractions with the target OMT were pooled, digested with TEV protease, and simultaneously dialyzed against a low salt buffer (20 mM Tris pH 7.9, 50 mM NaCl, 2.5 mM β-mercaptoethanol) overnight at 4° C. The dialyzed sample was filtered and flowed through HisTrap to remove His-tagged components. The flowthrough was then loaded to HiTrap Q FF columns (Cytiva, USA) over a gradient of 50-1000 mM NaCl. Polishing was achieved via size-exclusion chromatography using the storage buffer (100 mM HEPES pH 7.9, 100 mM NaCl) as mobile phase. SsSTS was purified under the same conditions except that the pH was adjusted to 7.0 for all buffers. Proteins were concentrated by ultrafiltration and flash frozen for storage at −80° C.

### Enzyme Assays and Enzyme Kinetics

For initial screening of OMT activities, purified OMT enzymes (10 μg) were incubated in 100 mM HEPES buffer (pH 7.9), 200 μM S-adenosyl-L-methionine (SAM) and 100 μM of phenylpropanoid or stilbene substrates (final volume 200 μl) at 30° C. for 2 h. Enzyme kinetics were determined by incubating 1 μg protein with 100 mM HEPES buffer (pH 7.9), 300 μM SAM, and stilbene concentrations ranging from 5 to 200 μM at 30° C. for 5 min. Similar conditions were used to determine enzyme kinetics of SbSOMT towards SAM, except that SAM concentrations ranging from 5 to 500 μM (while stilbene substrates were kept at 1 mM) were included to accommodate its unexpectedly high Kd and Km values. For the SsSTS enzyme assays, 10 μg of purified SsSTS enzymes were incubated in 100 mM HEPES buffer (pH 7.0), 100 μM of caffeoyl-CoA or feruloyl-CoA, and 300 μM of malonyl-CoA at 30° C. for 1 h. All enzyme assay reactions were quenched by snap-freezing the reaction tubes in liquid nitrogen. Reaction products were extracted twice with ethyl acetate. The organic layers were then pooled, vacuum-dried, and resuspended in 50 μl 80% (v/v) methanol with 10 μM tricin as internal standard for HPLC-MRM quantification. All reactions were done in triplicates. Enzyme kinetics were calculated using the non-linear regression fitting by GraphPad Prism 6 software (GraphPad, USA).

### Isothermal Titration Calorimetry (ITC)

Isothermal titration calorimetry (ITC) assays were conducted with MicroCal iTC200 (Malvern Panalytical, UK). Concentrated stocks of resveratrol, pinostilbene, pterostilbene, and piceatannol were prepared in 50% (v/v) PEG 400. Both cell sample and titrant were equilibrated to ITC buffer consisting of 100 mM HEPES pH 7.9 with 2.5% (v/v) PEG 400. For ITC assays involving pterostilbene, PEG 400 concentration was increased to 5% (v/v). Concentrations of protein (in cell) and ligand (in syringe) used in each run are listed in Table 11. Each run incorporated an initial delay of 60 s prior to the first injection (0.5 μl) and spaced 150 s between the subsequent 19 injections (2.0 μl) at 25° C. During the runs, the cell was continuously stirred at 750 rpm by flat paddle. Results were analysed based on the 2nd to 20th injections using the Origin (MicroCal Software, USA) and PEAQ-ITC software (Malvern Panalytical, UK).

Agroinfiltration of N. benthamiana Leaves

pEAQ constructs harboring either SbSTS1 or SbSOMT were transformed into Agrobacterium tumefaciens strain GV3101. To co-express SbSTS1 and SbSOMT in N. benthamiana leaves, two Agrobacterium cultures, each harboring one of the overexpression plasmids (individual OD600 at 0.8), were mixed in equal ratio and co-infiltrated to N. benthamiana leaves. Leaves were harvested five days after infiltration and were subjected to metabolite extraction and HPLC-QTOF-HRMS analysis as described above.

### Constructs of CRISPR-SbSOMT and Sorghum Transformation

The CRISPR-SbSOMT construct was chemically synthesized (Gene Universal, USA). In brief, two gRNAs, including gRNA1: GTTGAACACGGTGTTCCACG and gRNA2: GCACCGGACTACGCTGTGCG, were designed using CHOPCHOP v3⁷⁰, individually introduced to the downstream of SbU6 promoters, SbU62.3 and SbU63.1, respectively, and were further cloned into the plasmid that harbors a selective marker (NPTII) to generate CRISPR-SbSOMT construct as described previously⁵⁰. The CRISPR/Cas9 plasmid, pBUN411, was modified for Sorghum particle bombardment⁷¹. Sorghum tissue culture, transformation, and CRISPR-Cas9-mediated genome editing were performed as described previously⁴⁹. Briefly, Sorghum inbred line Tx430 were grown in a temperature-controlled (18-28° C.) glasshouse to provide the initial explant for transformation. Immature seeds were harvested 11-15 days post anthesis. Immature embryos were isolated and placed on callus induction medium for generating embryogenic calli. The CRISPR-SbSOMT construct and pBUN411 plasmid were co-transformed into embryogenic calli by particle bombardment as described previously⁷². After transformation, potential transgenic plantlets were grown in a temperature-controlled (18-28° C.) glasshouse. Genome-edited plants (T0 and T1) were identified by PCR and direct sequencing using primers listed in Table 11.

### X-Ray Crystallography

SbSOMT was prepared at 3.5 mg ml-1 and pre-equilibrated with 0.5 mM of stilbene ligand in a final buffer of 20 mM HEPES pH 7.9, 150 mM NaCl, 4.5% dimethyl sulfoxide (DMSO) and 1 mM TCEP. Co-crystallization of SbSOMT and resveratrol were initially screened against commercial crystallization screens (Hampton Research, USA and QIAGEN, USA) with a sitting drop vapor diffusion approach incubated at 18° C. Two crystallizing conditions were further optimized. Crystals of SbSOMT-resveratrol-β-NAD ternary complex were obtained after two days of growing from the setup of 1 μl of 5 mg ml-1 SbSOMT, 0.15 μl of 50 mM β-NAD and 0.5 μl of reservoir solution (0.1 M sodium acetate pH 4.6, 0.2 M sodium acetate, 0.2 M NH4Cl, 2.5% (w/v) polyethylene glycol 4000). SbSOMT co-crystalized with pinostilbene, piceatannol, or pterostilbene were obtained under the same condition. Crystal of SbSOMT-resveratrol binary complex was initially obtained after 2-3 weeks of growing from the setup of 1 μl of 5 mg ml-1 SbSOMT and 0.5 μl of reservoir solution (0.1 M MES pH 6.5, 0.2 M ammonium sulphate, 30% (w/v) polyethylene glycol monomethyl ether 5000). Subsequent crystallization was accelerated by seeding and crystals were harvested after 1 week. Crystals collected for X-ray diffraction was cryoprotected by 20% (v/v) glycerol topped onto the reservoir solution.

X-ray diffraction data were collected from BL19U1 beamline (wavelength at 0.979 Å) at the Shanghai Synchrotron Radiation Facility and processed by either XDS or HKL3000 package⁷³⁻⁷⁴. Data were analysed using pipelines provided in CCP4 suite⁷⁵. Data reduction was performed using AIMLESS⁷⁶. The initial structure of SbSOMT-resveratrol-β-NAD ternary complex was solved by molecular replacement; first through PHASER using a chimeric search model generated by MrBUMP, followed by model rebuilding from phase solution through BUCCANEER⁷⁷⁻⁸¹. Following structures were solved by molecular replacement with PHASER using protomer of SbSOMT-resveratrol-β-NAD ternary complex as a search model. The built models were iteratively refined through automated REFMAC5 refinement and manual refinement in COOT^{81, 82}. Validation and assessment of structure quality were performed using MolProbity and OneDep prior to finalization^{83, 84}. The statistics and metrics of reported structure are compiled in Table 13. Figures featuring protein structure were prepared using UCSF Chimera⁸⁵ or UCSF ChimeraX⁸⁶.

Ligand Docking with AutoDock Vina

Ligand docking was performed using the default setting⁸⁷. The determination of grid center was guided by superimposition of SbSOMT-resveratrol-β-NAD ternary complex, which the grid center was set in the binding pocket where the superimposed resveratrol is observed. For piceatannol docking in SbSOMT, the grid size was set to 18 Å×16 Å×12 Å, covering the entity of superimposed resveratrol. For docking in SbCOMT, a grid size of 24 Å×24 Å×24 Å was used with the similar grid center to cover the entire binding pocket. All docking were performed using global searching exhaustiveness of 8 and top 5 docked conformations were recorded for further analysis.

### Phylogenetic Analysis

Multiple sequence alignment was done by ClustalW and Clustal Omega with default configurations⁸⁸⁻⁸⁹. The unrooted phylogenetic trees were constructed by maximum likelihood method with 1,000 bootstrap replicates using MEGA X90.

### In Vitro Fungitoxicity Assays

A conidial suspension of Collectotrichum sublineola isolate TX430BB at a concentration of 5×104 spore ml-1 was prepared in double deionized water. Stilbenes including resveratrol, pinostilbene, and pterostilbene were dissolved in 80% (v/v) methanol and supplemented to the potato dextrose agar at concentrations ranging from 0 (mock) to 50 μM. Ten microliters of conidial suspensions were transferred onto the water agar plates, and 9 biological replicates were conducted for each concentration of stilbenes. These plates were sealed, incubated overnight at room temperature in darkness, followed by incubation under constant light for 3 days. Spore germination was observed under a Leica DM500 light microscope (Leica, USA) after incubation in dark. Mycelial growth, represented by the fungal colony diameter, was assessed after the 3-day incubation period under light using the same microscope and the ImageJ software⁹¹.

### Accession Numbers

Protein sequences analyzed in this study and used in phylogenetic analysis can be found under accession numbers listed in Table 14⁶⁵.

### Data Availability

Atomic coordinates and structure factors for the crystal structures reported in this work were deposited to the Protein Data Bank under accession numbers: 7VB8 (SbSOMT-resveratrol-β-NAD ternary complex), 7WAQ (SbSOMT-resveratrol binary complex), 7WAR (SbSOMT-pinostilbene-β-NAD ternary complex) and 7WAS (SbSOMT-pterostilbene-β-NAD ternary complex).

### Results

### Accumulation of O-Methylated Stilbenes in Pathogen-Infected Sorghum

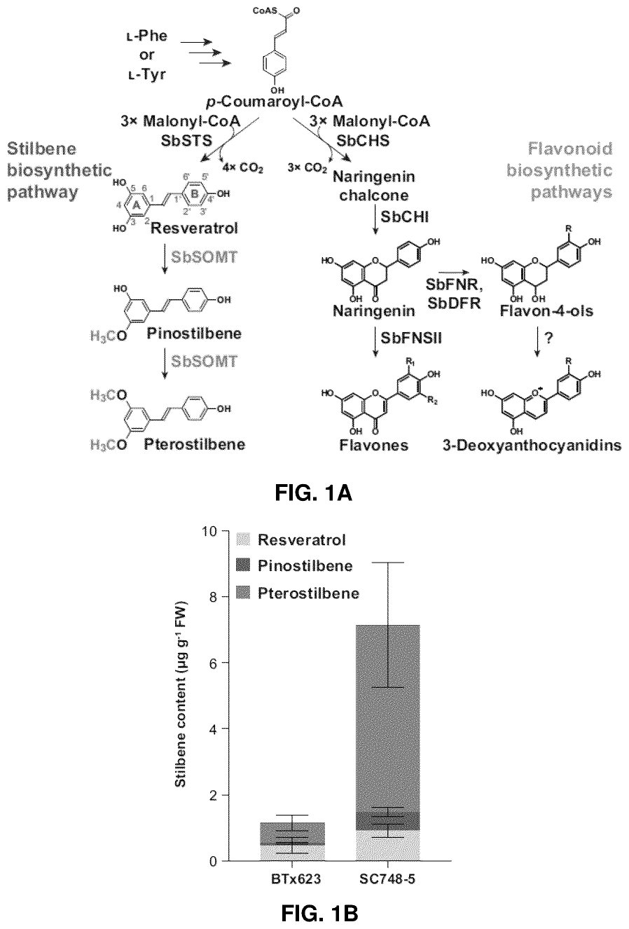

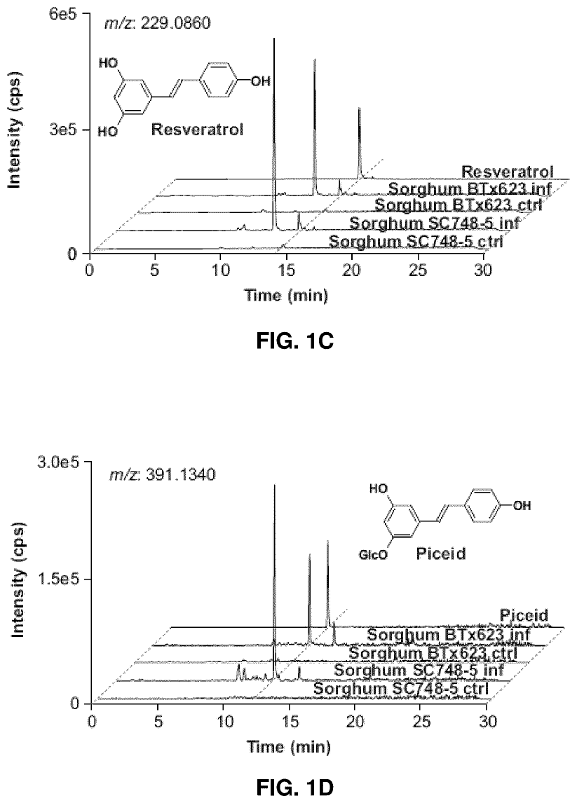

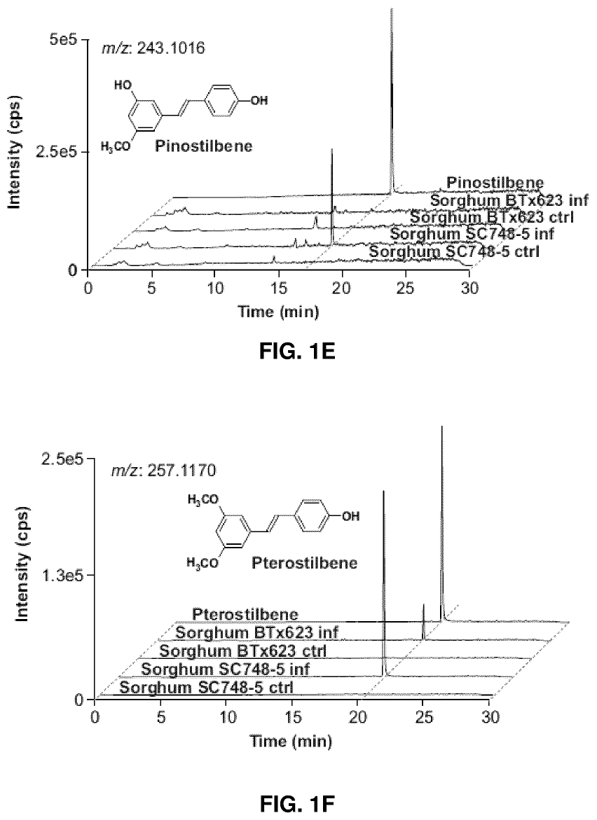

The metabolic profiles of wild-type Sorghum upon infection of Colletotrichum sublineola (FIG. 1A) were first examined. Two Sorghum genotypes, BTx623 and SC748-5, susceptible and resistant to C. sublineola infection respectively³⁷⁻³⁸, were analyzed. Accordingly, four stilbenes including resveratrol, piceid (resveratrol 3-O-glucoside), and resveratrol derivatives O-methylated at their A-ring: pinostilbene (3-O-methylated resveratrol), and pterostilbene (3,5-bis-O-methylated resveratrol) were identified in the infected Sorghum mesocotyls (FIGS. 1B-1F). In addition, flavones (apigenin, luteolin, chrysoeriol, tricin; Table 3) and 3-deoxyanthocyanidins (orange-red pigments; apigeninidin, luteolinidin, diosmetinidin; Table 4), which are known Sorghum phytoalexins, were detected³⁷⁻³⁸. Notably, the resistant genotype SC748-5 accumulated approximately 9-fold more pterostilbene than the susceptible genotype BTx623 starting from 48 h post infection (FIG. 1B & Table 5). Taken together, stilbene accumulation in SC748-5 was more rapid, and at larger quantities than BTx623 during C. sublineola infection.

### Sorghum Pathogen-Inducible SbSOMT Catalyzes Pterostilbene Biosynthesis

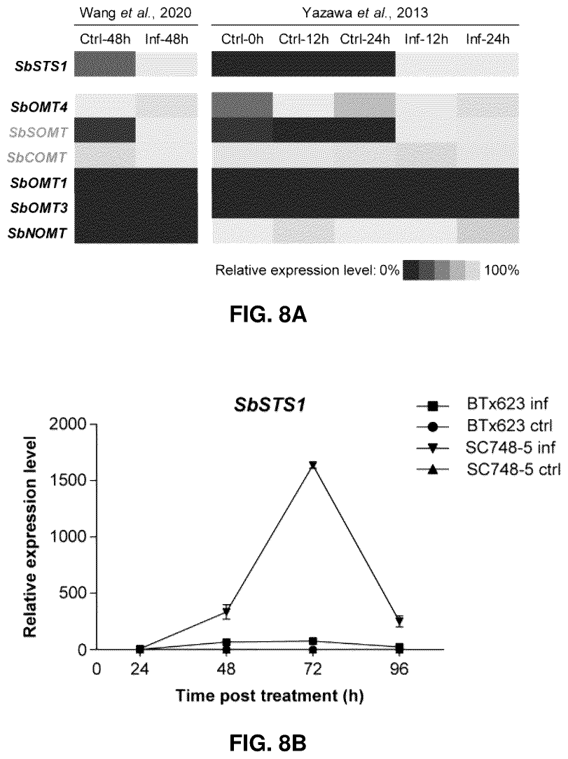

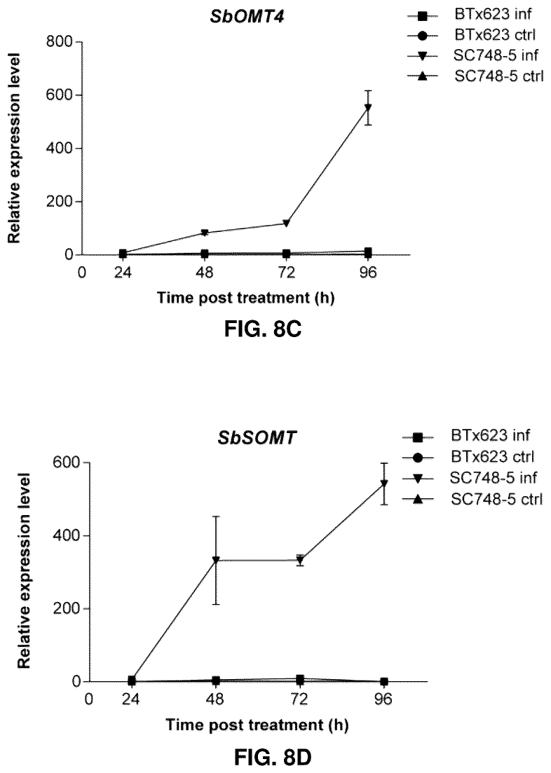

To identify potential OMTs involved in pterostilbene biosynthesis in Sorghum, an in silico expression dataset for Bipolaris sorghicola-infected Sorghum leaves (internet site matsui-lab.riken.jp/morokoshi/)³⁹⁻⁴⁰ and in-house transcriptome dataset for C. sublineola-infected Sorghum mesocotyls⁴¹ were analyzed. Transcripts of SbOMT1 and SbOMT3, which were reported to methylate resveratrol in vitro and in transgenic plants, were not detected in either dataset (FIG. 8A). Meanwhile, SbCOMT, which encodes a bona fide canonical COMT^{18, 21, 22}, is constitutively expressed in Sorghum (FIG. 8A). Furthermore, two putative OMT genes, SbSOMT (Sb07g004710) and SbOMT4 (Sb07g004690), showed pathogen-inducible expression patterns similar to that of SbSTS1 (Sb07g004700) (FIG. 8A) encoding stilbene synthase (STS) which generates resveratrol from p-coumaroyl-CoA and malonyl-CoAs (FIG. 1A)⁵. Gene expression analyses confirmed their transcriptional upregulation in both Sorghum genotypes shortly after infection (FIGS. 8B-8D). In particular, the resistant genotype SC748-5 showed stronger expression for these genes than the susceptible genotype BTx623, consistent with its higher levels of pathogen-induced stilbene accumulation.

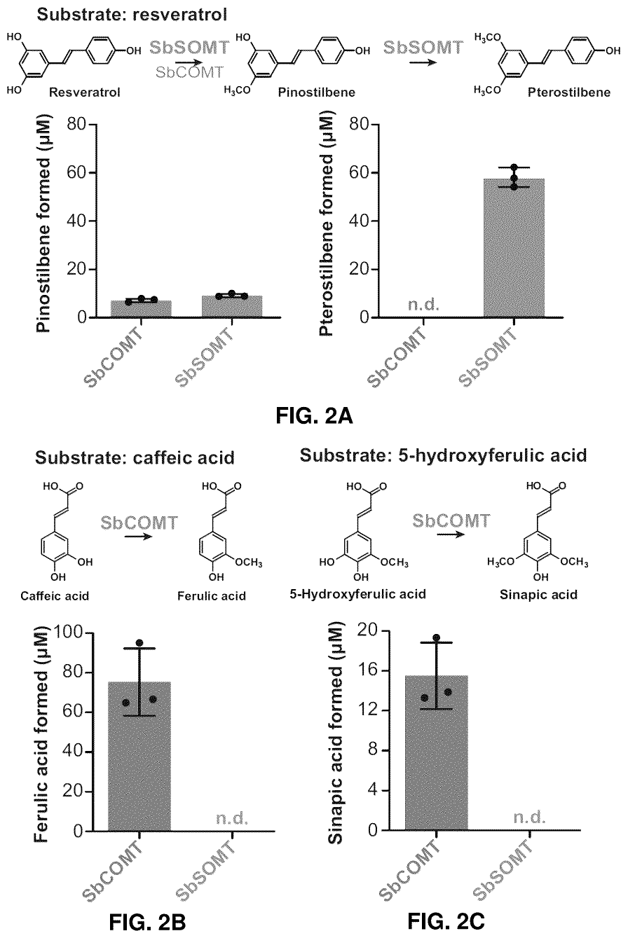

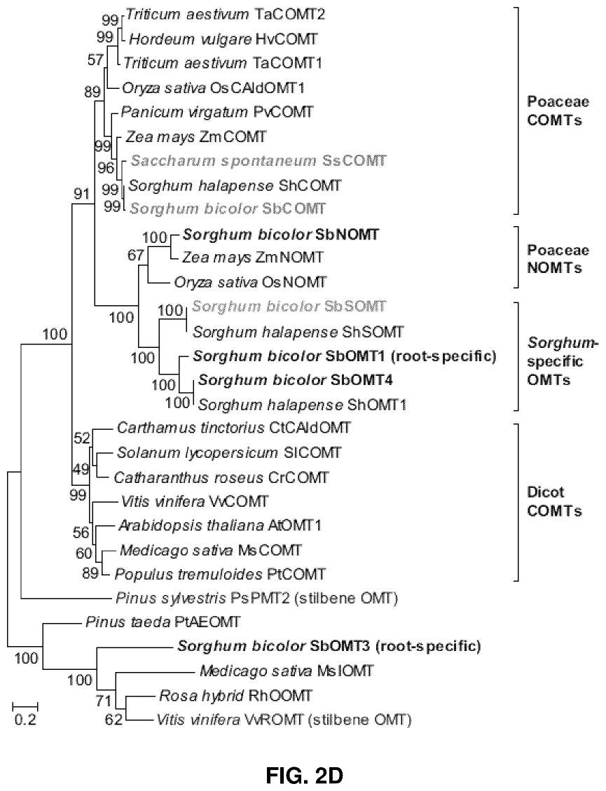

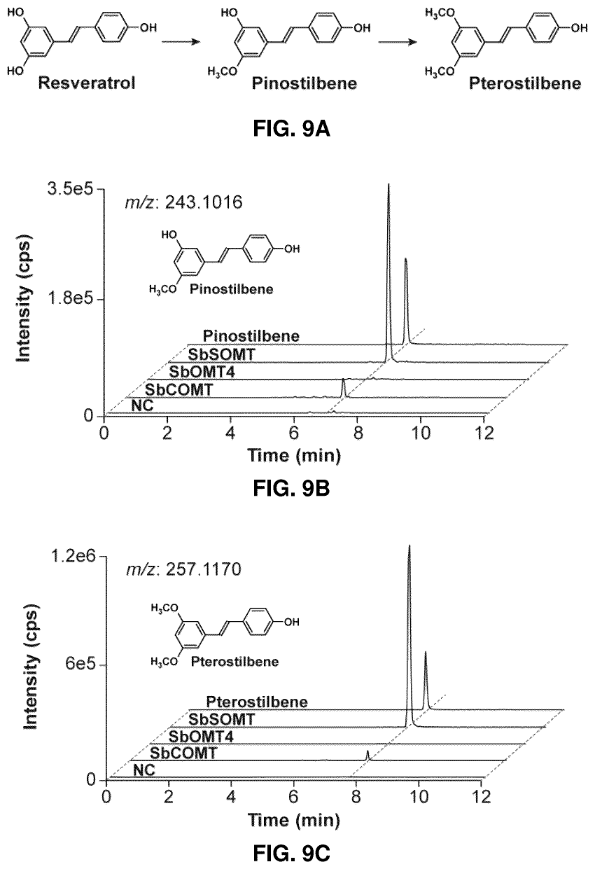

Subsequently, recombinant proteins expressed in E. coli were purified for in vitro enzyme assays. Both SbSOMT and SbCOMT generated pinostilbene and/or pterostilbene when incubated with resveratrol albeit at significantly different levels. Notably, SbSOMT converted about 60% of resveratrol into pterostilbene whereas SbCOMT displayed poor resveratrol 3,5-bis-O-methylation activities (FIG. 2A). Meanwhile, SbOMT4 showed minimal SOMT activities (FIGS. 9A-9C) and is thus unlikely to contribute to pterostilbene production in Sorghum. The catalytic activities of SbSOMT and SbCOMT towards hydroxycinnamic acids were also compared. Intriguingly, SbSOMT failed to methylate caffeic acid and 5-hydroxyferulic acid, whereas SbCOMT efficiently converted them to ferulic acid and sinapic acid, respectively (FIGS. 2B-2C).

Kinetic parameters of SbSOMT and SbCOMT were then determined using pinostilbene as a substrate since SbSOMT rapidly converts resveratrol into pterostilbene, thus preventing accurate quantification of pinostilbene produced. Results revealed the superior catalytic performance (kcat/Km and specific activity) of SbSOMT over SbCOMT and SbOMT4 towards pinostilbene, although SbSOMT showed a higher Km value (Table 1). These data indicated that SbSOMT, but not SbCOMT, is the primary SOMT for pterostilbene biosynthesis in Sorghum.

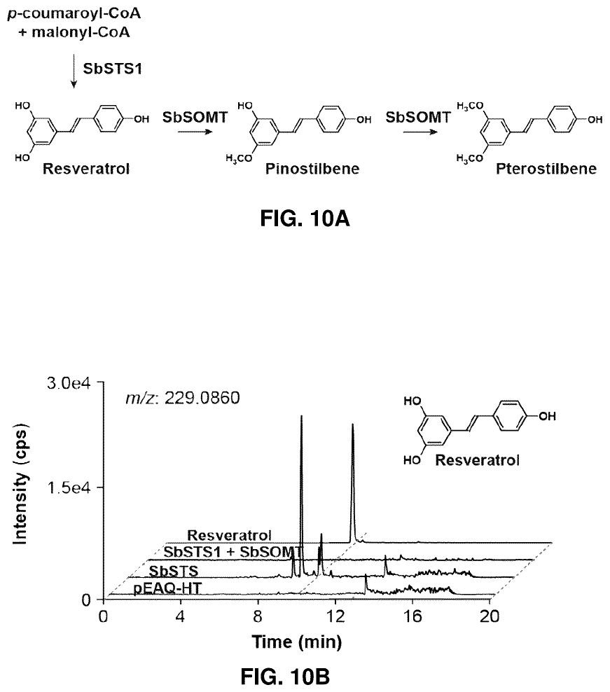

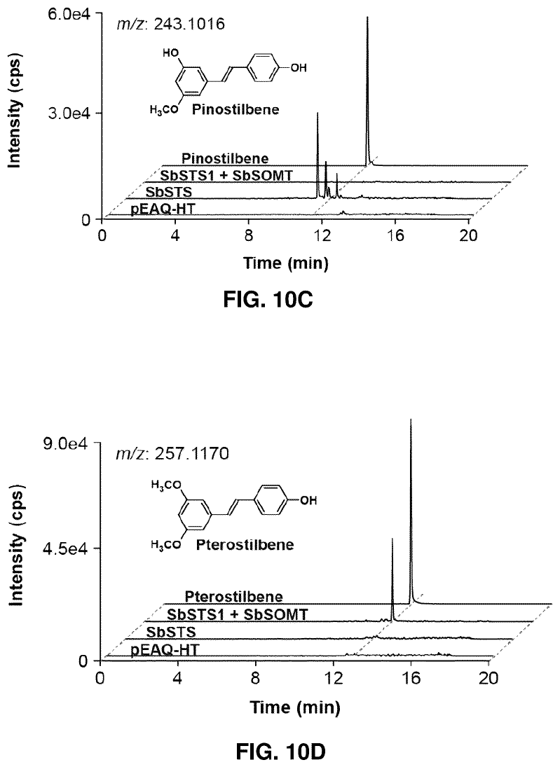

The biochemical function of SbSOMT was then examined via transient co-overexpression in Nicotiana benthamiana (FIGS. 10A-10D). Overexpression of both SbSTS1 and SbSOMT produced pterostilbene as the only stilbene product in agro-infiltrated leaves. By contrast, overexpression of SbSTS1 alone generated resveratrol and a small amount of pinostilbene, presumably due to endogenous promiscuous activities of tobacco OMT(s). These data are supportive for the role of SbSOMT in 3,5-bis-O-methylation of resveratrol to pterostilbene in planta.

### Sorghum SbSOMT Mutants are Depleted in Pathogen-Inducible Pinostilbene and Pterostilbene

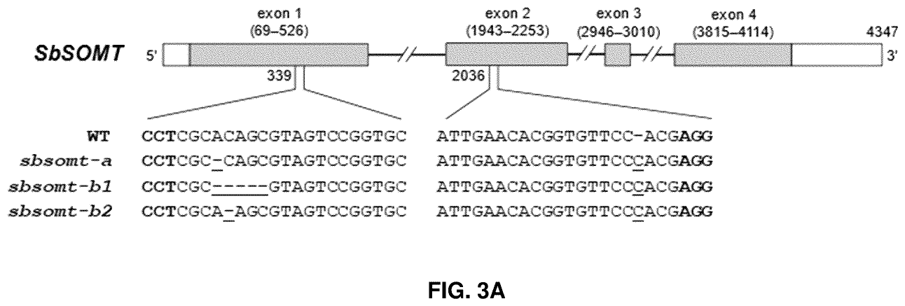

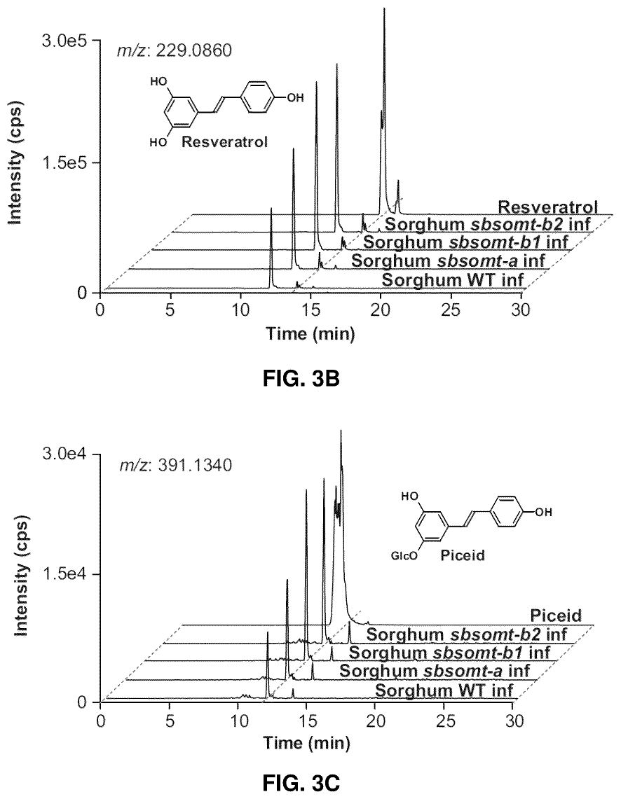

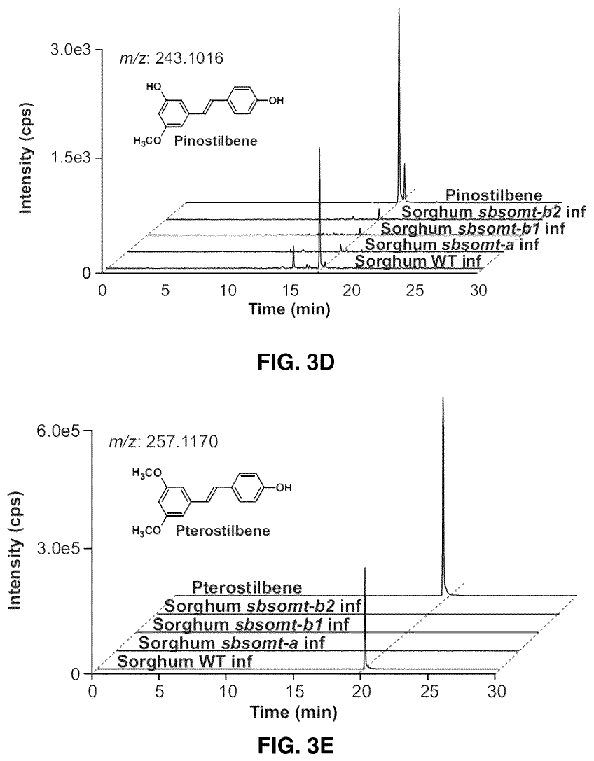

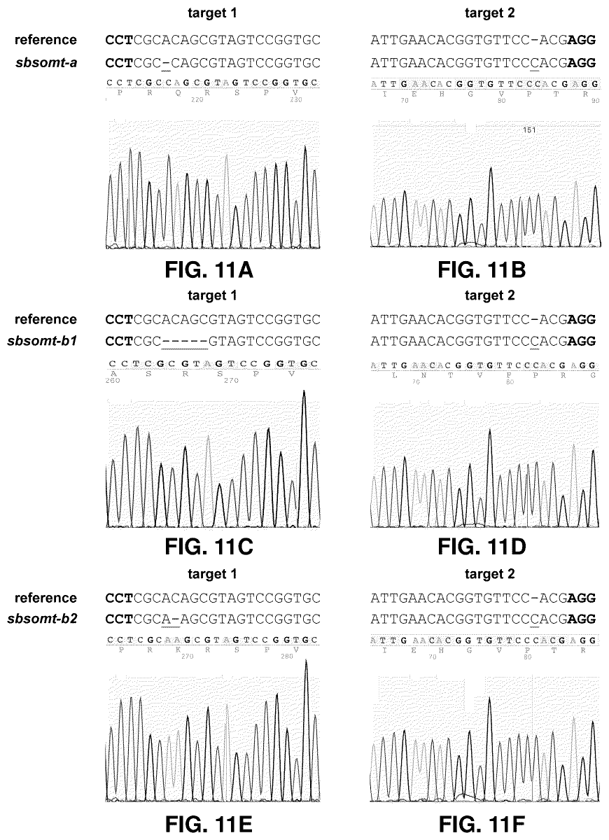

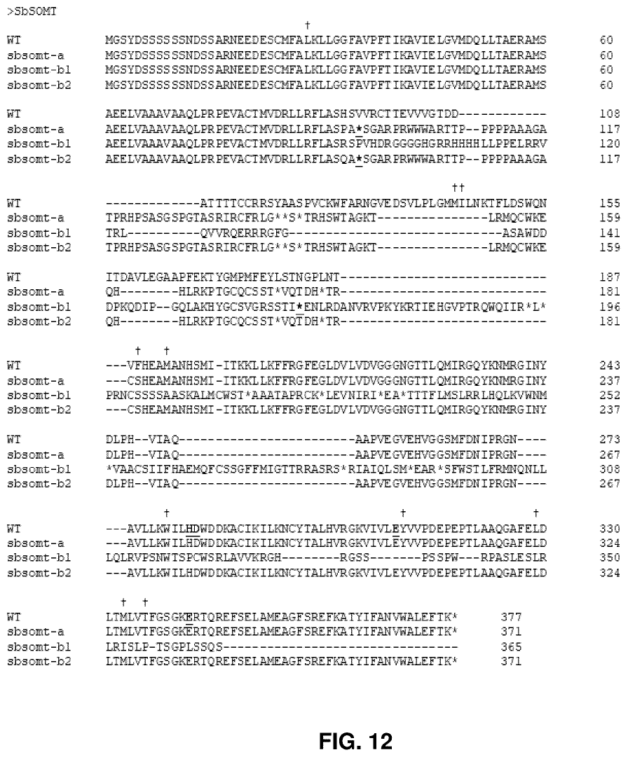

Sorghum sbsomt mutants were generated via CRISPR/Cas9-mediated genome editing. Three homozygous sbsomt mutant lines (sbsomt-a, sbsomt-b1, sbsomt-b2; T1 generation) harboring different mutation pattern were isolated for metabolite analysis (FIG. 3A & FIGS. 11A-11F). In all mutant lines, the indels on exon 1 alone were sufficient to induce frameshift mutations and premature translation termination, resulting in knockout mutations (FIG. 12).

Metabolite profiles of sbsomt mutants were analyzed upon C. sublineola infection. (FIGS. 3B-3E & Table 6). Consistent with the results above (FIGS. 1C-1F), wild-type Tx430 mesocotyls accumulated resveratrol, piceid, pinostilbene and pterostilbene 72 h after infection. By contrast, the sbsomt-a/b1/b2 mutant mesocotyls accumulated resveratrol and piceid but not pinostilbene or pterostilbene even at 96 h after infection. These results established the indispensable role of SbSOMT in 3,5-bis-O-methylation of resveratrol to form pinostilbene and pterostilbene successively in Sorghum.

### Sorghum Convergently Recruited SbSOMT Via Neofunctionalization of COMT

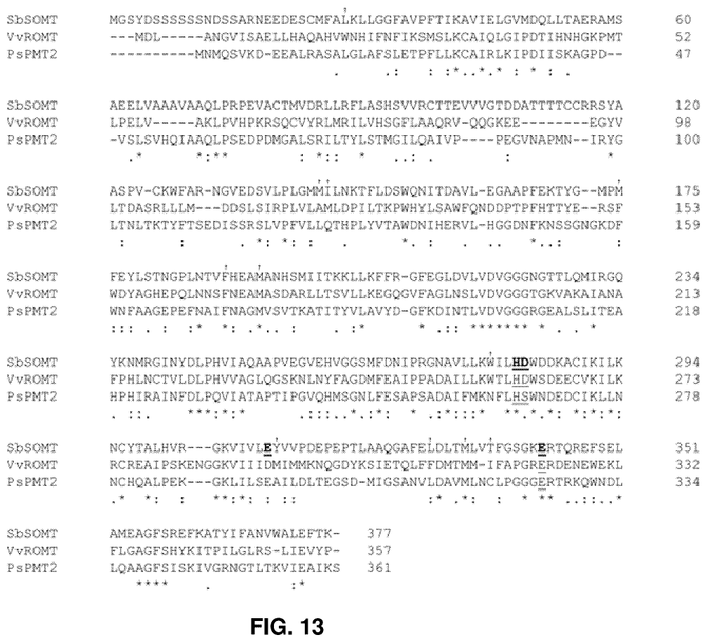

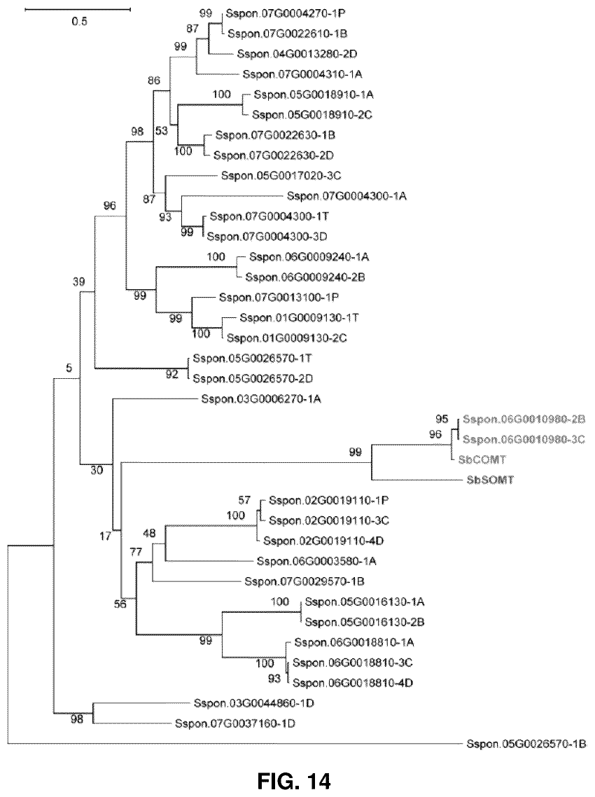

It has been discovered that SOMTs in grapevine (VvROMT) and Scots pine (PsPMT2) evolved convergently from non-COMT ancestors^{27, 28}. Multiple sequence alignment showed that SbSOMT shares only 31.9% and 29.9% sequence identity with VvROMT and PsPMT2, respectively (FIG. 13). Instead, SbSOMT is phylogenetically related to canonical COMTs in Poaceae (FIG. 2D). In addition, an SbSOMT ortholog (ShSOMT) is found in Johnsongrass (Sorghum halapense; FIG. 2D). Meanwhile, no potential SbSOMT orthologs could be retrieved from wild sugarcane (Saccharum spontaneum) proteome (internet site plants.ensembl.org/; all homologous proteins with <50% identity; FIG. 14) although Sorghum spp. and Saccharum spp. share a recent common ancestor (both belonging to the same subtribe). Furthermore, ShOMT1, ShSOMT, SbOMT1, SbOMT4, SbSOMT form a Sorghum-specific clade sister to Poaceae naringenin 7-O-methyltransferases (NOMTs) (FIG. 2D). Hence, these OMTs were most likely recruited via duplication and neofunctionalization of an ancestral grass COMT. Overall, SbSOMT represents an independent and genus-specific acquisition of SOMT activities distinct from those in grapevine and Scots pine.

### Substrate Binding Affinity and Regioselectivity of SbSOMT and SbCOMT

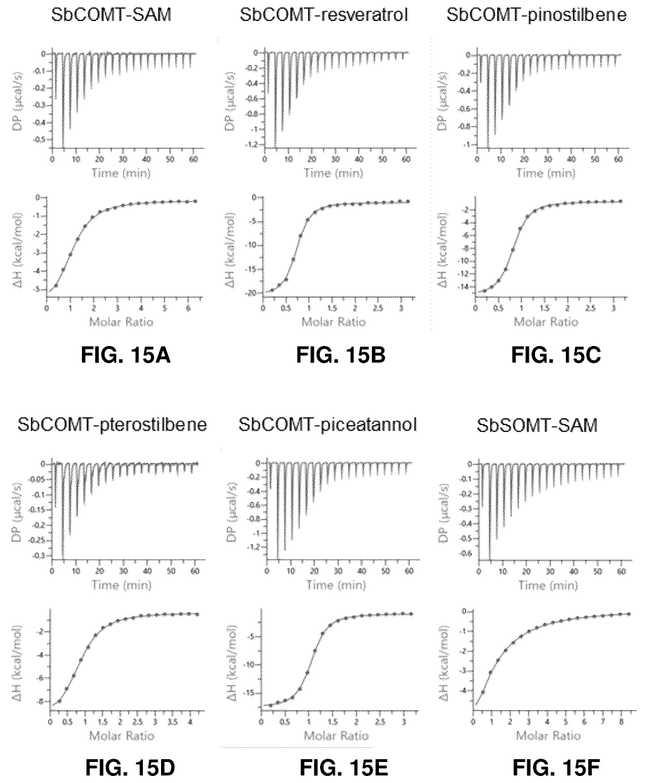

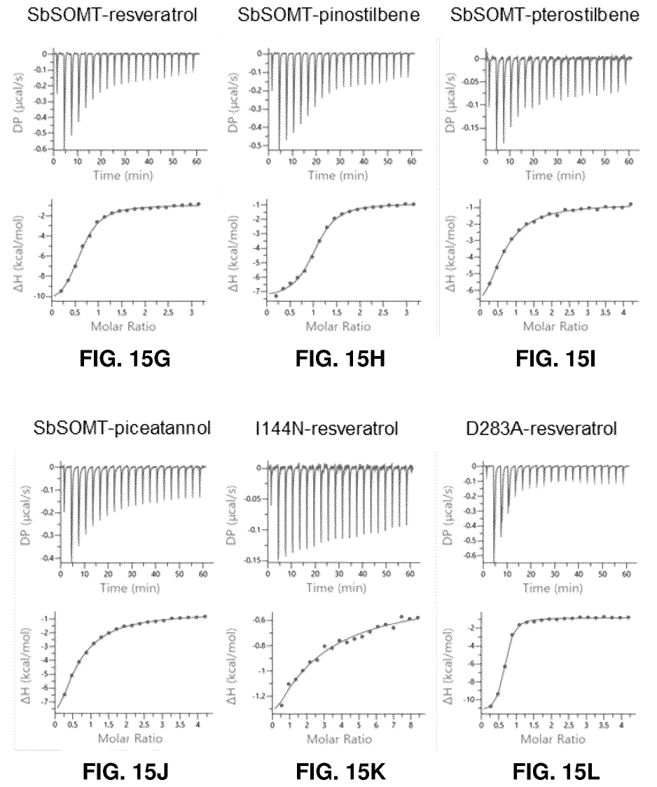

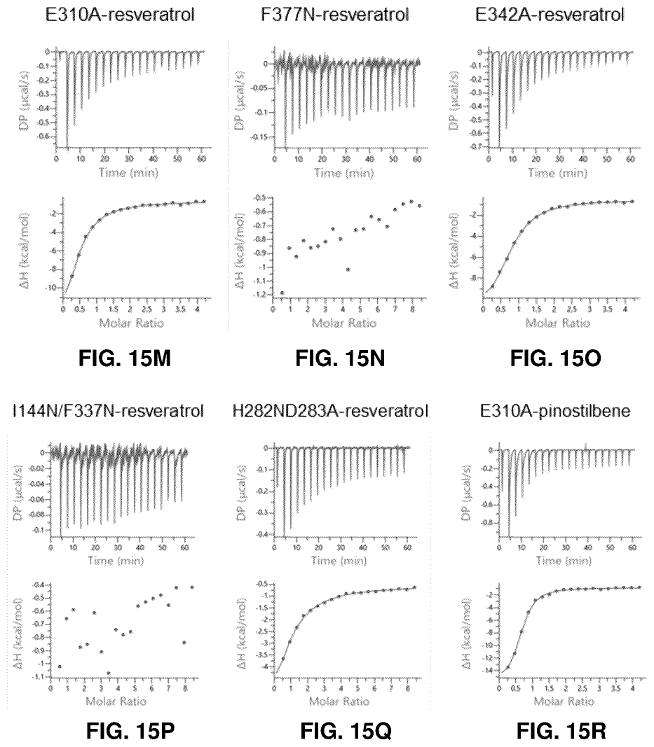

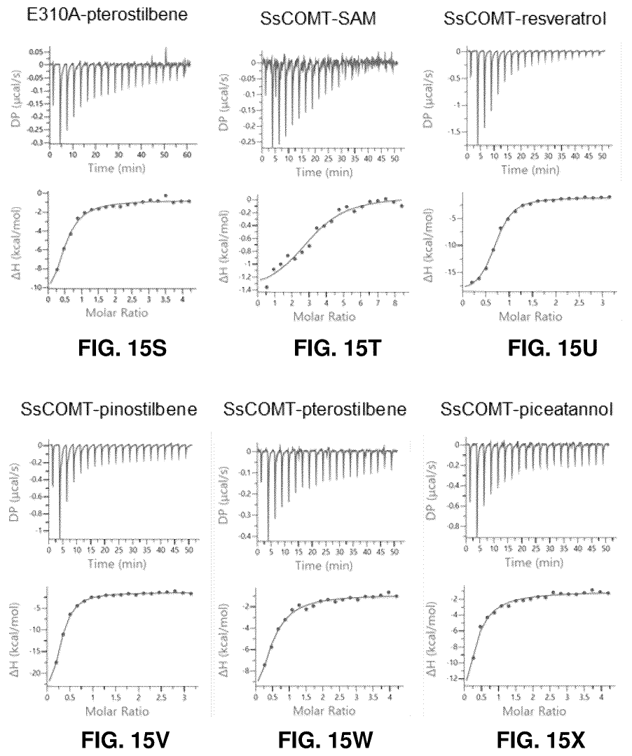

So far, the results presented herein revealed that SbSOMT catalyzes the 3,5-bis-O-methylation of resveratrol upon fungal infection. Meanwhile, SbCOMT efficiently methylates hydroxycinnamic acids but demonstrates minimal SOMT activities towards resveratrol and pinostilbene which might be attributed to poor substrate binding. However, isothermal titration calorimetry (ITC) study did not corroborate this possibility (Table 2 & FIG. 15). In fact, SbCOMT exhibited strong, micromolar binding affinities (Kd) towards resveratrol (1.55 μM), pinostilbene (1.79 μM), and pterostilbene (3.99 μM), comparable to those towards hydroxycinnamic acids²¹. Meanwhile, SbSOMT also showed strong binding affinities towards resveratrol and pinostilbene (4.69 μM and 2.86 μM respectively) but a higher Kd to pterostilbene (11.20 μM), demonstrating SbSOMT favors its substrates over product for binding. The SAM binding affinity of SbSOMT was unexpectedly weak (Kd=90.00 μM), whereas SbCOMT showed a Kd of 13.00 μM which is consistent with a previous study²¹. Collectively, SbSOMT and SbCOMT showed comparable stilbene-binding affinities, which would not account for their different catalytic activities towards resveratrol and pinostilbene.

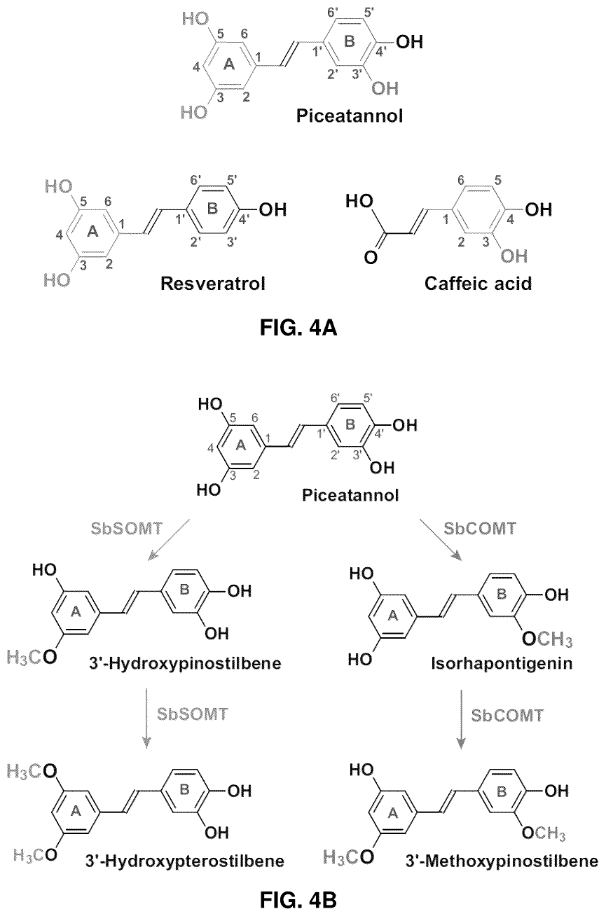

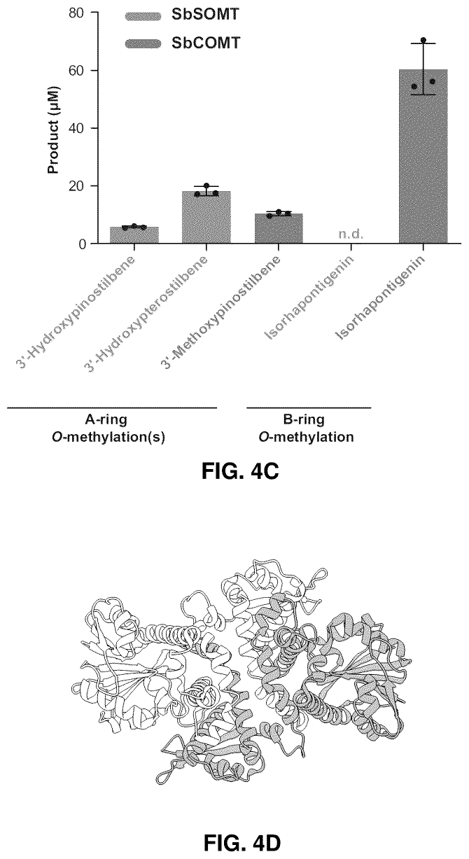

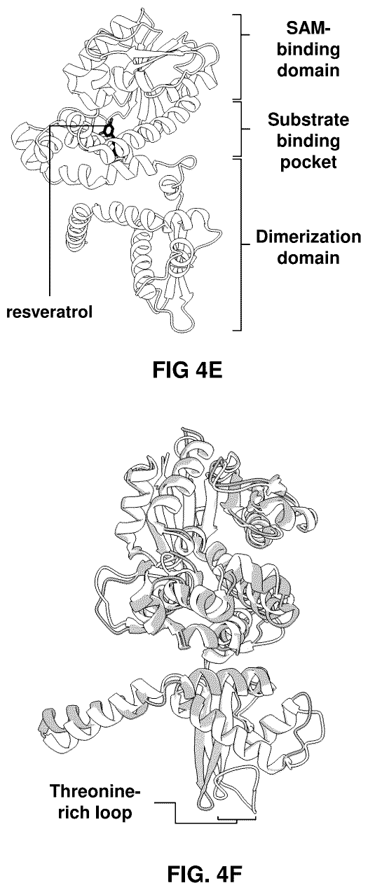

Canonical COMTs including SbCOMT utilize the para-hydroxyl group of phenylpropanoids for proper substrate orientation^{21, 22} which concurs with results obtained herein of SbCOMT-catalyzed O-methylation of caffeic acid and 5-hydroxyferulic acid (FIG. 2B). On the other hand, SbSOMT-catalyzed O-methylation of resveratrol occurs in the A-ring which is not para-hydroxylated. Piceatannol (3′-hydroxylated resveratrol) was then used as an enzyme substrate since its stilbene B-ring is structurally identical to the phenolic ring in caffeic acid while its A-ring is identical to that in resveratrol (FIG. 4A). Both enzymes catalyzed in vitro O-methylation of piceatannol, but occurring in a regioselective manner (FIG. 4B-4C). SbSOMT methylated the 3- and 5-hydroxyl groups on the piceatannol A-ring to produce 3′-hydroxypinostilbene and 3′-hydroxypterostilbene successively. By contrast, SbCOMT converted a substantial amount of piceatannol (ca. 60%) into isorhapontigenin by 3′-O-methylation in the B-ring with a conversion rate similar to those of SbCOMT-caffeic acid and SbSOMT-resveratrol reactions (FIG. 2A-2B). A small amount of isorhapontigenin was further methylated at its A-ring to 3′-methoxypinostilbene by SbCOMT (FIG. 4B-4C) and the conversion rate was similar to that of SbCOMT-resveratrol reaction (FIG. 2A). Furthermore, ITC experiments showed that SbCOMT displayed significantly stronger binding affinity towards piceatannol when compared to SbSOMT (Table 2; Kd=1.26 μM and 37.50 μM, respectively). Overall, these experiments strongly demonstrated the different regioselective O-methylation properties of SbSOMT and SbCOMT.

Global Structure of SbSOMT Resembles that of SbCOMT

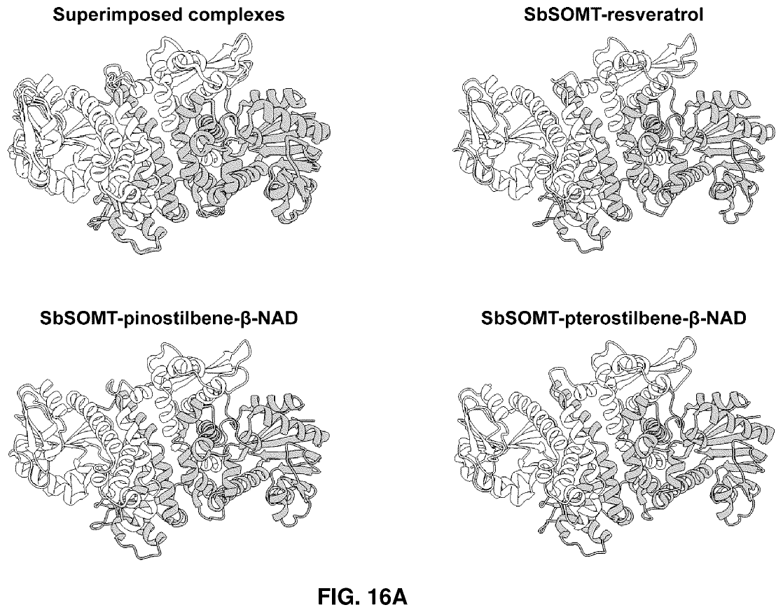

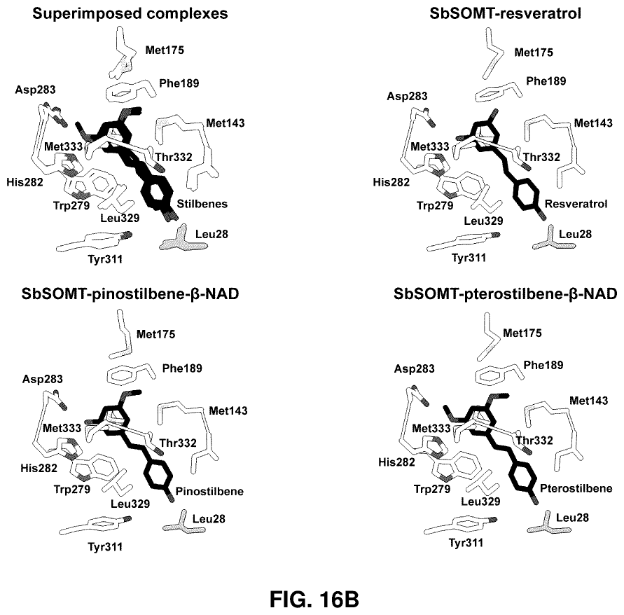

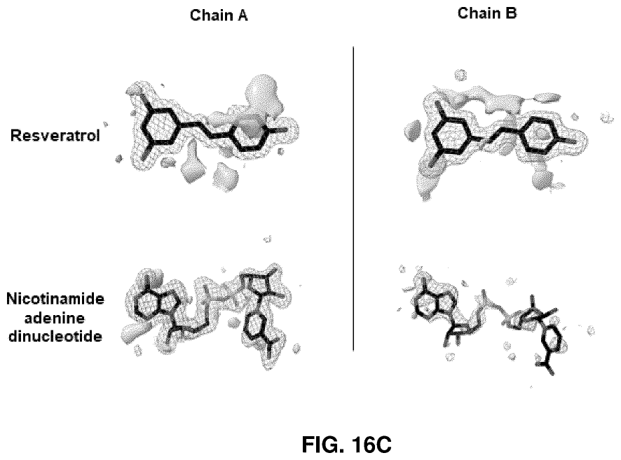

To rationalize their different catalytic regioselectivities, structural analyses involving X-ray crystallography and computational methods were conducted. The crystal structure of an SbSOMT-resveratrol-nicotinamide adenine dinucleotide (β-NAD) ternary complex was solved (diffracted to 1.72 Å resolution, FIG. 4D; β-NAD was utilized as an additive to improve crystallization⁴²), an SbSOMT-resveratrol binary complex, and two ternary complexes reproduced with pinostilbene or pterostilbene (diffracted in the range of 2.10-2.56 Å, FIG. 16A). All complexes were in high structural resemblance and depicted as a homodimer with an open conformation. Superimposition of SbSOMT and SbCOMT (PDB: 4PGH²¹) revealed their high structural similarity (RMSD=2.322 A), except that SbSOMT harbors an extended loop (Val103-Cys114) that forms a threonine-rich dimerization interface (FIG. 4D).

Structural Analyses and in Silico Docking Support Different Stilbene Orientations within the Substrate Binding Pockets of SbSOMT and SbCOMT

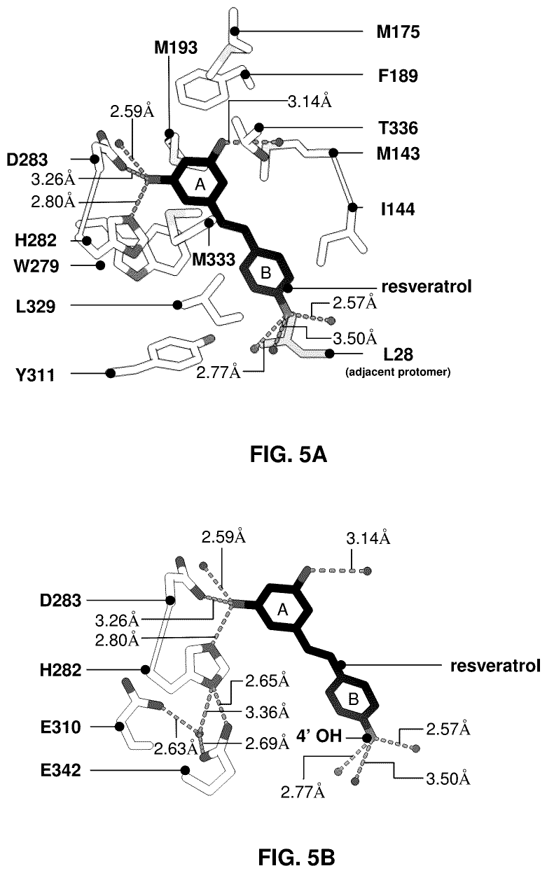

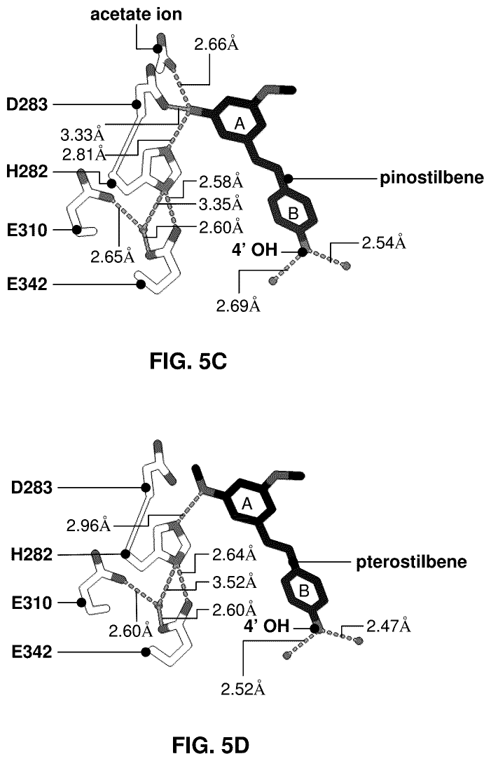

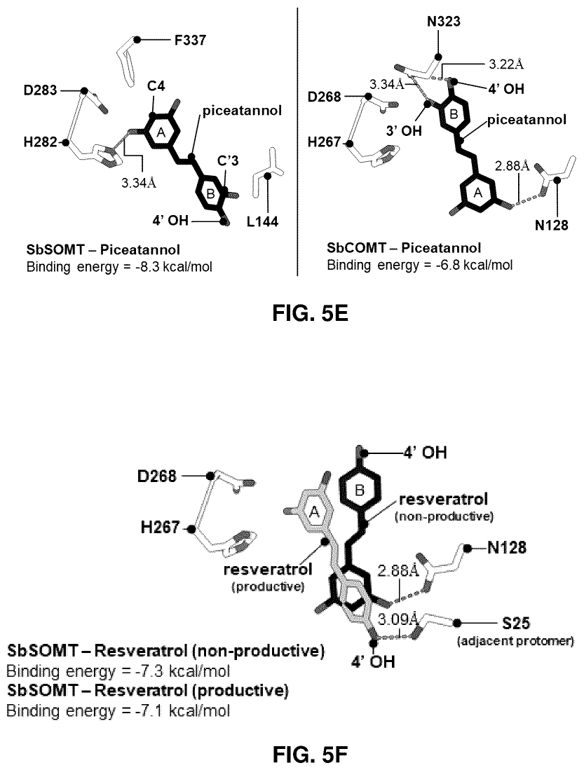

Close examination of the SbSOMT substrate binding pocket within the SbSOMT-resveratrol-β-NAD complex provided further insights into the interactions between SbSOMT and its stilbene substrates. The pocket mainly composed hydrophobic residues, and the stilbene backbone was secured by hydrophobic interactions with Met143, Ile144, Met175, Phe189, Met193, Trp279, Tyr311, Leu329, Met333, Thr336, in addition to Leu28 of the adjacent protomer (FIG. 5A). The apparent binding cavity was confined to fit the stilbene backbone in a planar configuration with minimal rotational flexibility. Of the three hydroxyl groups on resveratrol capable of forming hydrogen bonds, only the methyl-accepting 3-OH group (A-ring) formed direct hydrogen bonds to SbSOMT residues (FIG. 5B), including the catalytic residues His282 (2.80 Å) and Asp283 (3.26 Å). Other hydroxyl groups were shielded by water molecules and did not directly interact with SbSOMT. The same binding mode was adopted for pinostilbene or pterostilbene in the corresponding SbSOMT co-crystals (FIG. 5C-5D & FIG. 16B). Overall, these structures depicted a productive substrate orientation within the substrate binding pocket which greatly favors A-ring O-methylation. Following 3-O-methylation of resveratrol, the pinostilbene intermediate will need to be dissociated and re-inserted in order to re-position the A-ring for 5-O-methylation to produce pterostilbene. Similarly, in silico docking of piceatannol with SbSOMT (FIG. 5E) conformed to such substrate orientation, with the A-ring positioned in proximity to the catalytic residues of SbSOMT, hence favoring the 3,5-bis-O-methylation to produce 3′-hydroxypinostilbene and 3′-hydroxypterostilbene (FIG. 4B-4C).

Docking stilbenes with SbCOMT (PDB: 4PGH²¹) revealed a contrasting substrate orientation to that of SbSOMT, suggesting that SbCOMT preferentially positions stilbenes with the B-ring close to the catalytic residues and the A-ring reaching inwardly into the pocket. SbCOMT-piceatannol docking showed a single favorable orientation of piceatannol among five hits which were docked at a sub-optimal position (FIG. 5E). In the top hit, the B-ring 3′- and 4′-hydroxyl groups were stabilized by hydrogen bonds with Asn323 while A-ring 5-hydroxyl group interacted with Asn128 (FIG. 5E). Consistent with the enzyme assay results (FIG. 2A), the 3′-hydroxyl group on the B-ring was orientated close to the catalytic residues and would thus predominantly favors the methylation of 3′-hydroxyl group (B-ring) over 3-/5-hydroxyl groups (A-ring) (FIG. 5E). Additionally, docking resveratrol with SbCOMT revealed two opposite orientations with similar free energies of binding (FIG. 5F), representing two competing binding modes which hampered its catalytic performance (FIG. 2A). In fact, the non-productive orientation with the B-ring closer to the catalytic residues was more energetically favorable (−7.3 kcal/mol, in blue), while the productive orientation with A-ring closer to the catalytic residues was slightly less stable (−7.1 kcal/mol, in grey). Collectively, these analyses fully corroborated with results of the biochemical assays and stilbene profiles of Sorghum presented herein, and provided a mechanistic rationale for the apparent differences in catalytic activity between SbCOMT and SbSOMT. Additionally, as the methyl-accepting —OH groups in piceatannol and hydroxycinnamic acids are both adjacent to a para-OH group (i.e., 4′-OH in piceatannol and 4-OH in hydroxycinnamic acids), this may be a prerequisite for efficient O-methylation by COMTs as suggested previously^{21, 22}.

### Sbsomt and SbCOMT Employ the Same Catalytic Residues but Different Substrate Binding Orientations

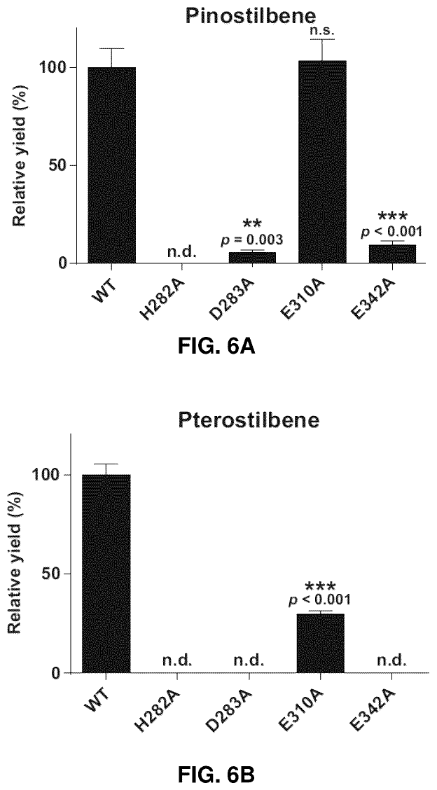

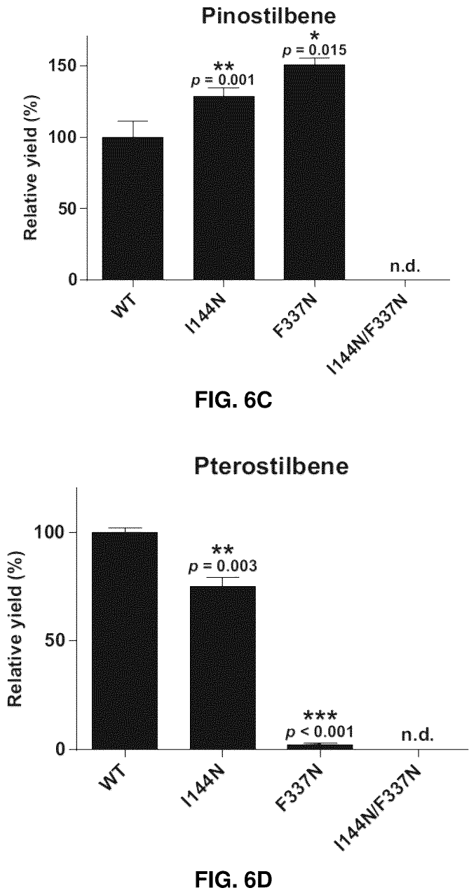

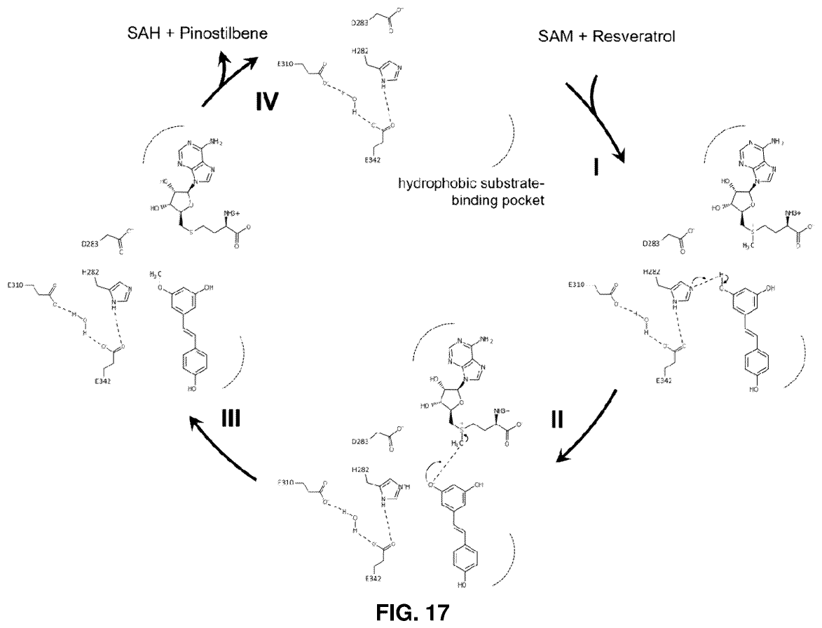

The catalytic residues of SbSOMT are highly conserved with all reported COMTs, including His282 as the key nucleophile, whereas Asp283, Glu310, and Glu342 collectively charge the basicity or confine His282 in its reactive conformation (FIG. 5B)^{21, 22, 43, 44}. Accordingly, the surrounding electrostatic force allows NF of His282 to deprotonate the methyl-accepting —OH group, followed by a SN2 nucleophilic attack of the methyl-donor group on SAM by the oxyanion (—O—) (FIG. 17)^{21, 22, 43, 44}. The roles of His282 and Asp283 have been validated by mutagenesis, but not for those of Glu310 and Glu342 (PDB: 4PGG²¹, 5ICG⁴⁵, 1KYZ⁴⁴, and 6I5Z⁴⁶). Here, H282A, D283A, E310A, and E342A mutant proteins of SbSOMT were generated for catalytic and binding assays with resveratrol. H282A mutant protein was catalytically defective and unstable for ITC study (FIGS. 6A-6B). To further elucidate the role of His282 in resveratrol binding (FIG. 5B), a stable SbSOMT H282N/D283A double mutant protein was generated. While the D283A protein showed a slightly stronger affinity towards resveratrol (Kd=1.31 μM), the H282N/D283A mutant protein showed a significantly weaker affinity (Kd=32.90 μM), implying that His282 plays a moonlighting role in substrate binding in SbSOMT in addition to catalysis (Table 7). Meanwhile, D283A and E342A mutations resulted in significant reduction in pinostilbene yield by 94.7% and 91.9%, respectively (FIG. 6A). In addition, both of them failed to generate pterostilbene when incubated with resveratrol despite that they showed considerably stronger affinities to resveratrol (FIG. 6B & Table 7). Unexpectedly, E310A mutation only caused reduction of pterostilbene yield (by 70.1%), accompanying with reshuffled stilbene binding affinities (FIG. 6B & Table 7). It was reasoned that the E310A mutation decreased the rigidity and basicity of His282, thus weakening SbSOMT selectivity towards the reactive hydroxyl group against the inactive methoxy group, which is a vital feature allowing the subsequent O-methylation of the same stilbene substrate (FIGS. 5B-5D).

In SbCOMT, two key amino acid residues, Asn128 and Asn323, cooperatively mediate the productive orientation for catalysis²¹. Docking piceatannol with SbCOMT revealed that Asn323 forms a hydrogen bond with the para-OH group on the stilbene B-ring whose meta-OH group is positioned optimally for methylation, while Asn128 likely serves to stabilize their interaction via another hydrogen bond with 5-OH on the non-para-hydroxylated stilbene A-ring (FIG. 5E). In striking contrast, the equivalent positions in SbSOMT are occupied by two highly hydrophobic residues, Ile144 and Phe337, which are unfavorable for interactions with the para-OH group of an aromatic ring as predicted by in silico docking (FIG. 5E). On the other hand, these residues may contribute to accommodate the stilbene A-ring, which is not para-hydroxylated, for methylation of its meta-hydroxyl groups. I144N, F337N and I144N/F337N SbSOMT mutant proteins were then generated for catalytic and binding assays. All three SbSOMT mutant proteins showed significantly weakened binding for resveratrol, with F337N and I144N/F337N mutant proteins displaying undetectable binding affinities (Table 7). Correspondingly, both I144N and F337N produced substantially less pterostilbene (24.8% and 97.7% respectively), and accumulated slightly more pinostilbene when compared to the WT protein (28.4% and 50.8% respectively; FIGS. 6C-6D). Meanwhile, the I144N/F337N mutant protein showed complete abolishment of SOMT activities (FIGS. 6C-6D). As pterostilbene represents the major product generated by SbSOMT-resveratrol reactions (FIG. 2A), these results established the important role of Ile144 and Phe337 in the resveratrol binding mechanism adopted by SbSOMT.

### A Canonical COMT Likely Catalyzes Isorhapontigenin Biosynthesis in Wild Sugarcane

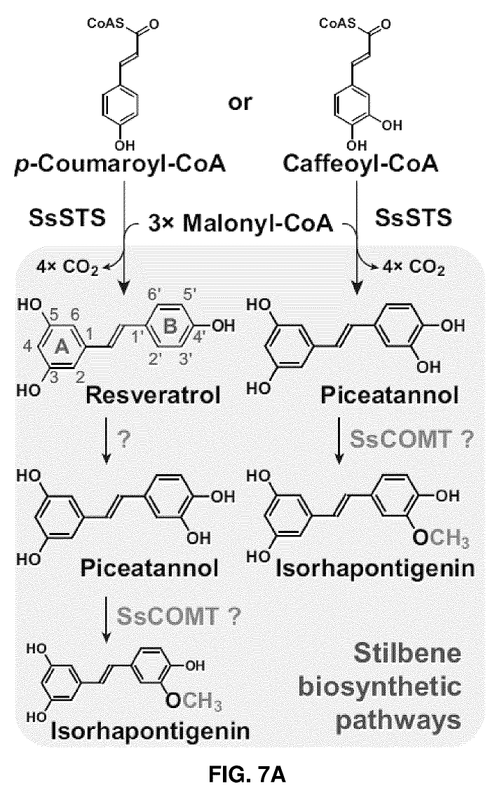

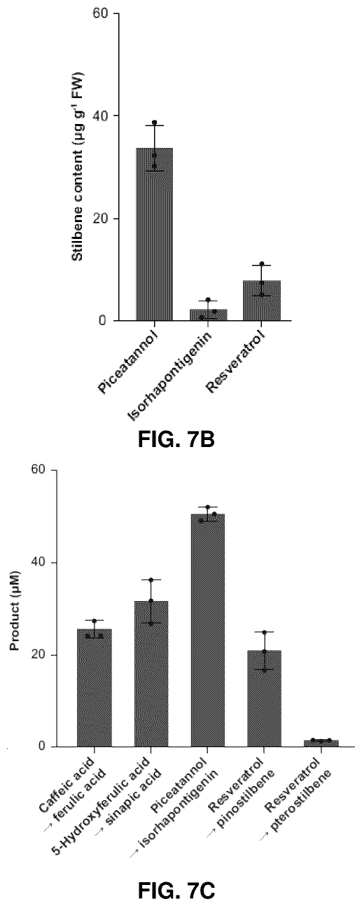

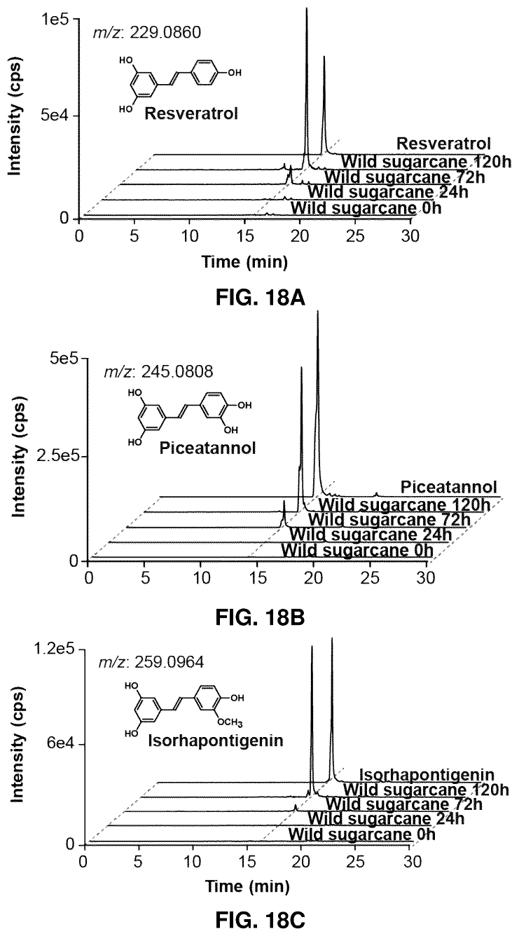

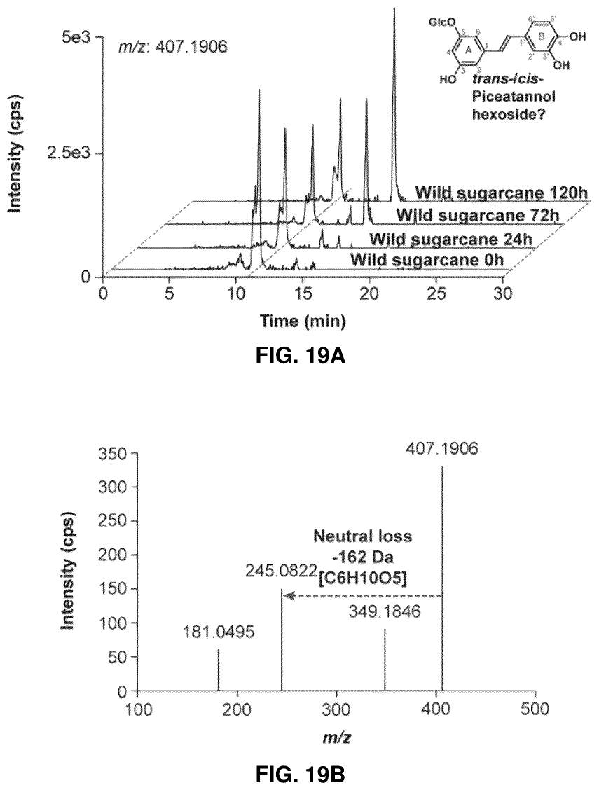

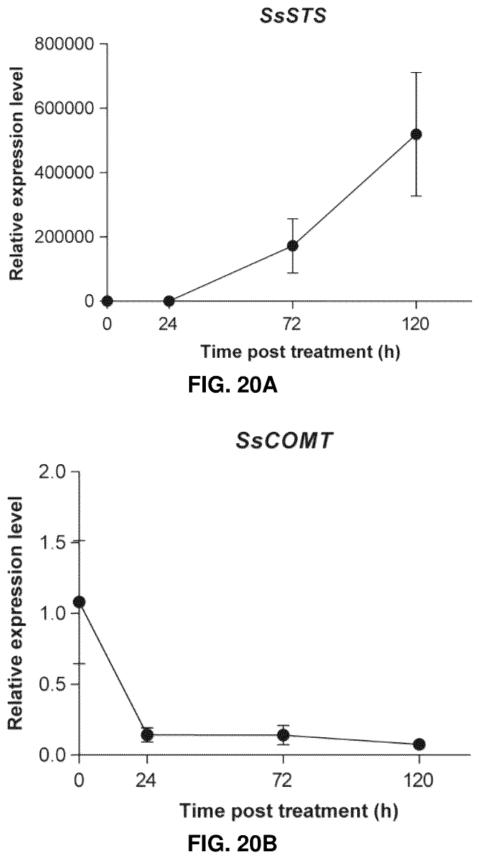

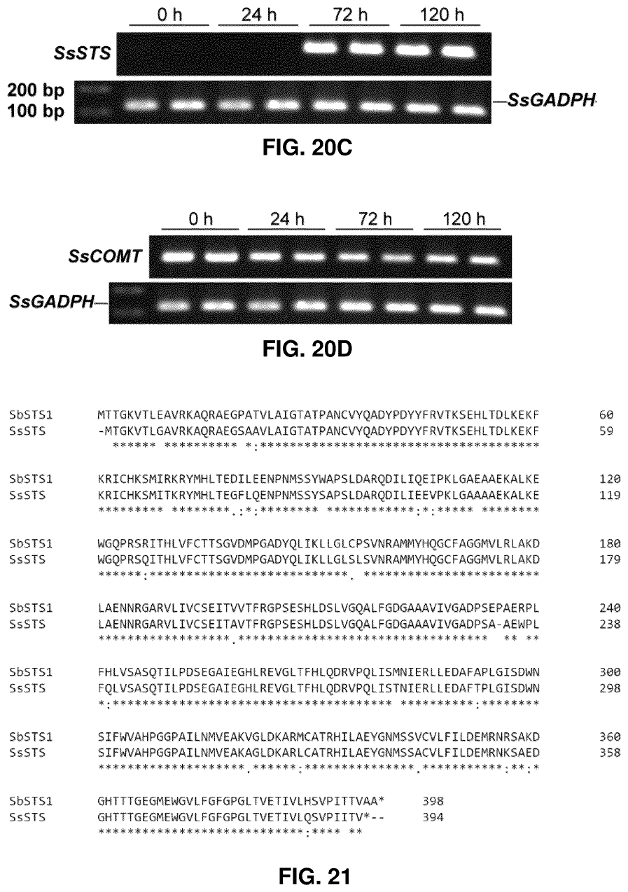

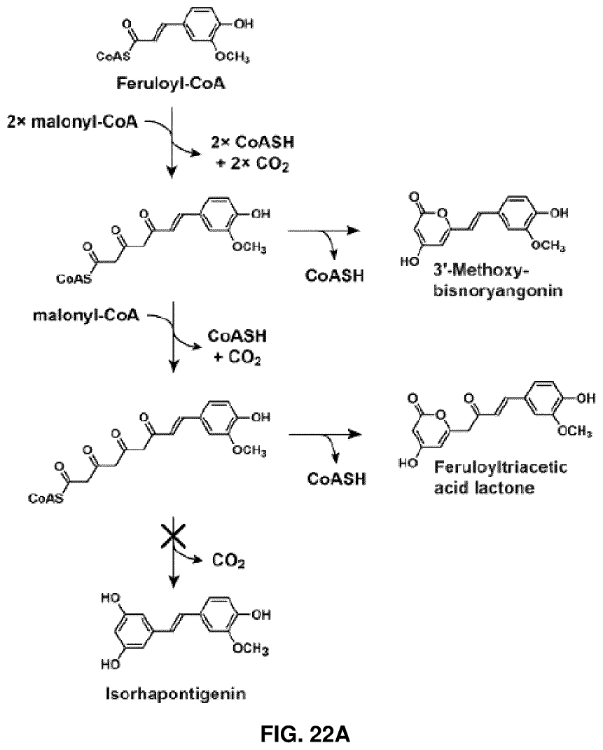

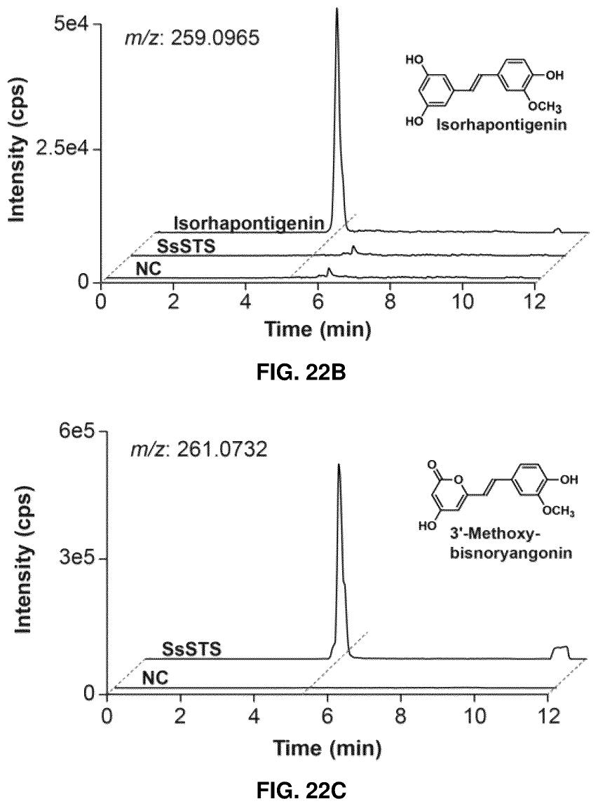

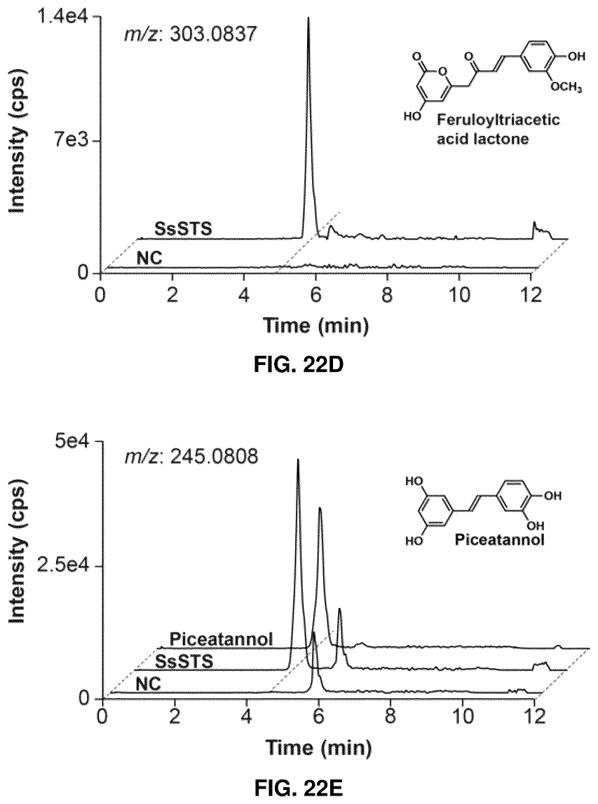

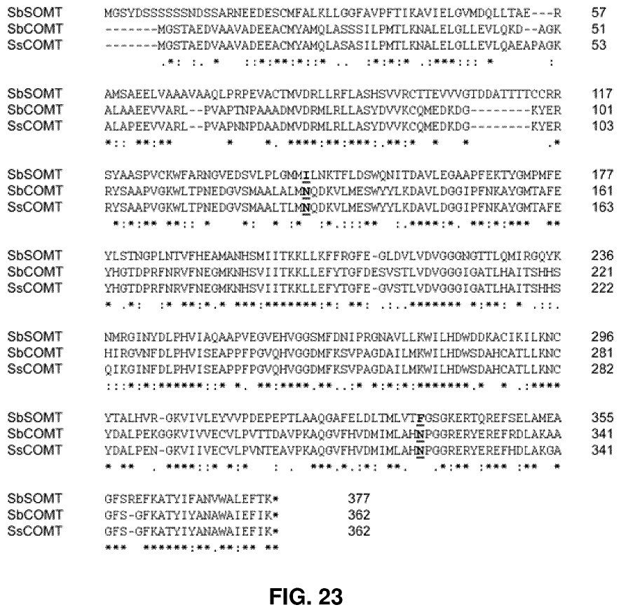

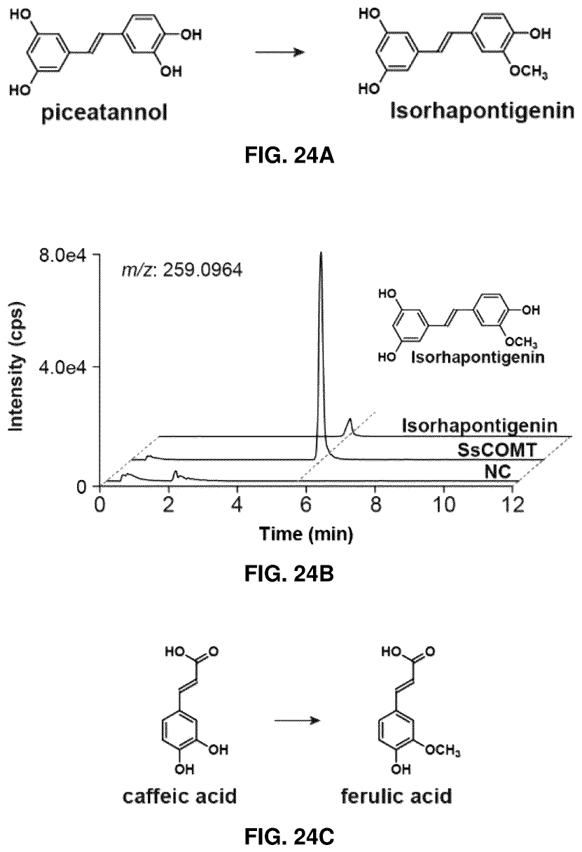

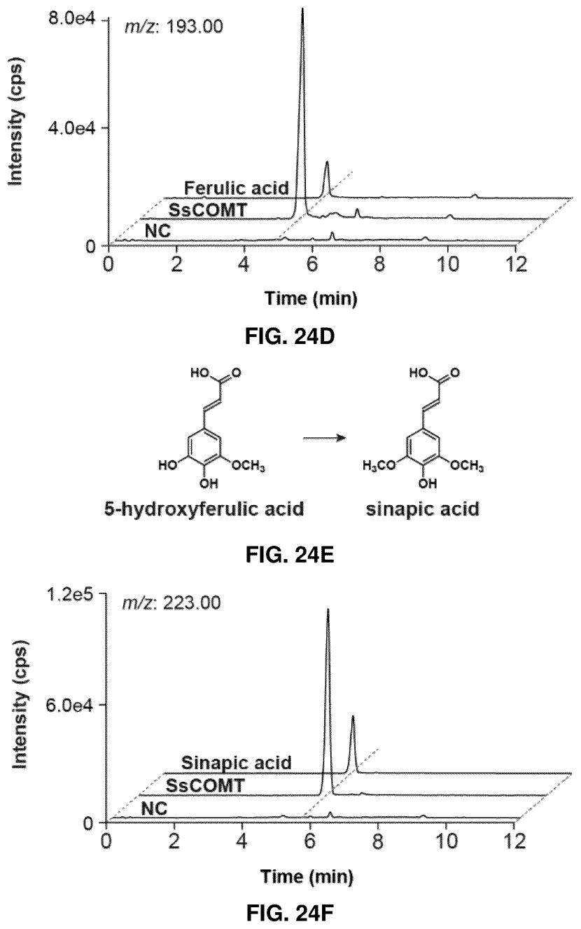

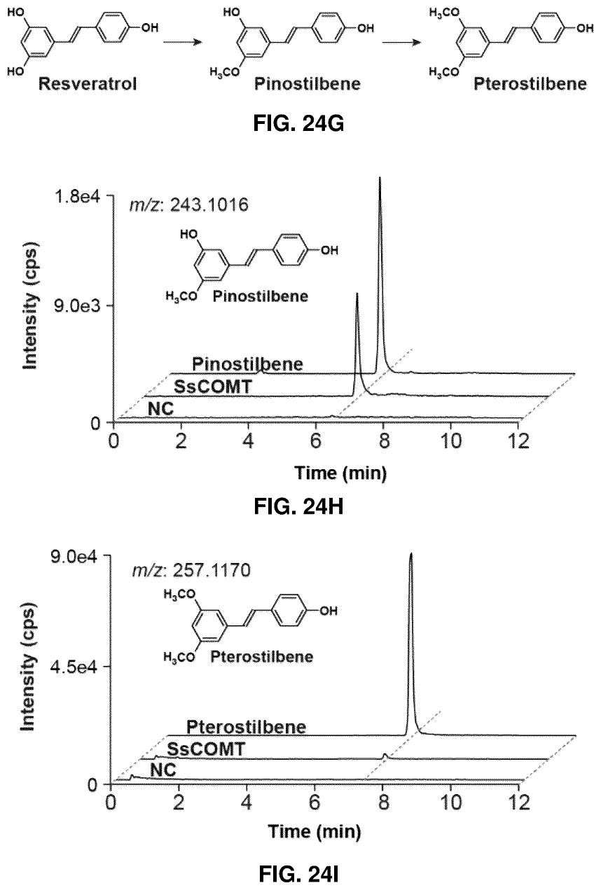

Saccharum spp. were reported to accumulate resveratrol and piceatannol (3′-hydroxylated resveratrol) upon mechanical wounding or fungal infection^{2, 36} (FIG. 7A). As Saccharum spp. are Saccharinae grasses like Sorghum spp., it was investigated whether O-methylated derivatives could also be detected in wild sugarcane whose genome is available⁴⁷. The slender stalks of flowering wild sugarcane (S. spontaneum) were sawn into 2-cm segments (excluding nodes) for metabolic profiling. No stilbenes were detected in freshly-cut segments whereas resveratrol, piceatannol, and isorhapontigenin (3′-O-methylated piceatannol) started to accumulate 72 h after wounding (FIG. 7B, FIG. 18A-18C, and Table 8). A miniscule amount of a hexoside of piceatannol was also tentatively identified (FIGS. 19A-19B). Overall, mechanically-wounded wild sugarcane produced piceatannol and its B-ring O-methylated derivative isorhapontigenin which constituted a small portion of the stilbene profile (FIG. 7B), while no A-ring O-methylated stilbene could be detected. Next, the understanding of isorhapontigenin biosynthesis was pursued in wild sugarcane. Substantial upregulation of SsSTS gene expression (Sspon.06G0010290-2P) 72 h after wounding was detected (FIG. 20A, FIG. 20C & FIG. 21). Under in vitro condition, recombinant SsSTS generated piceatannol with caffeoyl-CoA as the starter substrate but failed to generate isorhapontigenin when feruloyl-CoA was used (FIGS. 22A-22E). Hence, SsSTS is a functional STS but an OMT is most likely required for 3′-O-methylation of piceatannol to produce isorhapontigenin. Meanwhile, a canonical SsCOMT (Sspon.06g0010980-3C) highly conserved with SbCOMT (>94.0% protein sequence identity) was identified in wild sugarcane (FIG. 14 & FIG. 23). It also harbors the key Asn residues corresponding to Asn128 and Asn323 in SbCOMT (FIG. 23). In fact, recombinant enzyme assays demonstrated that SsCOMT and SbCOMT showed the same catalytic regioselectivity. Accordingly, isorhapontigenin, ferulic acid, and sinapic acid were generated when SsCOMT was incubated with piceatannol, caffeic acid, and 5-hydroxyferulic acid respectively (FIG. 7C & FIGS. 24A-24F). However, only small amounts of pinostilbene were generated when resveratrol was used as substrate (FIGS. 24G-24I). In addition, SsCOMT shares similar substrate-binding properties with SbCOMT (Table 9). Gene expression further revealed that SsCOMT is constitutively expressed in the wild sugarcane segments despite its downregulation after wounding (FIG. 20B & FIG. 20D). Taken together, SsCOMT is an important candidate for isorhapontigenin production through 3′-O-methylation of piceatannol in the B-ring.

### Discussion

O-Methylated stilbenes are well-acclaimed for their health-promoting benefits and exceptional bioavailability³⁵⁻⁴⁸. Sorghum is a major staple crop and wild sugarcane is a genetic resource for sugarcane breeding. In this study, the potential of these Sacharinae grasses as biofactories for O-methylated stilbenes as well as the OMTs for regioselective stilbene O-methylations were revealed. The findings presented herein also provide insights into bioengineering of specific O-methylated stilbenes via molecular breeding and transgenic approaches, representing a unique opportunity to improve dietary intake of these nutraceuticals which are scarcely present in natural food sources. Recent breakthroughs in Sorghum biotechnology, including stable transformation and

CRISPR/Cas9-mediated genome editing as well as its metabolic versatility, have warranted Sorghum as an emerging model for investigation and manipulation of specialized metabolism⁴⁹⁻⁵¹. Here, complete depletion of O-methylated stilbenes in infected Sorghum SbSOMT CRISPR-Cas9 mutants (FIGS. 3A-3E) firmly established SbSOMT as the primary SOMT for pathogen-inducible pterostilbene biosynthesis. Concomitantly, molecular, biochemical, and structural characterizations were integrated to elucidate the mechanistic details of SbSOMT-stilbene reactions. The first crystal structure of a bona fide stilbene O-methyltransferase, SbSOMT is reported herein. The enzyme utilizes the His282-Asp283-Glu310-Glu342 catalytic residues to generate 3,5-bis-O-methylated stilbenes (FIG. 2A, FIGS. 4B-4C, FIGS. 5B-5F & FIGS. 16A-16C). The same catalytic residues are found in canonical COMTs, indicating the highly conserved O-methylation catalysis irrespective of phenolic substrates involved13, 21, 22, 52, 53. It was demonstrated that Glu310 positions the 5-OH group, instead of the more hydrophobic 3-OCH3 group, in close proximity to His282 for the second O-methylation (FIGS. 5C-5D & FIG. 16B). As Glu310 is highly conserved in most OMTs (with some harboring an Asp), this feature may represent a common mechanism for mediating successive O-methylations of structurally different phenolic substrates.

Meanwhile, combinatorial analyses presented herein rationalized the regioselectivities of SbSOMT and canonical COMTs underlain by specific substrate binding modes instead of alterations in catalysis or substrate affinity (FIG. 2A, FIGS. 4B-4C, FIGS. 5B-5F, FIGS. 6A-6D, FIGS. 7A-7C, Table 1, Table 2, FIG. 17 & Table 7). The hydrophilic Asn128/Asn323 residues in SbCOMT, which are highly conserved in COMTs from diverse plant lineages, orchestrate productive coordination with their native substrates, i.e. hydroxycinnamic acids and their analogs, via hydrogen bonding^{21, 22}. The same binding mechanism is apparently crucial for attaining productive and energetically favorable orientation of piceatannol inside the binding pocket of Saccharinae COMTs including SbCOMTAsn128/Asn323 and SsCOMTAsn130/Asn323 for B-ring O-methylation (FIG. 5E & FIGS. 24A-24B). In marked comparison, the recruitment of more hydrophobic residues in SbSOMT like Ile144 and Phe337 during its emergence in the Sorghum genus would considerably favor hydrophobic interactions with the C4 and C3′ of resveratrol (and piceatannol) and pinostilbene (and 3′-hydroxypinostilbene), hence favoring A-ring O-methylation (FIGS. 4B-4C, FIG. 5E & FIGS. 6C-6D).

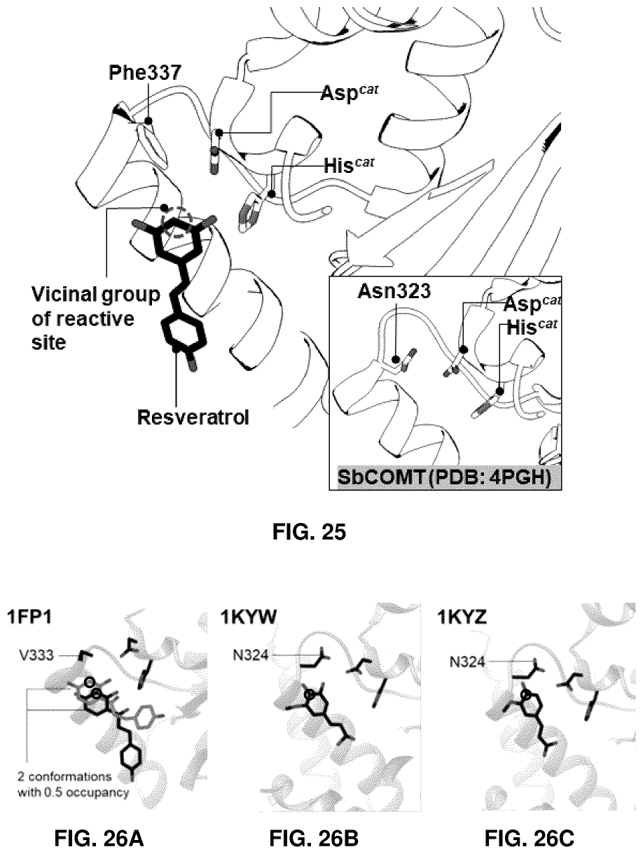

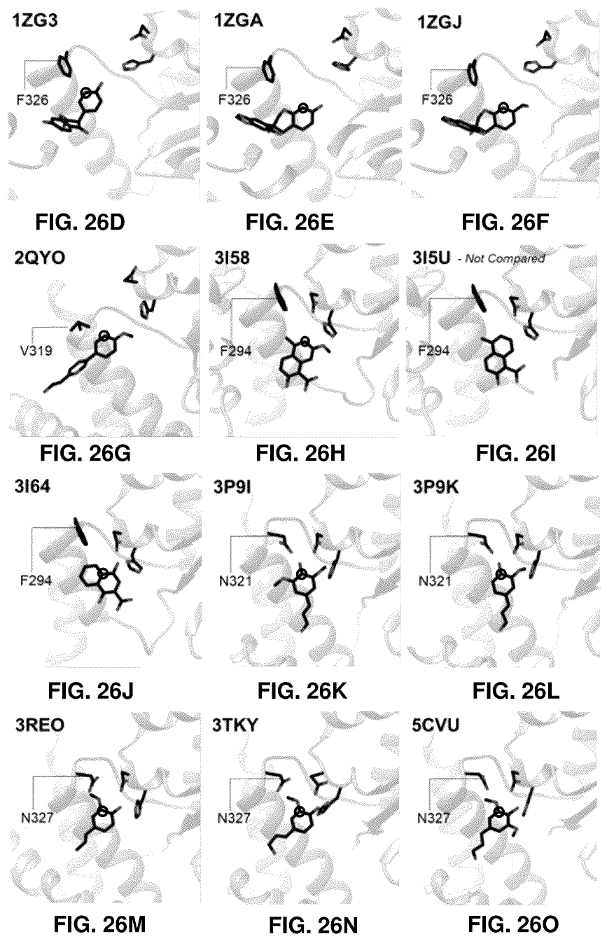

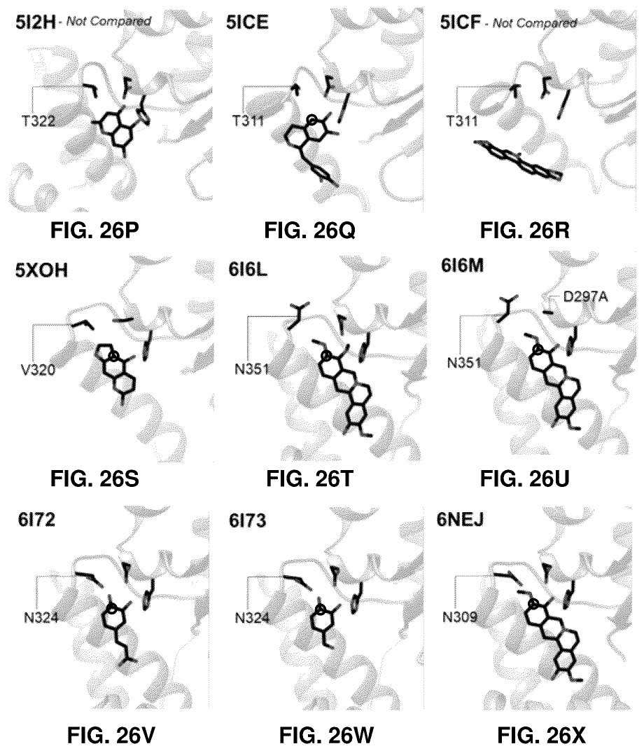

Thorough mining of the Protein Data Bank (PDB) retrieved a diverse panel of 24 OMT-ligand complexes (from 13 OMTs with ≥30% protein identity to SbCOMT/SbSOMT), supporting a general occurrence of compatible polarity pairing between the amino acid residue equivalent to SbCOMTAsn323/SbSOMTPhe337 in OMTs and their substrates (Table 10). It was reasoned that upon conformational transitions, this residue and the catalytic residues are brought into close proximity, leading to compatible polarity pairing between 12 (out of 13) OMTs and their respective substrate (FIG. 25 & FIGS. 26A-26X). For example, six OMTs harboring an Asn residue at this position is paired to a polar-OH or -OCH3 moiety of the ligand (Table 10 & FIGS. 26A-26X)^{43, 44, 46, 54-57}. For the others, the equivalent residue is a more hydrophobic residue (Thr, Val or Phe) which is paired to a non-polar group such as a methyl group or an aromatic ring of the ligand (Table 10 & FIGS. 26A-26X). Consistently, two previously characterized SOMTs, VvROMTPhe318 and SbOMT3-Ile336, harbor a hydrophobic residue at this equivalent position^{28, 29}. Meanwhile, naringenin OMTs, which harbor a hydrophobic Leu residue (OsNOMTLeu335; SbNOMTLeu324; ZmNOMTLeu325) paired to the aromatic carbon (C6), catalyze 7-O-methylation (A-ring) of naringenin to generate sakuranetin, but do not utilize hydroxycinnamic acid substrates⁵⁸. Collectively, the polarity of the amino acid residue in OMTs equivalent to SbCOMTAsn323/SbSOMTPhe337 may govern substrate selectivity through a compatible polarity pairing with the functional group vicinal to the methyl-accepting —OH group in a substrate (FIG. 25 & FIGS. 26A-26X). Consequently, replacement of Asn323 by a hydrophobic residue represents a key molecular event towards the functional divergence of regioselective OMT activities from canonical COMTs in plants. On the other hand, polarity pairing between ligand and SbCOMTAsn128 is only observed in some OMT-ligand complexes, as the equivalent residues in OMTs are diversified (Table 10). Overall, the findings presented herein underpin subsequent bio-engineering of OMTs, either by directed evolution or targeted site-directed mutagenesis, for production of regioselective O-methylated phenolic nutraceuticals/pharmaceuticals in vitro or in planta.

Sorghum and wild sugarcane are closely-related species but resveratrol is their only common stilbene detected by experimental conditions used herein. In addition to resveratrol, Sorghum produces pinostilbene and pterostilbene upon fungal infection while wild sugarcane accumulates resveratrol, piceatannol, and isorhapontigenin following mechanical wounding (FIGS. 1A-1F & FIGS. 7A-7C). The lack of B-ring O-methylated stilbenes (pinostilbene and pterostilbene) in wounded wild sugarcane stems primarily from the absence of SbSOMT orthologs, which are unique to Sorghum spp. On the other hand, the presence of isorhapontigenin (B-ring O-methylated) is resulting from canonical SsCOMT activities although genetic evidence is required for confirmation. Meanwhile, the underlying causes for the exclusivity of piceatannol (3′-hydroxylated) and related stilbenes in Saccharum spp. within the Saccharinae subtribe remain elusive. Elucidation of the piceatannol biosynthetic pathway, in combination with the aforementioned bio-engineering of OMTs for targeted stilbene O-methylation, would further unleash the potential of these Saccharinae crops for stilbene biofortification.

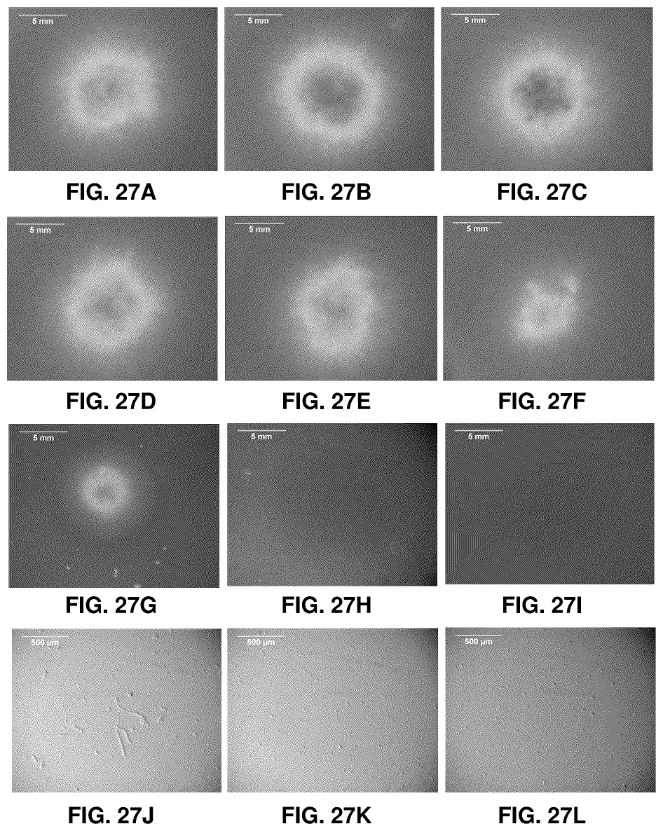

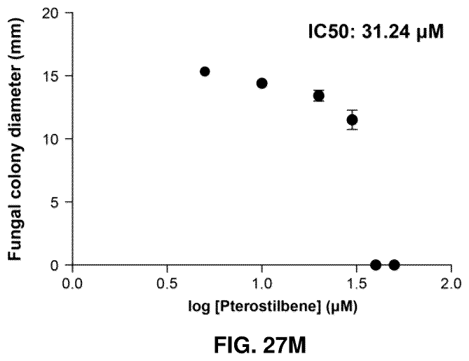

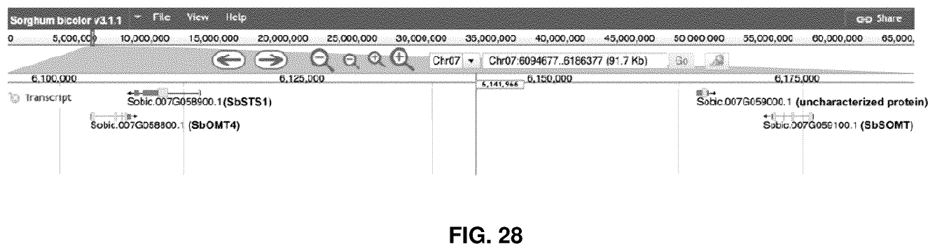

Apparently, Sorghum-specific SOMTs was originated from the canonical and ubiquitous COMTs in Poaceae (FIG. 2D). The COMT-to-SOMT evolution fits well into the generally-accepted Yeas-Jensen model which suggests that a promiscuous enzyme with poor activities towards specific substrates often serves as the basic scaffold undergoing sequence divergence (e.g. the aforementioned replacement of SbCOMTAsn323 by SbSOMTPhe337) to optimize catalytic efficiency59, 60. Further nonsynonymous mutations within an ancestral Sorghum COMT allowed neofunctionalization with acquisition of efficient resveratrol 3,5-bis-O-methylation activities in SbSOMT (FIG. 2A). Such molecular evolution might have been driven by the superior potency of pterostilbene over resveratrol and pinostilbene as a broad-spectrum phytoalexin (FIG. 27A-27M)^{6, 61}. Both SbSOMT and OsNOMT have lost the ability to O-methylate hydroxycinnamic acids (FIG. 2B), which are key in planta substrates of COMTs^{21, 22}. Further investigations will shed new light on whether compromised substrate recognition pattern is necessary, and to what extent, to achieve the highly efficient SOMT activities in SbSOMT. Since both SbSOMT and SbCOMT utilize the same catalytic residues and mechanism for O-methylation, evolution of pterostilbene biosynthesis primarily hinged on optimizing the stilbene binding mode with productive orientations. Furthermore, the recruitment of SbSTS1 from a chalcone synthase scaffold for resveratrol biosynthesis would logically predate that of SbSOMT from SbCOMT5. An increasing number of specialized metabolic pathways are considered to have evolved in such order as gene clusters⁶²⁻⁶⁴. Correspondingly, physically-linked SbSTS1 (Sb07g004700), SbOMT4 (Sb07g004690), and SbSOMT (Sb07g004710) (FIG. 28) were identified as constituents of a gene cluster for specialized metabolism (Cluster 408) in the Sorghum genome⁶³⁻⁶⁵.

TABLE 1
Kinetic parameters of SbSOMT and SbCOMT
with pinostilbene as a substrate.

| | SbCOMT | SbSOMT |
|---|---|---|
| Kₘ (μM) | 3.14 ± 1.40 | 4.62 ± 0.46 |
| k_{cat} (min⁻¹) | 0.14 ± 0.01 | 10.30 ± 0.20 |
| k_{cat}/Kₘ (min⁻¹ μM⁻¹) | 0.045 | 2.229 |
| Specific activity (nmol | 3.46 ± 0.27 | 290.04 ± 5.64 |
| min⁻¹ mg⁻¹) | | |

Values refer to means ± SD (n = 3).

TABLE 2
Dissociation constants of SbSOMT, SbCOMT with stilbenes.

K_{d} (μM)

| | SbCOMT | SbSOMT |
|---|---|---|
| Resveratrol | 1.55 ± 0.13 | 4.69 ± 0.54 |
| Pinostilbene | 1.79 ± 0.09 | 2.86 ± 0.33 |
| Pterostilbene | 3.99 ± 0.24 | 11.20 ± 2.23 |
| Piceatannol | 1.26 ± 0.06 | 37.50 ± 4.33 |
| SAM | 13.00 ± 0.83 | 90.00 ± 9.59 |

Results are expressed as means of K_{d} ± standard error derived from curve fitting.

TABLE 3
High-performance liquid chromatography-quadrupole time-of-flight
MS (HPLC-QTOF-MS) analysis of flavone aglycones in control or
Colletotrichum sublineola-infected mesocotyls of resistant
(SC748-5) and susceptible (BTx623) sorghum genotypes.

| | BTx623 | | SC748-5 | |
|---|---|---|---|---|
| Time (h) | Control | Infected | Control | Infected |

Apigenin [peak area (cps × 10⁶) g⁻¹ FW]

| | | | | |
|---|---|---|---|---|
| 24 | 4.08 ± 0.19 | 5.48 ± 4.58 | 3.95 ± 0.57 | 4.10 ± 0.30 |
| 48 | 7.18 ± 2.34 | 12.41 ± 1.67 | 8.06 ± 0.83 | 52.27 ± 12.73 |
| 72 | 12.25 ± 1.93 | 105.55 ± 31.98 | 12.24 ± 2.55 | 33.61 ± 9.98 |
| 96 | 13.11 ± 1.04 | 272.56 ± 100.65 | 7.42 ± 1.71 | 20.59 ± 3.92 |

Luteolin [peak area (cps × 10⁶) g⁻¹ FW]

| | | | | |
|---|---|---|---|---|
| 24 | 1.20 ± 0.25 | 0.11 ± 0.05 | 2.38 ± 0.57 | 2.57 ± 0.45 |
| 48 | 1.16 ± 0.11 | 1.66 ± 0.43 | 3.37 ± 1.35 | 158.69 ± 29.51 |
| 72 | 1.62 ± 0.09 | 21.66 ± 10.97 | 4.94 ± 1.60 | 512.03 ± 65.46 |
| 96 | 5.35 ± 0.82 | 65.99 ± 32.20 | 7.03 ± 2.30 | 497.52 ± 135.58 |

Chrysoeriol [peak area (cps × 10⁶) g⁻¹ FW]

| | | | | |
|---|---|---|---|---|
| 24 | 3.19 ± 2.60 | 4.66 ± 0.87 | 4.75 ± 1.15 | 8.88 ± 0.86 |
| 48 | 6.18 ± 0.27 | 8.02 ± 0.64 | 9.54 ± 1.11 | 108.23 ± 14.69 |
| 72 | 10.26 ± 1.15 | 28.49 ± 5.22 | 13.31 ± 1.97 | 308.20 ± 24.77 |
| 96 | 12.91 ± 1.07 | 75.03 ± 21.31 | 14.01 ± 4.62 | 364.55 ± 100.89 |

Tricin [peak area (cps × 10⁷) g⁻¹ FW]

| | | | | |
|---|---|---|---|---|
| 24 | 2.58 ± 0.27 | 1.59 ± 1.17 | 41.14 ± 9.33 | 35.36 ± 2.76 |
| 48 | 2.75 ± 0.55 | 2.62 ± 0.20 | 59.66 ± 10.28 | 75.39 ± 16.11 |
| 72 | 3.80 ± 1.03 | 4.52 ± 0.53 | 88.92 ± 6.14 | 242.87 ± 31.12 |
| 96 | 4.58 ± 0.04 | 9.76 ± 1.42 | 73.18 ± 24.74 | 396.46 ± 22.25 |

Compound annotation was achieved by comparing the accurate mass and fragmentation pattern of individual peaks with those from authentic standards. Apigenin-d₅ was used as an internal standard for quantitation. Values refer to means ± SD (n = 3).
cps: count per second, FW: fresh weight.

TABLE 4
High-performance liquid chromatography-quadrupole time-of-flight MS
(HPLC-QTOF-MS) analysis of 3-deoxyanthocyanidin aglycones in mock
or Colletotrichum sublineola-infected mesocotyls of resistant
(SC748-5) and susceptible (BTx623) sorghum genotypes.

| | BTx623 | | SC748-5 | |
|---|---|---|---|---|
| Time (h) | Control | Infected | Control | Infected |

Apigeninidin [peak area (cps × 10⁸) g⁻¹ FW]

| | | | | |
|---|---|---|---|---|
| 48 | n.d. | 1.11 ± 0.25 | n.d. | 4.69 ± 1.65 |
| 72 | n.d. | 10.52 ± 5.66 | n.d. | 10.93 ± 1.72 |
| 96 | n.d. | 19.37 ± 2.09 | n.d. | 9.49 ± 1.24 |

Luteolinidin [peak area (cps × 10⁶) g⁻¹ FW]

| | | | | |
|---|---|---|---|---|
| 48 | n.d. | 2.44 ± 0.65 | n.d. | 822.19 ± 271.66 |
| 72 | n.d. | 153.69 ± 94.92 | n.d. | 2735.93 ± 339.38 |
| 96 | n.d. | 246.27 ± 45.66 | n.d. | 2403.59 ± 761.50 |

Diosmetinidin [peak area (cps × 10⁶) g⁻¹ FW]

| | | | | |
|---|---|---|---|---|
| 48 | n.d. | 0.97 ± 0.12 | n.d. | 167.38 ± 78.93 |
| 72 | n.d. | 21.13 ± 12.19 | n.d. | 803.91 ± 213.56 |
| 96 | n.d. | 46.81 ± 10.43 | n.d. | 1352.29 ± 185.15 |

Compound annotation was achieved by comparing the accurate mass and fragmentation pattern of individual peaks with those from authentic standards. Apigenin-d₅ was used as an internal standard for quantitation. Values refer to means ± SD (n = 3).
cps: counts per second, FW: fresh weight, n.d.: not detected.

TABLE 5
High-performance liquid chromatography-quadrupole time-of-flight MS
(HPLC-QTOF-MS) analysis of stilbenes in mock or
Colletotrichum sublineola-infected mesocotyls of resistant (SC748-5)
and susceptible (BTx623) sorghum genotypes.

| | BTx623 | | SC748-5 | |
|---|---|---|---|---|
| Time (h) | Control | Infected | Control | Infected |

Resveratrol (μg g⁻¹ FW)

| | | | | |
|---|---|---|---|---|
| 72 | n.d. | 0.03 ± 0.01 | n.d. | 0.31 ± 0.00 |
| 96 | n.d | 0.47 ± 0.23 | n.d. | 0.91 ± 0.19 |

Piceid (μg g⁻¹ FW)

| | | | | |
|---|---|---|---|---|
| 72 | n.d. | 2.63 ± 0.23 | n.d. | 7.99 ± 0.46 |
| 96 | n.d. | 6.27 ± 1.27 | n.d. | 19.60 ± 3.24 |

Pinostilbene (μg g⁻¹ FW)

| | | | | |
|---|---|---|---|---|
| 72 | n.d. | 0.05 ± 0.00 | n.d. | 0.22 ± 0.00 |
| 96 | n.d. | 0.07 ± 0.02 | n.d. | 0.57 ± 0.14 |

Pterostilbene (μg g⁻¹ FW)

| | | | | |
|---|---|---|---|---|
| 48 | n.d. | n.d. | n.d. | 0.03 ± 0.02 |
| 72 | n.d. | 0.16 ± 0.06 | n.d. | 1.56 ± 0.13 |
| 96 | n.d. | 0.61 ± 0.23 | n.d. | 5.66 ± 1.89 |

Compound annotation was achieved by comparing the accurate mass and fragmentation pattern of individual peaks with those from authentic standards. Apigenin-d₅ was used as an internal standard for quantitation. Values refer to means ± SD (n = 3).
FW: fresh weight, n.d.: not detected.

TABLE 6
High-performance liquid chromatography-quadrupole time-
of-flight MS (HPLC-QTOF-MS) analysis of stilbenes in mock
or Colletotrichum sublineola-infected mesocotyls
of wild-type Tx430 and sbsomt mutants.

| | | | | |
|---|---|---|---|---|
| Time (h) | Tx430 | sbsomt-a | sbsomt-b1 | sbsomt-b2 |

Resveratrol (μg g⁻¹ FW)

| | | | | |
|---|---|---|---|---|
| 72 | 0.09 ± 0.12 | 0.09 ± 0.02 | 0.10 ± 0.03 | 0.11 ± 0.04 |
| 96 | 0.18 ± 0.03 | 0.31 ± 0.12 | 0.49 ± 0.07 | 0.53 ± 0.03 |

Piceid (μg g⁻¹ FW)

| | | | | |
|---|---|---|---|---|
| 72 | 1.60 ± 0.25 | 2.60 ± 1.20 | 1.78 ± 1.06 | 1.97 ± 1.34 |
| 96 | 5.74 ± 0.70 | 8.03 ± 2.33 | 18.14 ± 2.32 | 17.14 ± 5.45 |

Pinostilbene (μg g⁻¹ FW)

| | | | | |
|---|---|---|---|---|
| 72 | 0.03 ± 0.00 | n.d. | n.d. | n.d. |
| 96 | 0.09 ± 0.02 | n.d. | n.d. | n.d. |

Pterostilbene (μg g⁻¹ FW)

| | | | | |
|---|---|---|---|---|
| 72 | 0.13 ± 0.02 | n.d. | n.d. | n.d. |
| 96 | 1.84 ± 0.32 | n.d. | n.d. | n.d. |

Compound annotation was achieved by comparing the accurate mass and fragmentation pattern of individual peaks with those from authentic standards. Apigenin-d₅ was used as an internal standard for quantitation. Values refer to means ± SD (n = 3).
FW: fresh weight, n.d.: not detected.

TABLE 7
Dissociation constant (K_{d}) of SbSOMT mutants with stilbenes

| Stilbene | SbSOMT mutant | Stoichiometry, N | K_{d} (μM) |
|---|---|---|---|
| Resveratrol | I144N | 0.6 | 151.6 ± 31.1 |
| | D283A | 0.58 ± 0.01 | 1.31 ± 0.13 |
| | E310A | 0.36 ± 0.04 | 18.5 ± 2.58 |
| | F337N | — | No binding |
| | E342A | 0.75 ± 0.01 | 11.1 ± 0.75 |
| | I144N/F337N | — | No binding |
| | H282N/D283A | 0.61 ± 0.17 | 32.90 ± 5.93 |
| Pinostilbene | E310A | 0.59 ± 0.01 | 3.91 ± 0.33 |
| Pterostilbene | E310A | 0.40 ± 0.08 | 6.04 ± 2.08 |

Results are expressed as means of K_{d} ± SE derived from curve fitting.
N of I144N was fixed at 0.6 (average to reported Ns in this table) to estimate the K_{d}.

TABLE 8
High-performance liquid chromatography-quadrupole
time-of-flight MS (HPLC-QTOF-MS) analysis of stilbenes
in mechanically-wounded stalks wild sugarcane.

| | Wounded wild sugarcane | | |
|---|---|---|---|
| | Resveratrol | Piceatannol | Isorhapontigenin |
| Time (h) | (μg g⁻¹ FW) | (μg g⁻¹ FW) | (μg g⁻¹ FW) |
| 0 | n.d. | n.d. | n.d. |
| 24 | n.d. | n.d. | n.d. |
| 72 | 2.55 ± 1.55 | 9.37 ± 5.98 | 1.03 ± 0.87 |
| 120 | 7.87 ± 3.03 | 33.79 ± 4.50 | 2.18 ± 1.77 |

Compound annotation was achieved by comparing the accurate mass and fragmentation pattern of individual peaks with those from authentic standards. Apigenin-d₅ was used as an internal standard for quantitation. Values refer to means ± SD (n = 3).
FW: fresh weight, n.d.: not detected.

TABLE 9
Dissociation constants of SsCOMT with stilbenes.

| | K_{d} (μM) |
|---|---|
| | SsCOMT |
| Resveratrol | 2.78 ± 0.33 |
| Pinostilbene | 5.57 ± 0.77 |
| Pterostilbene | 9.93 ± 4.04 |
| Piceatannol | 25.50 ± 14.20 |
| SAM | 25.00 ± 12.90 |

Results are expressed as means of K_{d} ± standard error derived from curve fitting.

TABLE 10
List of OMT-ligand complexes deposited to PDB and the equivalent residues
of SbCOMT^{Asn128}/SbSOMT^{Ile144} and SbCOMT^{Asn323}/SbSOMT^{Phe337}.

| | | Residue at equivalent | | | | | |
|---|---|---|---|---|---|---|---|
| | | positions | | | | | |
| | | SbCOMT | SbCOMT | | Vicinal | | |
| | | Asn128 | Asn323 | | functional | SEQ | |
| PDB | | SbSOMT | SbSOMT | Ligand | group^{#} and | ID | |
| ID | UNIPROT | Ile144 | Phe337 | identity | polarity | NO | Ref. |
| 1FP1 | P93324 | Leu139 | Val333 | Substrate | —H and non-polar | 82 | [43] |
| 1KYW | P28002 | Asn131 | Asn324 | Substrate | —OH and polar | 83 | [44] |
| 1KYZ | P28002 | Asn131 | Asn324 | Product | —OH and polar | | |
| 1ZG3 | Q29U70 | Ala126 | Phe326 | Substrate | —H and non-polar | 84 | [54] |
| 1ZGA | Q29U70 | Ala126 | Phe326 | Substrate | —H and non-polar | | |
| 1ZGJ | Q29U70 | Ala126 | Phe326 | Product | —H and non-polar | | |
| 2QYO | Q06YR3 | Val123 | Val319 | Canonical | —H and non-polar | 85 | NA |
| | | | | product | | | |
| 3I58 | Q84HC8 | Asp107 | Phe294 | Product | —H and non-polar | 86 | [55] |
| 3I5U | Q84HC8 | Asp107 | Phe294 | Substrate | Not compared | | |
| | | | | analogue (non- | | | |
| | | | | reactive) | | | |
| 3I64 | Q84HC8 | Asp107 | Phe294 | Substrate | Fused benzene ring | | |
| | | | | analogue | and non-polar | | |
| | | | | (reactive) | | | |
| 3P9I | Q9ZTU2 | Asn128 | Asn321 | Product | —OH and polar | 87 | [22] |
| 3P9K | Q9ZTU2 | Asn128 | Asn321 | Product | —OH and polar | | |
| 3REO | O04385 | Ala134 | Asn327 | Substrate | —OMe and polar | 88 | |
| 3TKY | O04385 | Ala134 | Asn327 | Substrate | —OMe and polar | | |
| 5CVU | O04385 | Ala134 | Asn327 | Substrate | —OMe and polar | | |
| 5I2H | D5STZ7 | Ala110 | Thr322 | Non-productive | Not compared | 89 | |
| | | | | conformation | | | |
| 5ICE | Q5C9L7 | Ile114 | Thr311 | Substrate | —H and non-polar | 90 | |
| 5ICF | Q5C9L7 | Ile114 | Thr311 | Non-reactive | Not compared | | |
| | | | | inhibitor | | | |
| 5XOH | A0A166U5H3 | His126 | Val320 | Substrate | Fused furan ring and | 91 | |
| | | | | | non-polar | | |
| 6I6L | I3V6A7 | Thr157 | Asn351 | Product | —OMe and polar | 92 | |
| 6I6M | I3V6A7 | Thr157 | Asn351 | Substrate | —OMe and polar | | |
| 6I72 | Q9M602 | Asn131 | Asn324 | Canonical | —OH and polar | 93 | |
| | | | | substrate | | | |

^{#}The vicinal functional group refers to the functional group vicinal to the reactive site positioned between catalytic dyads and SbCOMTAsn323/SbSOMTPhe337 equivalent position as depicted in FIG. 25 & FIGS. 26A-26X.
NA, Not available (work to be published).

TABLE 11
Primers.

| | Sequence (5′ to 3′) | |
|---|---|---|
| | (restriction sites | |
| Primer name | added are in italic) | Purposes |
| SbEIF4a-qRT-F | CAACTTTGTCACCCGCGATGA (SEQ | qRT-PCR experiments for SbEIF4α |
| | ID NO: 36) | |
| SbEIF4a-qRT-R | TCCAGAAACCTTAGCAGCCCA (SEQ | |
| | ID NO: 37) | |
| SbSTS-qRT-F | TGCTACGGTGTTGGCCATT (SEQ ID | qRT-PCR experiments for SbSTS1 |
| | NO: 38) | |
| SbSTS-qRT-R | ATCGACTTGTGGCATATCCTCTT | |
| | (SEQ ID NO: 39) | |
| SbOMT4-qRT-F | CCTAAAGTGGATTCTTCACGATTG | qRT-PCR experiments for SbOMT4 |
| | (SEQ ID NO: 40) | |
| SbOMT4-qRT-R | GCTAGTTGCGATGTTAGTGTTTC | |
| | (SEQ ID NO: 41) | |
| SbSOMT-qRT-F | AGGTGGCAGCATGTTCGATAA (SEQ | qRT-PCR experiments for SbSOMT |
| | ID NO: 42) | |
| SbSOMT-qRT-R | ACGATCACCTTGCCTCTCAC (SEQ ID | |
| | NO: 43) | |
| SsGADPH-qRT-F | TTGGTTTCCACTGACTTCGTT (SEQ ID | qRT-PCR experiments for SsGADPH |
| | NO: 44) | |
| SsGADPH-qRT-R | CTGTAGCCCCACTCGTTGT (SEQ ID | |
| | NO: 45) | |
| SsSTS-qRT-F | AGAGCGAACACCTTACCGAC (SEQ ID | qRT-PCR experiments for SsSTS |
| | NO: 46) | |
| SsSTS-qRT-R | GCCGAGTACGAGCTCATGTT (SEQ ID | |
| | NO: 47) | |
| SsCOMT-qRT-F | GAGGACAAGGACGGCAAGTA (SEQ | qRT-PCR experiments for SsCOMT |
| | ID NO: 48) | |
| SsCOMT-qRT-R | ACCGCGTCCTTGAGGTAGTA (SEQ ID | |
| | NO: 49) | |
| SbOMT4-RE-F | CGCGGATCCATGGGCAGCTATACTAC | Cloning of SbOMT4 in pET23a(+) |
| | CAG (SEQ ID NO: 50) | vector for recombinant protein |
| SbOMT4-RE-R | CCCAAGCTTCTTTGTGAATTCAAGGG | expression in E. coli |
| | CCC (SEQ ID NO: 51) | |
| SbSOMT-HF-F | TGAGAACCTGTACTTCCAAGGCAGC | HiFi Assembly of SbSOMT (2-377) in |
| | TACGACAGCAGCAGTAG (SEQ ID | pET-N-His-TEV vector for recombinant |
| | NO: 52) | |
| SbSOMT-HF-R | AGCCGGATCTCACTCGAGTTACTTTG | protein expression in E. coli |
| | TGAACTCAAGGGCCCAGAC (SEQ ID | |
| | NO: 53) | |
| SbCOMT-HF-F | TGAGAACCTGTACTTCCAAGGGTCG | HiFi Assembly of SbCOMT (2-362) in |
| | ACGGCGGAGGAC (SEQ ID NO: 54) | \|PET-N-His-TEV vector for recombinant |
| SbCOMT-HF-R | AGCCGGATCTCACTCGAGTTACTTGA | protein expression in E. coli |
| | TGAACTCGATGGCCCAG (SEQ ID | |
| | NO: 55) | |
| SsSTS-Gib-F | CTTCTGCAGGAATTCGATATCATGAC | Gibson Assembly of SsSTS in pET-N- |
| | TGGGAAGGTAACATTGGGG (SEQ ID | His-TEV vector for recombinant protein |
| | NO: 56) | |
| SsSTS-Gib-R | GAGAGATCTGTCGACGATATCCTAC | expression in E. coli |
| | ACTGTGATGATGGGAACGC (SEQ ID | |
| | NO: 57) | |
| SsCOMT-Gib-F | CTTCTGCAGGAATTCGATATCATGGG | Gibson Assembly of SsCOMT in pET- |
| | CTCGACCGCC (SEQ ID NO: 58) | N-His-TEV vector for recombinant |
| SsCOMT-Gib-R | GAGAGATCTGTCGACGATATCTTACT | protein expression in E. coli |
| | TGATGAACTCGATGGCCCAG (SEQ ID | |
| | NO: 59) | |
| SbSOMT-1144N-F | GTGCTTCCGCTTGGGATGATGAACCT | Generation of SbSOMT 1144N and |
| | AAACAAGACATTCCTGGACAGC (SEQ | |
| | ID NO: 60) | |
| SbSOMT-1144N-R | CATCATCCCAAGCGGAAGCAC (SEQ | I144N/F337N mutant proteins |
| | ID NO: 61) | |
| SbSOMT-F337N-F | CCTCACCATGCTGGTCACGAACGGC | Generation of SbSOMT F337N and |
| | AGTGGTAAAGAGAGGACACA (SEQ | |
| | ID NO: 62) | |
| SbSOMT-F337N-R | CGTGACCAGCATGGTGAGG (SEQ ID | I144N/F337N mutant proteins |
| | NO: 63) | |
| SbSOMT-H282A-F | TTCTGCTCAAGTGGATTCTTGCTGAT | Generation of SbSOMT H282A mutant |
| | TGGGACGACAAGGCGTG (SEQ ID | |
| | NO: 64) | |
| SbSOMT-H282A-R | AAGAATCCACTTGAGCAGAACTGCA | protein |
| | (SEQ ID NO: 65) | |
| SbSOMT-H282N-F | GATTGGGACGACAAGGCGTG (SEQ | Generation of SbSOMT H282N mutant |
| | ID NO: 66) | protein |
| SbSOMT-H282N-R | GTTAAGAATCCACTTGAGCAGAAC | Generation of SbSOMT H282N and |
| | (SEQ ID NO: 67) | H282N/D283A mutant proteins |
| SbSOMT-D283A-F | GCGTGGGACGACAAGG (SEQ ID | Generation of SbSOMT D283A and |
| | NO: 68) | H282N/D283A mutant proteins |
| SbSOMT-D283A-R | ATGAAGAATCCACTTGAGCAGAAC | Generation of SbSOMT D283A mutant |
| | (SEQ ID NO: 69) | protein |
| SbSOMT-E310A-F | GCGTACGTTGTTCCGGATG (SEQ ID | Generation of SbSOMT E310A mutant |
| | NO: 70) | |
| SbSOMT-E310A-R | CAGAACGATCACCTTGCCTC (SEQ ID | protein |
| | NO: 71) | |
| SbSOMT-E342A-F | GCGAGGACACAGAGGGAGTTC (SEQ | Generation of SbSOMT E342A mutant |
| | ID NO: 72) | |
| SbSOMT-E342A-R | TTTACCACTGCCAAACGTGAC (SEQ | protein |
| | ID NO: 73) | |
| sbsomt-gPCR-F1 | TGACTCATCAGCACGGAACG (SEQ ID | Genotyping of sbsomt CRISPR/Cas9 |
| | NO: 74) | |
| sbsomt-gPCR-R1 | CACCCTCCTAGTAGCCGCAA (SEQ ID | mutants |
| | NO: 75) | |
| sbsomt-gPCR-F2 | GGCAAAACATTACGGATGCAGT (SEQ | Genotyping of sbsomt CRISPR/Cas9 |
| | ID NO: 76) | |
| sbsomt-gPCR-R2 | TGACTTCACGGAGGCTCATAG (SEQ | mutants |
| | ID NO: 77) | |
| SbSTS-PEAQ-F | ATCGGACCGGTATGACGACTGGGAA | Cloning of SbSTSI in pEAQ-HT vector |
| | GGTAAC (SEQ ID NO: 78) | |
| SbSTS-PEAQ-R | CGATCCTCGAGTCATGCAGCCACTGT | for N. benthamiana expression |
| | GGTGA (SEQ ID NO: 79) | |
| SbSOMT-PEAQ-F | TGCCCAAATTCGCGAATGGGCAGCT | Cloning of SbSOMT in pEAQ-HT |
| | ACGACAGC (SEQ ID NO: 80) | |
| SbSOMT-PEAQ-R | TGGTGATGGTGATGCATATTATTTTT | vector for N. benthamiana expression |
| | CAAATTGAGGATGAGACCACTTTGT | |
| | GAACTCAAGGGCCCAG (SEQ ID | |
| | NO: 81) | |

TABLE 12
Protein and ligand concentrations used for ITC setup.

| | Protein concentration | | Ligand concentration |
|---|---|---|---|
| Protein in cell | (μM) | Ligand in syringe | (μM) |
| SbSOMT, WT | 50 | Resveratrol | 750 |
| | 50 | Pinostilbene | 750 |
| | 25 | Pterostilbene | 500 |
| | 50 | Piceatannol | 1000 |
| | 50 | SAM | 2000 |
| SbCOMT, WT | 50 | Resveratrol | 750 |
| | 50 | Pinostilbene | 750 |
| | 25 | Pterostilbene | 500 |
| | 50 | Piceatannol | 750 |
| | 50 | SAM | 1500 |
| SbSOMT mutants: | | | |
| I144N | 25 | Resveratrol | 1000 |
| D283A | 50 | Resveratrol | 1000 |
| | 50 | Resveratrol | 1000 |
| E310A | 50 | Pinostilbene | 1000 |
| | 25 | Pterostilbene | 500 |
| F337N | 25 | Resveratrol | 1000 |
| E342A | 50 | Resveratrol | 1000 |
| I144N/F337N | 25 | Resveratrol | 1000 |
| H282N/D283A | 25 | Resveratrol | 1000 |
| SsCOMT, WT | 50 | Resveratrol | 750 |
| | 50 | Pinostilbene | 750 |
| | 25 | Pterostilbene | 500 |
| | 50 | Piceatannol | 1000 |
| | 50 | SAM | 2000 |

TABLE 13
X-ray data collection and refinement statistics of disclosed structures.

| | SbSOMT- | | SbSOMT- | | SbSOMT- | | | |
|---|---|---|---|---|---|---|---|---|
| | Resveratrol- | | Pinostilbene- | | Pterostilbene- | | SbSOMT- | |
| | β-NAD | | β-NAD | | β-NAD | | Resveratrol | |
| Data collection | | | | | | | | |
| Wavelength (Å) | 0.9785 | | 0.9784 | | 0.9784 | | 0.9785 | |
| Resolution (Å) ^{a} | 48.42-1.72 | (1.76-1.72) | 19.70-2.10 | (2.14-2.10) | 31.40-2.40 | (2.44-2.40) | 48.91-2.56 | (2.65-2.56) |
| Space group | P 3₁ 2 1 | | P 3₁ 2 1 | | P 3₁ 2 1 | | P 2₁ 2₁ 2₁ | |
| Cell dimensions | | | | | | | | |
| a, b, c (Å) | 96.8, 96.8, 168.0 | | 96.7, 96.7, 166.9 | | 96.5, 96.5, 169.4 | | 97.5, 111.7, 131.1 | |
| α, β, γ (°) | 90, 90, 120 | | 90, 90, 120 | | 90, 90, 120 | | 90, 90, 90 | |
| Total reflections | 1882940 | | 651200 | | 709365 | | 616499 | |
| Unique | 97225 | | 101522 | | 36363 | | 46864 | |
| reflections | | | | | | | | |
| Multiplicity | 19.4 | | 6.4 | | 19.5 | | 13.2 | |
| Completeness | 99.8 | (97.1) | 99.8 | (99.0) | 99.2 | (99.9) | 99.9 | (100.0) |
| (%) ^{a} | | | | | | | | |
| Mean I/sigma (I) ^{a} | 30.2 | (2.9) | 9.2 | (2.0) | 7.5 | (0.8) | 16.3 | (2.4) |
| Wilson B-factor | 22.6 | | 31.43 | | 36.78 | | 52.2 | |
| (Å²) | | | | | | | | |
| R_{merge} (%) ^{a} | 6.0 | (94.1) | 21.8 | (72.4) | 19.9 | (95.5) | 11.8 | (115.6) |
| Rₘₑₐₛ (%) ^{a} | 6.3 | (101.0) | 23.8 | (78.8) | 20.4 | (98.2) | 12.7 | (124.7) |
| Rₚᵢₘ (%) ^{a} | 2.0 | (35.7) | 9.4 | (31.0) | 4.6 | (22.8) | 4.8 | (46.8) |
| CC_{1/2} ^{a} | 1.000 | (0.849) | 0.891 | (0.790) | 0.915 | (0.871) | 0.999 | (0.902) |

^{a} Value relative to the highest resolution shell are given in parentheses.

TABLE 13
X-ray data collection and refinement statistics
of structures reported in this study (continued).

| | SbSOMT- | SbSOMT- | SbSOMT- | |
|---|---|---|---|---|
| | Resveratrol- | Pinostilbene- | Pterostilbene- | SbSOMT- |
| | β-NAD | β-NAD | β-NAD | Resveratrol |
| Refinement | | | | |
| Reflections used in | 97159 | 53133 | 36012 | 46802 |
| refinement | | | | |
| Reflections used for R- | 4899 | 2652 | 1899 | 2368 |
| free | | | | |
| R-work | 0.163 | 0.171 | 0.187 | 0.182 |
| R-free | 0.190 | 0.202 | 0.246 | 0.252 |
| Number of atoms | 6638 | 6216 | 6104 | 11441 |
| Macromolecules | 5841 | 5674 | 5721 | 11286 |
| Ligands/Ions | 229 | 274 | 214 | 92 |
| Solvent | 568 | 268 | 169 | 63 |
| rmsd ^{b} bond lengths | 0.012 | 0.015 | 0.016 | 0.016 |
| (Å) | | | | |
| rmsd ^{b} bond angles (°) | 1.73 | 2.023 | 2.10 | 2.22 |
| Ramachandran plot | | | | |
| Favored (%) | 99.03 | 97.36 | 95.57 | 95.72 |
| Allowed (%) | 0.97 | 2.22 | 3.60 | 3.31 |
| Outlier (%) | 0.00 | 0.42 | 0.83 | 0.97 |
| Sidechain outlier (%) | 0.64 | 2.64 | 1.96 | 4.56 |
| Molprobity Clash score | 4.46 | 8.69 | 7.95 | 8.10 |
| Mean B-value (Å²) | 29.0 | 37.0 | 46.0 | 68.0 |
| Macromolecules | 28.38 | 36.72 | 45.54 | 62.15 |
| Ligands/Ions ^{c} | 53.31 (41.01) | 61.19 | 78.06 | 52.78 |
| Solvent | 36.14 | 39.74 | 43.73 | 41.98 |
| PDB ID code | 7VB8 | 7WAR | 7WAS | 7WAQ |

^{b} rmsd, root mean square deviation.
^{c} The mean B-values (Å²) of ligands and ions are given separately, with value for ion given in parentheses.

TABLE 14
Accession numbers of sequences used in this study.

| Protein | GenBank accession number | SEQ ID NO |
|---|---|---|
| Arabidopsis thaliana AtOMT1 | NP_200227 | 5 |
| Carthamus tinctorius CtCAldOMT | BAG71895 | 6 |
| Catharanthus roseus CrCOMT | Q8W013 | 7 |
| Hordeum vulgare HvCOMT | ABQ58825 | 8 |
| Medicago sativa MsCOMT | P28002 | 9 |
| Medicago sativa MsIOMT | AAC49927 | 10 |
| Oryza sativa OsCAldOMT1 | Q6ZD89 | 11 |
| Oryza sativa OsNOMT | QOIP69 | 12 |
| Panicum virgatum PvCOMT | ADX98508 | 13 |
| Pinus sylvestris PsPMT2 | AQX17823 | 14 |
| Pinus taeda PtAEOMT | Q43096 | 15 |
| Populus tremuloides PtCOMT | AAB61731 | 16 |
| Rosa hybrid RhOOMT | AAM23004 | 17 |
| Solanum lycopersicum SlCOMT | XP_004235028 | 18 |
| Sorghum bicolor SbCOMT | AAO43609 | 2 |
| Sorghum bicolor SbNOMT | XP_002465645 | 19 |
| Sorghum bicolor SbOMT1 | ABP01563 | 20 |
| Sorghum bicolor SbOMT3 | ABP01564 | 21 |
| Sorghum bicolor SbOMT4 | XP_002443937 | 22 |
| Sorghum bicolor SbSOMT | XP_021320201 | 1 |
| Sorghum bicolor SbSTS1 | XP_002445139 | 23 |
| Triticum aestivum TaCOMT1 | AAP23942 | 24 |
| Triticum aestivum TaCOMT2 | Q38J50 | 25 |
| Vitis vinifera VvCOMT | NP_001268100 | 26 |
| Vitis vinifera VvROMT | CAQ76879 | 27 |
| Zea mays ZmCOMT | Q06509 | 28 |
| Zea mays ZmNOMT | XP_020399799 | 29 |

| Gene | Ensembl Plants Identifier | |
|---|---|---|
| Saccharum spontaneum SsSTS | Sspon.06G0010290-2P | 3 |
| Saccharum spontaneum SsCOMT | Sspon.06G0010980-3C | 4 |

| Gene | GenBank accession number | |
|---|---|---|
| Sorghum halepense ShOMT1 | GGDZ01007645 | 30 |
| Sorghum halepense ShSOMT | GGDZ01140964 | 31 |

### REFERENCES

1 Langcake, P., Cornford, C. A. & Pryce, R. J. Identification of pterostilbene as a phytoalexin from Vitis vinifera leaves. Phytochemistry 18, 1025-1027 (1979).
2 Brinker, A. M. & Seigler, D. S. Isolation and identification of piceatannol as a phytoalexin from sugarcane. Phytochemistry 30, 3229-3232 (1991).
3 Chiron, H. et al. Gene induction of stilbene biosynthesis in Scots pine in response to ozone treatment, wounding, and fungal infection. Plant Physiol. 124, 865-872 (2000).
4 Sobolev, V. S., Horn, B. W., Potter, T. L., Deyrup, S. T. & Gloer, J. B. Production of stilbenoids and phenolic acids by the peanut plant at early stages of growth. J. Agric. Food Chem. 54, 3505-3511 (2006).
5 Yu, C. K. et al. A stilbene synthase gene (SbSTS1) is involved in host and nonhost defense responses in Sorghum. Plant Physiol. 138, 393-401 (2005).
6 Sobolev, V. S. et al. Biological activity of peanut (Arachis hypogaea) phytoalexins and selected natural and synthetic Stilbenoids. J. Agric. Food Chem. 59, 1673-1682 (2011).
7 Yu, C. K., Shih, C.-H., Chu, I. K. & Lo, C. Accumulation of trans-piceid in Sorghum seedlings infected with Colletotrichum sublineolum. Phytochemistry 69, 700-706 (2008).
8 Sobolev, V. S., Neff, S. A. & Gloer, J. B. New stilbenoids from peanut (Arachis hypogaea) seeds challenged by an Aspergillus caelatus strain. J. Agric. Food Chem. 57, 62-68 (2009).
9 Jang, M. et al. Cancer chemopreventive activity of resveratrol, a natural product derived from grapes. Science 275, 218-220 (1997).
10 Cosin-Tomas, M. et al. Role of Resveratrol and selenium on oxidative stress and expression of antioxidant and anti-aging genes in immortalized lymphocytes from Alzheimer's disease patients. Nutrients 11 (2019).
11 Chu, X. Y. et al. Isorhapontigenin improves diabetes in mice via regulating the activity and stability of PPARgamma in adipocytes. J. Agric. Food Chem. 68, 3976-3985 (2020).
12 Baerson, S. R. et al. A functional genomics investigation of allelochemical biosynthesis in Sorghum bicolor root hairs. J. Biol. Chem. 283, 3231-3247 (2008).
13 Forster, C. et al. Biosynthesis and antifungal activity of fungus-induced O-methylated flavonoids in maize. Plant Physiol. 188, 167-190 (2022).
14 Fournier-Level, A., Hugueney, P., Verries, C., This, P. & Ageorges, A. Genetic mechanisms underlying the methylation level of anthocyanins in grape (Vitis vinifera L.). BMC Plant Biol. 11, 179 (2011).
15 Du, H. et al. Methylation mediated by an anthocyanin, O-methyltransferase, is involved in purple flower coloration in Paeonia. J. Exp. Bot. 66, 6563-6577 (2015).
16 Fellenberg, C. et al. Tapetum-specific location of a cation-dependent O-methyltransferase in Arabidopsis thaliana. Plant J. 56, 132-145 (2008).
17 Ralph, J., Lapierre, C. & Boerjan, W. Lignin structure and its engineering. Curr Opin. Biotechnol. 56, 240-249 (2019).
18 Saluja, M., Zhu, F., Yu, H., Walia, H. & Sattler, S. E. Loss of COMT activity reduces lateral root formation and alters the response to water limitation in Sorghum brown midrib (bmr) 12 mutant. New Phytol. 229, 2780-2794 (2021).
19 Zhong, R., Morrison III, W. H., Himmelsbach, D. S., Poole II, F. L., & Ye, Z.-H. Essential role of caffeoyl coenzyme A O-methyltransferase in lignin biosynthesis in woody poplar plants. Plant Physiol. 124, 563-578 (2000).
20 Ha, C. M., Rao, X., Saxena, G. & Dixon, R. A. Growth-defense trade-offs and yield loss in plants with engineered cell walls. New Phytol. 231, 60-74 (2021).
21 Green, A. R. et al. Determination of the structure and catalytic mechanism of Sorghum bicolor caffeic acid O-methyltransferase and the structural impact of three brown midrib12 mutations. Plant Physiol. 165, 1440-1456 (2014).
22 Louie, G. V. et al. Structure-function analyses of a caffeic acid O-methyltransferase from perennial ryegrass reveal the molecular basis for substrate preference. Plant Cell 22, 4114-4127 (2010).
23 Walker, A. M. et al. The structure and catalytic mechanism of Sorghum bicolor caffeoyl-CoA O-methyltransferase. Plant Physiol. 172, 78-92 (2016).
24 Ferrer, J.-L., Zubieta, C., Dixon, R. A. & Noel, J. P. Crystal structures of alfalfa caffeoyl coenzyme A 3-O-methyltransferase. Plant Physiol. 137, 1009-1017 (2005).
25 Jung, J.-H. et al. RNA interference suppression of lignin biosynthesis increases fermentable sugar yields for biofuel production from field-grown sugarcane. Plant Biotechnol. J. 11, 709-716 (2013).
26 Barros, J., Temple, S. & Dixon, R. A. Development and commercialization of reduced lignin alfalfa. Curr. Opin. Biotechnol. 56, 48-54 (2019).
27 Paasela, T., Lim, K. J., Pietiainen, M. & Teeri, T. H. The O-methyltransferase PMT2 mediates methylation of pinosylvin in Scots pine. New Phytol. 214, 1537-1550 (2017).
28 Schmidlin, L. et al. A stress-inducible resveratrol O-methyltransferase involved in the biosynthesis of pterostilbene in grapevine. Plant Physiol. 148, 1630-1639 (2008).
29 Rimando, A. M. et al. In planta production of the highly potent resveratrol analogue pterostilbene via stilbene synthase and O-methyltransferase co-expression. Plant Biotechnol. J. 10, 269-283 (2012).
30 Martinez-Márquez, A. et al. Production of highly bioactive resveratrol analogues pterostilbene and piceatannol in metabolically engineered grapevine cell cultures. Plant Biotechnol. J. 14, 1813-1825 (2016).
31 Heo, K. T., Kang, S.-Y. & Hong, Y.-S. De novo biosynthesis of pterostilbene in an Escherichia coli strain using a new resveratrol O-methyltransferase from Arabidopsis. Microb. Cell Fact. 16, 30 (2017).
32 Baur, J. A. & Sinclair, D. A. Therapeutic potential of resveratrol: the in vivo evidence. Nat. Rev. Drug Discov. 5, 493-506 (2006).
33 Bastianetto, S., Dumont, Y., Han, Y. & Quirion, R. Comparative neuroprotective properties of stilbene and catechin analogs: action via a plasma membrane receptor site? CNS Neurosci. Ther. 15, 76-83 (2009).
34 Setoguchi, Y. et al. Absorption and metabolism of piceatannol in rats. J. Agric. Food Chem. 62, 2541-2548 (2014).
35 Dai, Y. et al. Pre-clinical pharmacokinetic and metabolomic analyses of isorhapontigenin, a dietary resveratrol derivative. Front. Pharmacol. 9, 753 (2018).
36 Boue, S. M. et al. Postharvest accumulation of resveratrol and piceatannol in sugarcane with enhanced antioxidant activity. J. Agric. Food Chem. 61, 8412-8419 (2013).
37 Liu, H. et al. Molecular dissection of the pathogen-inducible 3-deoxyanthocyanidin biosynthesis pathway in Sorghum. Plant Cell Physiol. 51, 1173-1185 (2010).
38 Du, Y., Chu, H., Wang, M., Chu, I. K. & Lo, C. Identification of flavone phytoalexins and a pathogen-inducible flavone synthase II gene (SbFNSII) in Sorghum. J. Exp. Bot. 61, 983-994 (2010).
39 Makita, Y. et al. MOROKOSHI: transcriptome database in Sorghum bicolor. Plant Cell Physiol. 56, e6 (2015).
40 Yazawa, T., Kawahigashi, H., Matsumoto, T. & Mizuno, H. Simultaneous transcriptome analysis of Sorghum and Bipolaris sorghicola by using RNA-seq in combination with de novo transcriptome assembly. PLoS One 8, e62460 (2013).
41 Wang, L. et al. Alternative splicing is a Sorghum bicolor defense response to fungal infection. Planta 251, 14 (2019).
42 Cudney, R., Patel, S., Weisgraber, K., Newhouse, Y. & McPherson, A. Screening and optimization strategies for macromolecular crystal growth. Crystallogr. D Biol. Crystallogr. D50, 414-423 (1994).
43 Zubieta, C., He, X.-Z., Dixon, R. A. & Noel, J. P. Structures of two natural product methyltransferases reveal the basis for substrate specificity in plant O-methyltransferases. Nat. Struct. Biol. 8, 271-279 (2001).
44 Zubieta, C., Kota, P., Ferrer, J.-L., Dixon, R. A. & Noel, J. P. Structural basis for the modulation of lignin monomer methylation by caffeic acid/5-hydroxyferulic acid 3/5-O-methyltransferase. Plant Cell 14, 1265-1277 (2002).
45 Robin, A. Y., Giustini, C., Graindorge, M., Matringe, M. & Dumas, R. Crystal structure of norcoclaurine-6-O-methyltransferase, a key rate-limiting step in the synthesis of benzylisoquinoline alkaloids. Plant J. 87, 641-653 (2016).
46 Cabry, M. P. et al. Structure of Papaver somniferum O-methyltransferase 1 reveals initiation of noscapine biosynthesis with implications for plant natural product methylation. ACS Catal. 9, 3840-3848 (2019).
47 Zhang, J. et al. Allele-defined genome of the autopolyploid sugarcane Saccharum spontaneum L. Nat. Genet. 50, 1565-1573 (2018).
48 Yeo, S. C., Ho, P. C. & Lin, H. S. Pharmacokinetics of pterostilbene in Sprague-Dawley rats: the impacts of aqueous solubility, fasting, dose escalation, and dosing route on bioavailability. Mol. Nutr. Food Res. 57, 1015-1025 (2013).
49 Liu, G., Li, J. & Godwin, I. D. Genome editing by CRISPR/Cas9 in Sorghum through biolistic bombardment. Methods Mol. Biol. 1931, 169-183 (2019).
50 Massel, K. et al. Endogenous U6 promoters improve CRISPR/Cas9 editing efficiencies in Sorghum bicolor and show potential for applications in other cereals. Plant Cell Rep. 41, 489-492 (2022).
51 Char, S. N. et al. An Agrobacterium-delivered CRISPR/Cas9 system for targeted mutagenesis in Sorghum. Plant Biotechnol. J. 18, 319-321 (2020).
52 Weng, J.-K., Akiyama, T., Ralph, J. & Chapple, C. Independent recruitment of an O-methyltransferase for syringyl lignin biosynthesis in Selaginella moellendorffii. Plant Cell 23, 2708-2724 (2011).
53 Pluskal, T. et al. The biosynthetic origin of psychoactive kavalactones in kava. Nat. Plants 5, 867-878 (2019).
54 Liu, C.-J. et al. Structural basis for dual functionality of isoflavonoid O-methyltransferases in the evolution of plant defense responses. Plant Cell 18, 3656-3669 (2006).
55 Cooke, H. A., Guenther, E. L., Luo, Y., Shen, B. & Bruner, S. D. Molecular basis of substrate promiscuity for the SAM-dependent O-methyltransferase NcsB1, involved in the biosynthesis of the enediyne antitumor antibiotic neocarzinostatin. Biochemistry 48, 9590-9598 (2009).
56 Zhang, K. et al. An engineered monolignol 4-O-methyltransferase depresses lignin biosynthesis and confers novel metabolic capability in Arabidopsis. Plant Cell 24, 3135-3152 (2012).
57 Zhao, Y. et al. Cloning, functional characterization, and catalytic mechanism of a bergaptol O-methyltransferase from Peucedanum praeruptorum Dunn. Front. Plant Sci. 7, 722 (2016).
58 Shimizu, T. et al. Purification and identification of naringenin 7-O-methyltransferase, a key enzyme in biosynthesis of flavonoid phytoalexin sakuranetin in rice. J. Biol. Chem. 287, 19315-19325 (2012).
59 Ycas, M. On earlier states of the biochemical system. J. Theor. Biol. 44, 145-160 (1974).
60 Jensen, R. A. Enzyme recruitment in evolution of new function. Annu. Rev. Microbiol. 30, 409-425 (1976).
61 Koh, J. C., Barbulescu, D. M., Salisbury, P. A. & Slater, A. T. Pterostilbene is a potential candidate for control of blackleg in canola. PLoS One 11, e0156186 (2016).
62 Osbourn, A. Secondary metabolic gene clusters: evolutionary toolkits for chemical innovation. Trends Genet. 26, 449-457 (2010).
63 Nutzmann, H. W. & Osbourn, A. Gene clustering in plant specialized metabolism. Curr. Opin. Biotechnol. 26, 91-99 (2014).
64 Chae, L., Kim, T., Nilo-Poyanco, R. & Rhee, S. Y. Genomic signatures of specialized metabolism in plants. Science 344, 510-513 (2014).
65 Goodstein, D. M. et al. Phytozome: a comparative platform for green plant genomics. Nucleic Acids Res. 40, D1178-1186 (2012).
66 Chang, Y.-S., Hsiao, Y.-C. & Huang, W.-L. Germplasm collection and agronomic traits analysis of Saccharum spontaneum L. in Taiwan. Crop, Environment & Bioinformatics 10, 98-110 (2013).
67 Lo, S.-C. et al. Phytoalexin accumulation in Sorghum: identification of a methyl ether of luteolinidin. Physiological and Molecular Plant Pathology 49, 21-31 (1996).
68 Reddy, S. P. et al. Evaluation of Sorghum [Sorghum bicolor (L.)] reference genes in various tissues and under abiotic stress conditions for quantitative real-time PCR data normalization. Front. Plant Sci. 7, 529 (2016).
69 Bottcher, A. et al. Lignification in sugarcane: biochemical characterization, gene discovery, and expression analysis in two genotypes contrasting for lignin content. Plant Physiol. 163, 1539-1557 (2013).
70 Labun, K. et al. CHOPCHOP v3: expanding the CRISPR web toolbox beyond genome editing. Nucleic Acids Res. 47, W171-W174 (2019).
71 Xing, H. L. et al. A CRISPR/Cas9 toolkit for multiplex genome editing in plants. BMC Plant Biol. 14, 327 (2014).
72 Liu, G., Campbell, B. C. & Godwin, I. D. Sorghum genetic transformation by particle bombardment. Methods Mol. Biol. 1099, 219-234 (2014).
73 Minor, W., Cymborowski, M., Otwinowski, Z. & Chruszcz, M. HKL-3000: the integration of data reduction and structure solution—from diffraction images to an initial model in minutes. Acta Crystallogr. D Biol. Crystallogr. D62, 859-866 (2006).
74 Kabsch, W. XDS. Acta Crystallogr. D Biol. Crystallogr. D66, 125-132 (2010).
75 Winn, M. D. et al. Overview of the CCP4 suite and current developments. Acta Crystallogr. D Biol Crystallogr. D67, 235-242 (2011).
76 Evans, P. R. & Murshudov, G. N. How good are my data and what is the resolution? Acta Crystallogr. D Biol. Crystallogr. D69, 1204-1214 (2013).
77 Cowtan, K. Completion of autobuilt protein models using a database of protein fragments. Acta Crystallogr. D Biol. Crystallogr. D68, 328-335 (2012).
78 McNicholas, S., Potterton, E., Wilson, K. S. & Noble, M. E. Presenting your structures: the CCP4 mg molecular-graphics software. Acta Crystallogr. D Biol. Crystallogr. D67, 386-394 (2011).
79 Keegan, R. M. & Winn, M. D. MrBUMP: an automated pipeline for molecular replacement. Acta Crystallogr. D Biol. Crystallogr. D64, 119-124 (2008).
80 McCoy, A. J. et al. Phaser crystallographic software. J. Appl. Crystallogr. 40, 658-674 (2007).
81 Vagin, A. A. et al. REFMAC5 dictionary: organization of prior chemical knowledge and guidelines for its use. Acta Crystallogr. D Biol. Crystallogr. D60, 2184-2195 (2004).
82 Emsley, P., Lohkamp, B., Scott, W. G. & Cowtan, K. Features and development of Coot. Acta Crystallogr. D Biol. Crystallogr. D66, 486-501 (2010).
83 Young, J. Y. et al. OneDep: unified wwPDB system for deposition, biocuration, and validation of macromolecular structures in the PDB archive. Structure 25, 536-545 (2017).
84 Chen, V. B. et al. MolProbity: all-atom structure validation for macromolecular crystallography. Acta Crystallogr. D Biol. Crystallogr. D66, 12-21 (2010).
85 Pettersen, E. F. et al. UCSF Chimera—a visualization system for exploratory research and analysis. J. Comput. Chem. 25, 1605-1612 (2004).
86 Pettersen, E. F. et al. UCSF ChimeraX: structure visualization for researchers, educators, and developers. Protein Sci. 30, 70-82 (2021).
87 Huey, R., Morris, G. M. & Forli, S. Using AutoDock 4 and AutoDock vina with AutoDockTools: a tutorial. The Scripps Research Institute Molecular Graphics Laboratory 10550, 92037 (2012).
88 Larkin, M. A. et al. Clustal W and Clustal X version 2.0. Bioinformatics 23, 2947-2948 (2007).
89 Madeira, F. et al. Search and sequence analysis tools services from EMBL-EBI in 2022. Nucleic Acids Res. (2022).
90 Kumar, S., Stecher, G., Li, M., Knyaz, C. & Tamura, K. MEGA X: Molecular Evolutionary Genetics Analysis across computing platforms. Mol. Biol. Evol. 35, 1547-1549 (2018).
91 Schneider, C. A., Rasband, W. S. & Eliceiri, K. W. NIH Image to ImageJ: 25 years of image analysis. Nat. Methods 9, 671-675 (2012).

Additional Sequences
AtOMT1 (Arabidopsis thaliana; NP_200227)
(SEQ ID NO: 5)
MGSTAETQLT PVQVTDDEAA LFAMQLASAS VLPMALKSAL ELDLLEIMAK
NGSPMSPTEI ASKLPTKNPE APVMLDRILR LLTSYSVLTC SNRKLSGDGV
ERIYGLGPVC KYLTKNEDGV SIAALCLMNQ DKVLMESWYH LKDAILDGGI
PFNKAYGMSA FEYHGTDPRF NKVENNGMSN HSTITMKKIL ETYKGFEGLT
SLVDVGGGIG ATLKMIVSKY PNLKGINFDL PHVIEDAPSH PGIEHVGGDM
FVSVPKGDAI FMKWICHDWS DEHCVKFLKN CYESLPEDGK VILAECILPE
TPDSSLSTKQ VVHVDCIMLA HNPGGKERTE KEFEALAKAS GFKGIKVVCD
AFGVNLIELL KKL
CtCAldOMT (Carthamus tinctorius; BAG71895)
(SEQ ID NO: 6)
MAHLEEEEAF LFAMQLASAS VLPMVLKSAI DLDLLEIIAK AGPGAHVSPA
YLAAQLPKAD NPEAAVMLDR ICRLLATYSV LTCTLKKLDH GDGDVERLYG
LAPVCKFLVK NEDGVSNAPL LLMNQDKILM ESWYHLKDAV LDGGIPFNKA
YGMSAFEYHG KDERFNKVFN SGMFNHSTMT MKKILDVYPG FNGVKTLVDV
GGGTGASLSM ILSKHPSIKG INFDLPHVIQ DATNYPGIEH VGGDMFESVP
KGDAIFMKWI CHDWSDAHCL KFLKNCYKAL PENGKVIVAE CILPETPDSS
LATKNVVHID VIMLAHNPGG KERTEKEFQA LAKAAGFKGF HKPCSALNTW
LMEFTK
CrCOMT (Catharanthus roseus; Q8W013)
(SEQ ID NO: 7)
MGSANPDNKN SMTKEEEEAC LSAMRLASAS VLPMVLKSAI ELDLLELIKK
SGPGAYVSPS ELAAQLPTQN PDAPVMLDRI LRLLASYSVL NCTLKDLPDG
GIERLYSLAP VCKFLTKNED GVSMAALLLM NQDKVLMESW YHLKDAVLEG
GIPFNKAYGM TAFEYHGKDP RENKVFNQGM SNHSTIIMKK ILEIYQGFQG
LKTVVDVGGG TGATLNMIVS KYPSIKGINF DLPHVIEDAP SYPGVDHVGG
DMFVSVPKGD AIFMKWICHD WSDAHCLKFL KNCHEALPEN GKVILAECLL
PEAPDSTLST QNTVHVDVIM LAHNPGGKER TEKEFEALAK GAGFRGFIKV
CCAYNSWIME LLK
HvCOMT (Hordeum vulgare; ABQ58825)
(SEQ ID NO: 8)
MGSIAAGADE DACMYALQLV SSSILPMTLK NAIELGLLET LMSAGGKFLT
PAEVAAKLPS TANPEAPDMV DRMLRLLASY KVVSCRTEES KDGRLSRRYG
AAPVCKYLTP NEDGVSMSAL ALMNQDKVLM ESWYYLKDAV LDGGIPFNKA
YGMSAFEYHG TDPRFNRVFN EGMKNHSIII TKKLLESYKG FEGLGTLVDV
GGGVGATVGA IIARYPAVKG INFDLPHVIS EAPAFPGVTH IGGDMFQKVP
SGDAILMKWI LHDWSDEHCA TLLKNCYDAL PAHGKVVLVE CILPVNPEAT
PEVQGVFHVD MIMLAHNPGG RERYEREFEA LAKGAGFAAM KTTYIYANAW
AIEFTK
MsCOMT (Medicago sativa; P28002)
(SEQ ID NO: 9)
MGSTGETQIT PTHISDEEAN LFAMQLASAS VLPMILKSAL ELDLLEIIAK
AGPGAQISPI EIASQLPTTN PDAPVMLDRM LRLLACYIIL TCSVRTQQDG
KVQRLYGLAT VAKYLVKNED GVSISALNLM NQDKVLMESW YHLKDAVLDG
GIPFNKAYGM TAFEYHGTDP RFNKVENKGM SDHSTITMKK ILETYTGFEG
LKSLVDVGGG TGAVINTIVS KYPTIKGINF DLPHVIEDAP SYPGVEHVGG
DMFVSIPKAD AVFMKWICHD WSDEHCLKFL KNCYEALPDN GKVIVAECIL
PVAPDSSLAT KGVVHIDVIM LAHNPGGKER TQKEFEDLAK GAGFQGFKVH
CNAFNTYIME FLKKV
MsIOMT (Medicago sativa; AAC49927)
(SEQ ID NO: 10)
MASSINGRKP SEIFKAQALL YKHIYAFIDS MSLKWAVGMN IPNIIHNHGK
PISLSNLVSI LQVPSSKIGN VRRLMRYLAH NGFFEIITKE EESYALTVAS
ELLVRGSDLC LAPMVECVLD PTLSGSYHEL KKWIYEEDLT LFGVTLGSGF
WDFLDKNPEY NTSFNDAMAS DSKLINLALR DCDFVEDGLE SIVDVGGGTG
TTAKIICETF PKLKCIVEDR PQVVENLSGS NNLTYVGGDM FTSIPNADAV
LLKYILHNWT DKDCLRILKK CKEAVTNDGK RGKVTIIDMV INEKKDENQV
TQIKLLMDVN MACLNGKERN EEEWKKLFIE AGFQHYKISP LTGFLSLIEI
YP
OsCAldOMT1 (Oryza sativa; Q6ZD89)
(SEQ ID NO: 11)
MGSTAADMAA AADEEACMYA LQLASSSILP MTLKNAIELG LLETLQSAAV
AGGGGKAALL TPAEVADKLP SKANPAAADM VDRMLRLLAS YNVVRCEMEE
GADGKLSRRY AAAPVCKWLT PNEDGVSMAA LALMNQDKVL
MESWYYLKDA VLDGGIPFNK AYGMTAFEYH GTDARFNRVF NEGMKNHSVI
ITKKLLDLYT GFDAASTVVD VGGGVGATVA AVVSRHPHIR GINYDLPHVI
SEAPPFPGVE HVGGDMFASV PRGGDAILMK WILHDWSDEH CARLLKNCYD
ALPEHGKVVV VECVLPESSD ATAREQGVFH VDMIMLAHNP GGKERYEREF
RELARAAGFT GFKATYIYAN AWAIEFTK
OsNOMT (Oryza sativa; QOIP69)
(SEQ ID NO: 12)
MVSPVVHRHA AGGGSGGDDD DQACMYALEL LGGSVVSMTL KAAIELGLVD
ELLAAAGAAV TAEELAARLR LPAAVAAAAA VDRMLRLLAS YGVVRCATEA
GPDGKARRSY AAAPVCKWLA AGSSSGEGSM APLGLLNLDK VFMENWYYLK
EAVSEGGTAF DKAYGTSLFQ YLGQDGNEPS NTLFNQAMAS HSVVITNKLL
QFFRGFDAGA GVDVLVDVGG GVGATLRMIT ARHPHLRGVN YDLPHVIAQA
PPVEGVEHIG GSMFDHVPSG SAILLKWILH LWGDEECVKI LKNCYKALPA
KGKVILVEYV LPASPEATLA AQEAFRLDVM MLNRLAGGKE RTQQEFTDLA
VDAGFSGDCK PTYIFTNVWA LEFTK
PvCOMT (Panicum virgatum; ADX98508)
(SEQ ID NO: 13)
MGSTATDVAA AADEEACMYA LQLASSSILP MTLKNAIELG LLEVLQKDPA
AALAPEEVVA QLPVAPANPD AAAMVDRMLR LLASYDVVRC QMEEGKDGRY
SRRYAAAPVC KWLTPNEDGV SMAALALMNQ DKVLMESWYY LKDAVLEGGI
PFNKAYGMTA FEYHGTDPRF NRVFNEGMKN HSVIITKKLL EFYAGFEGVG
TLVDVGGGVG ATLHAITSRY PGIRGVNFDL PHVISEAPPF PGVEHVGGDM
FKAVPAGDAI LMKWILHDWS DAHCAAILKN CYDALPAGGK VIAVECILPV
NPEATPKAQG VFHVDMIMLA HNPGGKERYE REFEELAKGA GFTGFKATYI
YANAWAIEFT K
PsPMT2 (Pinus sylvestris; AQX17823)
(SEQ ID NO: 14)
MNMQSVKDEE ALRASALGLA FSLETPFLLK CAIRLKIPDI ISKAGPDVSL
SVHQIAAQLP SEDPDMGALS RILTYLSTMG ILQAIVPPEG VNAPMNIRYG
LTNLTKTYFT SEDISSRSLV PFVLLQTHPL YVTAWDNIHE RVLHGGDNFK
NSSGNGKDFW NFAAGEPEFN AIFNAGMVSV TKATITYVLA VYDGFKDINT
LVDVGGGRGE ALSLITEAHP HIRAINFDLP QVIATAPTIP GVQHMSGNLF
ESAPSADAIF MKNFLHSWND EDCIKLLNNC HQALPEKGKL ILSEAILDLT
EGSDMIGSAN VLDAVMLNCL PGGGERTRKQ WNDLLQAAGF SISKIVGRNG
TLTKVIEAIK S
PtAEOMT (Pinus taeda; Q43096)
(SEQ ID NO: 15)
MDSNMNGLAK SNGCEISRDG FFESEEEELQ GQAEAWKCTF AFAESLAVKC
VVLLGIPDMI AREGPRATLS LCEIVANLPT ESPDAACLFR IMRFLVAKGI
FPASKSARRR AFETRYGLTP ASKWLVKGRE LSMAPMLLMQ NDETTLAPWH
HFNECVLEGG VAFQKANGAE IWSYASDHPD FNNLFNNAMA CNARIVMKAI
LSKYQGFHSL NSLVDVGGGT GTAVAEIVRA YPFIRGINYD LPHVVATASS
LSGVQHVGGD MFETVPTADA IFMKWIMHDW NDEDCIKILK NCRKAIPDTG
KVIIVDVVLD ADQGDNTDKK RKKAVDPIVG TVFDLVMVAH SSGGKERTEK
EWKRILLEGG FSRYNIIEIP ALQSVIEAFP R
PtCOMT (Populus tremuloides; AAB61731)
(SEQ ID NO: 16)
MGSTGETQMT PTQVSDEEAH LFAMQLASAS VLPMILKTAI ELDLLEIMAK
AGPGAFLSTS EIASHLPTKN PDAPVMLDRI LRLLASYSIL TCSLKDLPDG
KVERLYGLAP VCKFLTKNED GVSVSPLCLM NQDKVLMESW YYLKDAILDG
GIPFNKAYGM TAFEYHGTDP RFNKVFNKGM SDHSTITMKK ILETYKGFEG
LTSLVDVGGG TGAVVNTIVS KYPSIKGINF DLPHVIEDAP SYPGVEHVGG
DMFVSVPKAD AVFMKWICHD WSDAHCLKFL KNCYDALPEN GKVILVECIL
PVAPDTSLAT KGVVHVDVIM LAHNPGGKER TEKEFEGLAK GAGFQGFEVM
CCAFNTHVIE FRKKA
RhOOMT (Rosa hybrid; AAM23004)
(SEQ ID NO: 17)
MERLNSFRHL NQKWSNGEHS NELLHAQAHI WNHIFSFINS MSLKSAIQLG
IPDIINKHGY PMTLSELTSA LPIHPTKSHS VYRLMRILVH SGFFAKKKLS
KTDEEGYTLT DASQLLLKDH PLSLTPYLTA MLDPVLTNPW NYLSTWFQND
DPTPFDTAHG MTFWDYGNHQ PSIAHLENDA MASDARLVTS VIINDCKGVF
EGLESLVDVG GGTGTLAKAI ADAFPHIECT VLDLPHVVAD LQGSKNLKYT
GGDMFEAVPP ADTVLLKWIL HDWSDEECIK ILERSRVAIT GKEKKGKVII
IDMMMENQKG DEESIETQLF FDMLMMALVG GKERNEKEWA KLFTDAGFSD
YKITPISGLR SLIEVYP
SICOMT (Solanum lycopersicum; XP_004235028)
(SEQ ID NO: 18)
MGSTSLTQTE DEAFLFAMQL ASASVLPMVL KSAVELELLE LMAKAGPGAS
ISPAELASQL PCKNPDAPVM LDRMLRLLAA YSVLNCTLRT LPDGRVERLY
SLAPVCKFLT KNADGVSVAP LLLMNQDKVL MQSWYHLKDA VLDGGIPFNK
AYGMTAFEYH GTDPRFNKVF NRGMSDHTTL SIKKILEDYK GFEGLNSIVD
VGGGTGATVS MIVSKYPSIK GINFDLPHVI EDAPAYPGVE HIGGDMFVSV
PKADAIFMKW ICHDWSDEHC LKFLKNCYEA VPANGKVIIA ECLLPEVPDT
SSSTKNTVHV DVIMLAHNPG GKERTEKEFE ALAKGAGENG FTKASCAYNT
WIMEFTK
SbNOMT (Sorghum bicolor; XP_002465645)
(SEQ ID NO: 19)
MACTTAASLQ HDKANDDEAC MYAQELLFSF IVPMTLKAVI ELGLIDYLLA
ADGRSVTPEE LAAEWPQSAE AAAAVDRMMR LLASHSVVRC TTEVGPDGKA
RRSYAAAPVC KWLATRNAGG QGSLAPMGLM NLNKAFMETW
YFMKEAVTEG ATPTEKAYGM PLFEHLGSDE ASNTLFNQAM AGHSEMIIKK
LLEVYRGFEG VDVLVDVGGG TGSTLRMVTA QYKHLRGVNY DLPHVIAQAP
PVQGVEHVGG SMFEYIPSGN AILLKWILHL WRDDECVKIL KNCHRALPAN
GKVIVVEYVL PASPEPTQVA QVSLLLDVAM LNRLRGAKER TEQEFAQLAA
EAGFSGGCRA TYVFASAWAL EFTK
SbOMT1 (Sorghum bicolor; ABP01563)
(SEQ ID NO: 20)
MASYTSTSGQ FAVGKVAAAN QDDETCMHAL KLLGGLAVPF TIKAVIELGI
MDLLLAADRA MTAEALTAAL LCPAPAPAAA AAMVDRMLRF LASHGVVRCA
TESEELGSDD GKSCRRYAAA PVCKWFARGG GVESVVPMGF WMTSTTNMET
WHNIKDGVLA GETPFDKAYG MPVFEYLGAN GTMNTLFNEA MASHSMIITK
RLLEVFRGFE NYSVLVDVGG GNGTTMQMIR SQYENISGIN YDLPHVIAQA
SPIEGVEHVA GNMEDNIPRG DAIILKWILH NWGDKECVKI LKNCYTALPV
NGTVIILEYI LPETPEETLA SQLAFDFDLG MMLFFGASGK ERTEKELLEL
AREAGFSGDY TATYIFANVW AHEFTK
SbOMT3 (Sorghum bicolor; ABP01564)
(SEQ ID NO: 21)
MVLISEDSRE LLQAHVELWN QTYSFMKSVA LAVALDLHIA DAIHRRGGAA
TLSQILGEIG VRPCKLPGLH RIMRVLTVSG TFTIVQPSAE TMSSESDGRE
PVYKLTTASS LLVSSESSAT ASLSPMLNHV LSPFRDSPLS MGLTAWFRHD
EDEQAPGMCP FTLMYGTTLW EVCRRDDAIN ALFNNAMAAD SNFLMQILLK
EFSEVFLGID SLVDVAGGVG GATMAIAAAF PCLKCTVLDL PHVVAKAPSS
SIGNVQFVGG DMFESIPPAN VVLLKWILHD WSNDECIKIL KNCKQAIPSR
DAGGKIIIID VVVGSDSSDT KLLETQVIYD LHLMKIGGVE RDEQEWKKIF
LEAGFKDYKI MPILGLRSII ELYP
SbOMT4 (Sorghum bicolor; XP_002443937)
(SEQ ID NO: 22)
MGSYTTRVHA VEKAANNQDD ETCMHALMLL GGLAVPCTIK AVIELGIMDL
LLAADRAMTA EELTARLPCP AAATAAAMVD RMLRFLASHG VVRCAAATKE
SSELGSDGGK SCRRYAAAPV CRWFTRSGGV ESVVPMGLWM TGKTVLETWY
NIKEAVLEGE TPFDRAYGQP FFEYLGENGT VNTLFDEAMA NHSTIITKRL
VEVFRGFENY SVLVDVGGNK GTTLQMIRSQ YENISGINYD LPRVIAQAPP
IEGVEHVGGN MFDNVPRGDA IILKWILHDW GDKDCVKILK NCYAALPVNG
TMIILEYILP ETPEETLTSQ LAFNFDFGMM LMYGAKGKER TEKELSELAR
EAGFSGDCTA TYIFASIWAL EFTK
SbSTS1 (Sorghum bicolor; XP_002445139)
(SEQ ID NO: 23)
MTTGKVTLEA VRKAQRAEGP ATVLAIGTAT PANCVYQADY PDYYFRVTKS
EHLTDLKEKF KRICHKSMIR KRYMHLTEDI LEENPNMSSY WAPSLDARQD
ILIQEIPKLG AEAAEKALKE WGQPRSRITH LVFCTTSGVD MPGADYQLIK
LLGLCPSVNR AMMYHQGCFA GGMVLRLAKD LAENNRGARV LIVCSEITVV
TFRGPSESHL DSLVGQALFG DGAAAVIVGA DPSEPAERPL FHLVSASQTI
LPDSEGAIEG HLREVGLTFH LODRVPQLIS MNIERLLEDA FAPLGISDWN
SIFWVAHPGG PAILNMVEAK VGLDKARMCA TRHILAEYGN MSSVCVLFIL
DEMRNRSAKD GHTTTGEGME WGVLFGFGPG LTVETIVLHS VPITTVAA
TaCOMT1 (Triticum aestivum; AAP23942)
(SEQ ID NO: 24)
MGSTAADMAA SADEEACMYA LQLVSSSILP MTLKNAIELG LLETLVAAGG
KLLTPAEVAA KLPSTANPAA ADMVDRMLRL LASYNVVSCT MEEGKDGRLS
RRYRAAPVCK FLTPNEDGVS MAALALMNQD KVLMESWYYL KDAVLDGGIP
FNKAYGMSAF EYHGTDPRFN RVFNEGMKNH SIIITKKLLE VYKGFEGLGT
IVDVGGGVGA TVGAITAAYP AIKGINFDLP HVISEAQPFP GVTHVGGDMF
QKVPSGDAIL MKWILHDWSD EHCATLLKNC YDALPAHGKV VLVECILPVN
PEATPKAQGV FHVDMIMLAH NPGGRERYER EFEALAKGAG FKAIKTTYIY
ANAFAIEFTK
TaCOMT2 (Triticum aestivum; Q38J50)
(SEQ ID NO: 25)
MGSIAAGADE DACMYALQLV SSSILPMTLK NAIELGLLET LMAAGGKFLT
PAEVAAKLPS AANPEAPDMV DRMLRLLASY NVVSCRTEDG KDGRLSRRYG
AAPVCKYLTP NEDGVSMSAL ALMNQDKVLM ESWYYLKDAV LDGGIPFNKA
YGMSAFEYHG TDPRFNRVFN EGMKNHSIII TKKLLESYKG FEGLGTLVDV
GGGVGATVAA ITAHYPTIKG INFDLPHVIS EAPPFPGVTH VGGDMFQKVP
SGDAILMKWI LHDWSDEHCA TLLKNCYDAL PAHGKVVLVE CILPVNPEAT
PKAQGVFHVD MIMLAHNPGG RERYEREFEA LAKGAGFAAM KTTYIYANAW
AIEFTK
VvCOMT (Vitis vinifera; NP_001268100)
(SEQ ID NO: 26)
MESTLAFNSG SNSMNQSFSS SAEFNSPVPE TIPKSEEDTF VFATLLTSAS
VLPMALKSAL ELDLLEIIAK AGPGAFVSTS EIAAKITKRN PKAPVMLDRI
LRLLATYDVV KCSLRDSPDG GVERLYGLGP VCKYFTTNED GVSVAPLLLM
NQDKVPMQSK RYHLKDAVLD GGIPFNKAYG MTDFEYHGTE PRENKVENNG
VSGHPTITMK KILEAYKGFE GLTSIVDVGG GTGATLNMII SKYPTIKGIN
FDLPHVIDDA PSYPGVEHVG GDMFVSVPKG DAIFMKWMCY EWDDAHCLKF
LENCYQALPD NGKVIVAECI LPVVPDTSLA TKSAVHIDVI MLAYNTGGKA
RTEKEFEALA KGAGFQGFKV VCCAFNSWIM EFCKTA
VvROMT (Vitis vinifera; CAQ76879)
(SEQ ID NO: 27)
MDLANGVISA ELLHAQAHVW NHIFNFIKSM SLKCAIQLGI PDIIHNHGKP
MTLPELVAKL PVHPKRSQCV YRLMRILVHS GFLAAQRVQQ GKEEEGYVLT
DASRLLLMDD SLSIRPLVLA MLDPILTKPW HYLSAWFQND DPTPFHTAHE
RSFWDYAGHE PQLNNSFNEA MASDARLLTS VLLKEGQGVF AGLNSLVDVG
GGTGKVAKAI ANAFPHLNCT VLDLPHVVAG LQGSKNLNYF AGDMFEAIPP
ADAILLKWIL HDWSDEECVK ILKRCREAIP SKENGGKVII IDMIMMKNQG
DYKSTETQLF FDMTMMIFAP GRERDENEWE KLFLDAGFSH YKITPILGLR
SLIEVYP
ZmCOMT (Zea mays; Q06509)
(SEQ ID NO: 28)
MGSTAGDVAA VVDEEACMYA MQLASSSILP MTLKNAIELG LLEVLQKEAG
GGKAALAPEE VVARMPAAPG DPAAAAAMVD RMLRLLASYD
VVRCQMEDRD GRYERRYSAA PVCKWLTPNE DGVSMAALAL MNQDKVLMES
WYYLKDAVLD GGIPFNKAYG MTAFEYHGTD SRFNRVFNEG MKNHSVIITK
KLLDFYTGFE GVSTLVDVGG GVGATLHAIT SRHPHISGVN FDLPHVISEA
PPFPGVRHVG GDMFASVPAG DAILMKWILH DWSDAHCATL LKNCYDALPE
NGKVIVVECV LPVNTEATPK AQGVFHVDMI MLAHNPGGKE RYEREFRELA
KGAGFSGFKA TYIYANAWAI EFIK
ZmNOMT (Zea mays; XP_020399799)
(SEQ ID NO: 29)
MACTTAASLQ HDRANDDEAC MYAQELLSCF VVPMTLKAVI ELGLIDDLLA
ADGRFVTPEE LAARWARPAE AAAAVDRMLR FLASHSVVRC TTEAAGPDGR
ARRSYAAAPV CKWLIARNGT GQGSWAPIGL MNLNKGFMET WYYMKDAVAE
GATPTEKAYG MPLFEHLGSD EALNTLFNQA MAGHSEIVIS KLLEVYRGFE
GVDVLVDVGG GTGSTLRMVT AQYKHLRGVN YDLPHVIAQA PPVQGVEHAG
GSMFEYIPSG NAILLKWILH LWRDEECIKI LKNCHRALPA NGKVIVVECV
LPASPEPTQV AQGALLLDVV MLNRLRGAKE RTEREFTELA AEAGFSGGCR
ATYVFTGAWA LEFTK
ShOMTI (Sorghum halepense; GGDZ01007645)
(SEQ ID NO: 30)
atgggcagct atactaccag agtccacgcc gtagagaaag cagctaacaa
ccaagatgat gagacgtgca tgcatgcttt gatgctcctt ggcggattgg
ccgtaccttg taccatcaag geggtcateg agctcggcat catggacctt
ctcctcgceg cggacegcgc catgaccgeg gaggagctca cggcaaggtt
gccatgtcct gctgctgcta ctgctgctgc tatggtcgac cgcatgctcc
gettcctage ttcgcacggc gtggtcaggt gegccgccgc caccaaggag
tcgtcggagc tgggctccga tggtggcaag agctgccggc gctacgcggc
ggcgccggtg tgcaggtggt ttacaaggag cggcggcgtg gagtcggtgg
ttccaatggg gttgtggatg accggcaaga ccgtcttgga gacctggtac
aacataaaag aagcagtgtt agagggagaa acaccatttg acagagcata
cggccagcca ttttttgagt accttggtga aaacgggaca gtgaacacgt
tgttcgatga ggcaatggcg aaccattcga caatcataac gaagaggctg
gtcgaggtct tccgtggctt tgagaattac agtgtgctcg tcgatgtcgg
cggcaacaaa ggcaccacgt tgcaaatgat tagaagtcag tatgagaata
ttagtggcat caactacgac cttcctcgtg taattgcgca ggcacctcca
attgaaggtg tggagcatgt tggtggcaac atgtttgata atgttccacg
tggagatgca attatcctaa agtggattct tcacgattgg ggcgacaagg
actgcgtcaa gatcctaaag aattgctatg cagctctccc ggtgaacggg
acgatgatca ttctggagta catcctccca gagacaccag aagaaacact
aacategcaa ctagcgttca acttcgattt cgggatgatg ctcatgtacg
gogccaaagg taaggagaga acagagaaag agttgtcgga gctcgccaga
gaggccggct tctctggaga ctgcacggct acatacatct tegccagcat
ctgggccctt gaattcacaa agtaa
ShSOMT (Sorghum halepense; GGDZ01140964)
(SEQ ID NO: 31)
tcgtcctcta tctgtggcca ccaactgctg tgctatttga gtacacagat
acagatgggc agctacgaca gcagcagtag tagtagtaat gactcatcag
cacggaacga ggaggacgag tcgtgcatgt ttgctttgaa gctcctcggc
gggttcgccg tacctttcac catcaaggcg gtgattgagc tgggcgtcat
ggaccagctc ctgactgccg aacgcgcgat gagcgcggag gagctggtgg
cagcagcagt ggcagcacag ctgccacggc ccgaggtagc ctgcaccatg
gtggaccgcc tgctccgttt ccttgcctcg cacagcgtag tccggtgcac
gaccgaggtg gtggtgggca cggacgacgc caccaccacc acctgetgcc
gccggagcta cgcegcgtca cccgtctgca agtggttcgc caggaacggc
gtcgaggatt cggtgcttcc gcttgggatg atgatcctaa acaagacatt
cctggacagc tggcaaaaca ttacggatgc agtgttggaa ggagcagcac
catttgagaa aacctacggg atgccaatgt tegagtacct aagtacaaac
ggaccattga acacggtgtt ccacgaggca atggcaaatc attcgatgat
tataaccaag aaactgctca agttcttccg cggcttcgaa ggccttgatg
tgctggtcga cgtaggcggc ggcaacggca ccacgttgca aatgattaga
ggtcaatata agaatatgag aggcataaac tacgaccttc ctcatgtcat
tgcgcaggct gcaccagttg aaggtgtgga acatgtaggt ggcagcatgt
tcgataatat tccacgegga aatgcagttc tgctcaagtg gattcttcat
gattgggacg acaaggcgtg catcaagatc ctaaagaatt gctatacagc
tctccatgtg agaggcaagg tgatcgttct ggagtacgtt gttccggatg
aaccagaacc tactcttgca gctcagggtg ccttcgaact ggacctcacc
atgctggtca cgtttggcag tggtaaagag aggacacaga gggagttctc
cgagctcgcc atggaggccg gcttctctag agagtttaag gctacgtata
tctttgccaa cgtctgggcc cttgagttca caaagtaa
SsCOMT (Saccharum spontaneum)
(SEQ ID NO: 32)
MGSTAEDVAA VADEEACMYA MQLASASILP MTLKNALELG LLEVLQAEAP
AGKALAPEEV VARLPVAPTN PDAADMVDRM LRLLASYDVV KCQMEDKDGK
YERRYSAAPV GKWLTPNEDG VSMAALTLMN QDKVLMESRY YLKDAVLDGG
IPFNKAYGMT AFEYHGTDPR FNRVFNEGMK NHSVIITKKL LEFYTGFEGV
STLVDVGGGI GATLHAITSH HPQIKGINFD LPHVISEAPP FPGVQHVGGD
MFKSVPAGDA ILMKWILHDW SDAHCATLLK NCYDALPENG KVIVVECVLP
VNTEAVPKAQ GVFHVDMIML AHNPGGRERY EREFHDLAKG AGFSGFKATY
IYANAWAIEF IK
SsSTS (Saccharum spontaneum)
(SEQ ID NO: 33)
MTGKVTLGAV RKAQRAEGSA AVLAIGTATP ANCVYQADYP DYYFRVTKSE
HLTDLKEKFK RICHKSMITK RYMHLTEGFL QENPNMSSYS APSLDARQDI
LIEEVPKLGA AAAEKALKEW GQPRSQITHL VFCTTSGVDM PGADYQLIKL
LGLSLSVNRA MMYHQGCFAG GMVLRLAKDL AENNRGARVL IVCSEITAVT
FRGPSESHLD SLVGQALFGD GAAAVIVGAD PSAAEWPLFQ LVSASQTILP
DSEGAIEGHL REVGLTFHLQ DRVPQLISTN IERLLEDAFT PLGISDWNSI
FWVAHPGGPA ILNMVEAKAG LDKARLCATR HILAEYGNMS SACVLFILDE
MRNKSAEDGH TTTGEGMEWG VLFGFGPGLT VETIVLQSVP IITV
ShOMT1 (Sorghum halepense)
(SEQ ID NO: 34)
MGSYTTRVHA VEKAANNQDD ETCMHALMLL GGLAVPCTIK AVIELGIMDL
LLAADRAMTA EELTARLPCP AAATAAAMVD RMLRFLASHG VVRCAAATKE
SSELGSDGGK SCRRYAAAPV CRWFTRSGGV ESVVPMGLWM TGKTVLETWY
NIKEAVLEGE TPFDRAYGQP FFEYLGENGT VNTLFDEAMA NHSTIITKRL
VEVFRGFENY SVLVDVGGNK GTTLQMIRSQ YENISGINYD LPRVIAQAPP
IEGVEHVGGN MFDNVPRGDA IILKWILHDW GDKDCVKILK NCYAALPVNG
TMIILEYILP ETPEETLTSQ LAFNFDFGMM LMYGAKGKER TEKELSELAR
EAGFSGDCTA TYIFASIWAL EFTK
ShSOMT (Sorghum halepense)
(SEQ ID NO: 35)
MGSYDSSSSS SNDSSARNEE DESCMFALKL LGGFAVPFTI KAVIELGVMD
QLLTAERAMS AEELVAAAVA AQLPRPEVAC TMVDRLLRFL ASHSVVRCTT
EVVVGTDDAT TTTCCRRSYA ASPVCKWFAR NGVEDSVLPL GMMILNKTFL
DSWQNITDAV LEGAAPFEKT YGMPMFEYLS TNGPLNTVFH EAMANHSMII
TKKLLKFFRG FEGLDVLVDV GGGNGTTLQM IRGQYKNMRG INYDLPHVIA
QAAPVEGVEH VGGSMFDNIP RGNAVLLKWI LHDWDDKACI KILKNCYTAL
HVRGKVIVLE YVVPDEPEPT LAAQGAFELD LTMLVTFGSG KERTQREFSE
LAMEAGFSRE FKATYIFANV WALEFTK
MsIOMT (Medicago sativa; P93324)
(SEQ ID NO: 82)
MGNSYITKED NQISATSEQT EDSACLSAMV LTTNLVYPAV LNAAIDLNLF
EIIAKATPPG AFMSPSEIAS KLPASTQHSD LPNRLDRMLR LLASYSVLTS
TTRTIEDGGA ERVYGLSMVG KYLVPDESRG YLASFTTFLC YPALLQVWMN
FKEAVVDEDI DLFKNVHGVT KYEFMGKDKK MNQIFNKSMV DVCATEMKRM
LEIYTGFEGI STLVDVGGGS GRNLELIISK YPLIKGINFD LPQVIENAPP
LSGIEHVGGD MFASVPQGDA MILKAVCHNW SDEKCIEFLS NCHKALSPNG
KVIIVEFILP EEPNTSEESK LVSTLDNLMF ITVGGRERTE KQYEKLSKLS
GFSKFQVACR AFNSLGVMEF YK
MsCOMT (Medicago sativa; P28002)
(SEQ ID NO: 83)
MGSTGETQIT PTHISDEEAN LFAMQLASAS VLPMILKSAL ELDLLEIIAK
AGPGAQISPI EIASQLPTTN PDAPVMLDRM LRLLACYIIL TCSVRTQQDG
KVQRLYGLAT VAKYLVKNED GVSISALNLM NQDKVLMESW YHLKDAVLDG
GIPFNKAYGM TAFEYHGTDP RFNKVENKGM SDHSTITMKK ILETYTGFEG
LKSLVDVGGG TGAVINTIVS KYPTIKGINF DLPHVIEDAP SYPGVEHVGG
DMFVSIPKAD AVFMKWICHD WSDEHCLKFL KNCYEALPDN GKVIVAECIL
PVAPDSSLAT KGVVHIDVIM LAHNPGGKER TQKEFEDLAK GAGFQGFKVH
CNAFNTYIME FLKKV
MtIOMT (Medicago truncatula; Q29U70)
(SEQ ID NO: 84)
MAFSTNGSEE SELYHAQIHL YKHVYNFVSS MALKSAMELG IADAIHNHGK
PMTLSELASS LKLHPSKVNI LHRFLRLLTH NGFFAKTIVK GKEGDEEEEI
AYSLTPPSKL LISGKPTCLS SIVKGALHPS SLDMWSSSKK WFNEDKEQTL
FECATGESFW DFLNKDSESS TLSMFQDAMA SDSRMFKLVL QENKRVFEGL
ESLVDVGGGT GGVTKLIHEI FPHLKCTVFD QPQVVGNLTG NENLNFVGGD
MFKSIPSADA VLLKWVLHDW NDEQSLKILK NSKEAISHKG KDGKVIIIDI
SIDETSDDRG LTELQLDYDL VMLTMFLGKE RTKQEWEKLI YDAGFSSYKI
TPISGFKSLI EVYP
MtIOMT3 (Medicago truncatula; Q06YR3)
(SEQ ID NO: 85)
MASSINNRKP SEIFKAQALL YKNMYAFVDS MSLKWSIEMN IPNIIHNHGK
PITLSNLVSI LQIPSTKVDN VQRLMRYLAH NGFFEIITNQ ELENEEEAYA
LTVASELLVK GTELCLAPMV ECVLDPTLST SFHNLKKWVY EEDLTLFAVN
LGCDLWEFLN KNPEYNTLYN DALASDSKMI NLAMKDCNLV FEGLESIVDV
GGGNGTTGKI ICETFPKLTC VVFDRPKVVE NLCGSNNLTY VGGDMFISVP
KADAVLLKAV LHDWTDKDCI KILKKCKEAV TSDGKRGKVI VIDMVINEKK
DENQLTQIKL LMNVTISCVN GKERNEEEWK KLFIEAGFQD YKISPFTGLM
SLIEIYP
ScNOMT (Streptomyces carzinostaticus; Q84HC8)
(SEQ ID NO: 86)
MGKRAAHIGL RALADLATPM AVRVAATLRV ADHIAAGHRT AAEIASAAGA
HADSLDRLLR HLVAVGLFTR DGQGVYGLTE FGEQLRDDHA AGKRKWLDMN
SAVGRGDLGF VELAHSIRTG QPAYPVRYGT SFWEDLGSDP VLSASFDTLM
SHHLELDYTG IAAKYDWAAL GHVVDVGGGS GGLLSALLTA HEDLSGTVLD
LQGPASAAHR RFLDTGLSGR AQVVVGSFFD PLPAGAGGYV LSAVLHDWDD
LSAVAILRRC AEAAGSGGVV LVIEAVAGDE HAGTGMDLRM LTYFGGKERS
LAELGELAAQ AGLAVRAAHP ISYVSIVEMT AL
LpCOMT (Loium perenne; Q9ZTU2)
(SEQ ID NO: 87)
MGSTAADMAA SADEDACMFA LQLASSSVLP MTLKNAIELG LLEILVAAGG
KSLTPTEVAA KLPSAANPEA PDMVDRILRL LASYNVVTCL VEEGKDGRLS
RSYGAAPVCK FLTPNEDGVS MAALALMNQD KVLMESWYYL KDAVLDGGIP
FNKAYGMSAF EYHGTDPREN RVFNEGMKNH SIIITKKLLE LYHGFEGLGT
LVDVGGGVGA TVAAIAAHYP TIKGVNFDLP HVISEAPQFP GVTHVGGDMF
KEVPSGDTIL MKWILHDWSD QHCATLLKNC YDALPAHGKV VLVQCILPVN
PEANPSSQGV FHVDMIMLAH NPGGRERYER EFQALARGAG FTGVKSTYIY
ANAWAIEFTK
CHIEOMT (Clarkia breweri; 004385)
(SEQ ID NO: 88)
MGSTGNAEIQ IIPTHSSDEE ANLFAMQLAS AAVLPMALKA AIELDVLEIM
AKSVPPSGYI SPAEIAAQLP TTNPEAPVML DRVLRLLASY SVVTYTLREL
PSGKVERLYG LAPVCKFLTK NEDGVSLAPF LLTATDKVLL EPWFYLKDAI
LEGGIPFNKA YGMNEFDYHG TDHRFNKVFN KGMSSNSTIT MKKILEMYNG
FEGLTTIVDV GGGTGAVASM IVAKYPSINA INFDLPHVIQ DAPAFSGVEH
LGGDMFDGVP KGDAIFIKWI CHDWSDEHCL KLLKNCYAAL PDHGKVIVAE
YILPPSPDPS IATKVVIHTD ALMLAYNPGG KERTEKEFQA LAMASGFRGF
KVASCAFNTY VMEFLKTA
PIOMT (Planctopirus limnophila; D5STZ7)
(SEQ ID NO: 89)
MAVKDALRFP PTDVTPIFDL FRGNFATELL AASVAHLHVF DILNESPLSL
DELQRRLVLS ERATQVLVTG LCAMQLLTKR LAGEIDLTPL ARNHLVTTSP
FSVGGYISLA AQSAGTLALV ERLKSDRPEG AESEQGAAFI FREGSESAMD
REDSARFLTL SLAGRAWNVA PRFADVLPAG QPGKILKDNS GSSGRVLLDV
AGGSGIYTMA VLQKYPTWRG IIFDRPEVLK IAAELAEQTG VRDRLELHAG
DMWVDPFPPA DDILLSNVLH DWDRPQCARL VAKATSGLPE GGRLLIHDVL
LNSDLTGPLE IALYSLALFS LTEGRAYSLE EYRGWIAGAD LKYVDCIPTS
AHGHLILSEK V
TINOMT (Thalictrum flavum; Q5C9L7)
(SEQ ID NO: 90)
MEMINKENLS SQAKLWNFIY GFADSLVLKS AVQLDLANII HNHGSPMTLS
ELSLHLPSQP VNQDALYRVL RYLVHMKLFT KSSIDGELRY GLAPPAKFLV
KGWDKCMLGA ILTITDKDFM APWHYLKEGI LNDGSTSTAF EKALGTNIWD
YMAEHPEKNQ LFNEGMANDT RLIMSALVKE CSSMFDGITT IVDVGGGTGT
AVRNIAKAFP HIKCTVYDLP HVIADSPGYT EINSIQGDMF KYIPNADAIM
MKCILHDWDD KECIEILKRC KDAVPRDGGK VIIIDIILDV KSEHPYTKMR
LTLDLDMMLN TGGKERTEEE WKKLIHDAGY KGYKITHISA VQSVIEAYPY
KpBOMT (Kitagawia praeruptora; A0A166U5H3)
(SEQ ID NO: 91)
MAGMKTSPSQ DEEACVLAIQ LATSTVLPMI LKSAIELDIL NTISKAGPGN
YLSPSDLASK LLMSNPHAPI MLERILRVLA TYKVLGCKPS ELSDGEVEWL
YCWTPVCKFL SNNEDGASIA PLLLVHQDQV PMKSWYHLTD AILDGGTAFN
KAYGMNIFDY ASQDPQFNKV FNRSMAGHST ITMKKILETY NGFEGLKSIV
DVGGGSGATL NMIISKYPTI KGINFDLPHV VGDSPIHPGV EHVGGDMFAS
VPKGDAIFLK WIFHSWSDED CLRILKNCYE ALADNKKVIV AEFIIPEVPG
GSDDATKSVV HLDAVMLAYV PGGKERTEKE FEALATSAGF KSFRKVCCAF
NTWIMEFSK
PsSOMT (Papaver somniferum; I3V6A7)
(SEQ ID NO: 92)
MATNGEIFNT YGHNRQTATV TKITASNESS NGVCYLSETA NLGKLICIPM
ALRAAMELNV FQLISKFGTD AKVSASEIAS KMPNAKNNPE AAMYLDRILR
LLGASSILSV STTKKSINRG GDDVVVHEKL YGLTNSSCCL VPRQEDGVSL
VEELLFTSDK VVVDSFFKLK CVVEEKDSVP FEVAHGAKIF EYAATEPRMN
QVENDGMAVF SIVVFEAVFR FYDGFLDMKE LLDVGGGIGT SVSKIVAKYP
LIRGVNFDLP HVISVAPQYP GVEHVAGDMF EEVPKGQNML LKWVLHDWGD
ERCVKLLKNC WNSLPVGGKV LIIEFVLPNE LGNNAESFNA LIPDLLLMAL
NPGGKERTIS EYDDLGKAAG FIKTIPIPIS NGLHVIEFHK
FaOMT (Fragaria ananassa; Q9M602)
(SEQ ID NO: 93)
MGSTGETQMT PTHVSDEEAN LFAMQLASAS VLPMVLKAAI ELDLLEIMAK
AGPGSFLSPS DLASQLPTKN PEAPVMLDRM LRLLASYSIL TCSLRTLPDG
KVERLYCLGP VCKFLTKNED GVSIAALCLM NQDKVLVESW YHLKDAVLDG
GIPFNKAYGM TAFDYHGTDP RENKVENKGM ADHSTITMKK ILETYKGFEG
LKSIVDVGGG TGAVVNMIVS KYPSIKGINF DLPHVIEDAP QYPGVQHVGG
DMFVSVPKGN AIFMKWICHD WSDEHCIKFL KNCYAALPDD GKVILAECIL
PVAPDTSLAT KGVVHMDVIM LAHNPGGKER TEQEFEALAK GSGFQGIRVC
CDAFNTYVIE FLKKI

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an OMT is disclosed and discussed and a number of modifications that can be made to a number of enzymes including the OMT are discussed, each and every combination and permutation of OMT and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a,” “an,” and “the” include plural reference unless the context clearly dictates otherwise. Thus, for example, reference to “a OMT” includes a plurality of such OMTs, reference to “the OMT” is a reference to one or more OMTs and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives. Thus, for example, a list of different OMTs does not indicate that the listed OMTs are obvious one to the other, nor is it an admission of equivalence or obviousness.

Those skilled in the art will recognize, or be able to ascertain using no more than routine experimentation, many equivalents to the specific embodiments of the method and compositions described herein. Such equivalents are intended to be encompassed by the following claims.

We claim:

1. A modified Type 1 O-methyltransferase (OMT), wherein the amino acid corresponding to Phe337 of Sorghum bicolor stilbene OMT (SbSOMT) is substituted with a polar amino acid or wherein the amino acid corresponding to Asn323 of Sorghum bicolor caffeic acid OMT (SbCOMT) is substituted with a hydrophobic amino acid.

2. The modified OMT of claim 1, wherein the amino acid corresponding to Ile144 of SbSOMT is substituted with a polar amino acid or wherein the amino acid corresponding to Asn128 of SbCOMT is substituted with a hydrophobic amino acid.

3. The modified OMT of claim 1, wherein the substituting polar amino acid is independently Asn, Gln, Thr, or Ser.

4. The modified OMT of claim 1, wherein the substituting polar amino acid is Asn.

5. The modified OMT of claim 1, wherein the substituting hydrophobic amino acid is independently Phe, Leu, Trp, Tyr, Ile, Met, Val, or Ala.

6. The modified OMT of claim 1, wherein the substituting hydrophobic amino acid is Phe.

7. The modified OMT of claim 1, wherein the modified OMT is a modified form of Arabidopsis thaliana AtOMT1, Carthamus tinctorius CtCAldOMT, Catharanthus roseus CrCOMT, Hordeum vulgare HvCOMT, Medicago sativa MsCOMT, Medicago sativa MsIOMT, Oryza sativa OsCAldOMT1, Oryza sativa OsNOMT, Panicum virgatum PvCOMT, Pinus sylvestris PsPMT2, Pinus taeda PtAEOMT, Populus tremuloides PtCOMT, Rosa hybrid RhOOMT, Saccharum spontaneum SsSTS, Saccharum spontaneum SsCOMT, Solanum lycopersicum SICOMT, Sorghum bicolor SbCOMT, Sorghum bicolor SbNOMT, Sorghum bicolor SbOMT1, Sorghum bicolor SbOMT3, Sorghum bicolor SbOMT4, Sorghum bicolor SbSOMT, Sorghum bicolor SbSTS1, Sorghum halepense ShOMT1, Sorghum halepense ShSOMT, Triticum aestivum TaCOMT1, Triticum aestivum TaCOMT2, Vitis vinifera VvCOMT, Vitis vinifera VvROMT, Zea mays ZmCOMT, Zea mays ZmNOMT, Medicago truncatula MtIOMT, Medicago truncatula MtIOMT3, Streptomyces carzinostaticus ScNOMT, Loium perenne LpCOMT, Clarkia breweri CbIEOMT, Planctopirus limnophila PlOMT, Thalictrum flavum TfNOMT, Kitagawia praeruptora KpBOMT, Papaver somniferum PsSOMT, or Fragaria ananassa FaOMT.

8. The modified OMT of claim 1, wherein the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

9. The modified OMT of claim 1, wherein the modified OMT has at least 85% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

10. The modified OMT of claim 1, wherein the modified OMT has at least 90% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

11. The modified OMT of claim 1, wherein the modified OMT has at least 95% sequence identity with SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93.

12. The modified OMT of claim 1, wherein the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Phe337 of Sorghum bicolor stilbene OMT (SbSOMT) substituted with a polar amino acid.

13. The modified OMT of claim 1, wherein the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Asn323 of Sorghum bicolor caffeic acid OMT (SbCOMT) substituted with a hydrophobic amino acid.

14. The modified OMT of claim 1, wherein the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Ile144 of SbSOMT substituted with a polar amino acid.

15. The modified OMT of claim 1, wherein the modified OMT is a modified form of SEQ ID NO:1, 2, 32, 33, 5, 6, 7, 8, 9, 10, 11, 12, 13, 14, 15, 16, 17, 18, 19, 20, 21, 22, 23, 24, 25, 26, 27, 28, 29, 34, 35, 82, 83, 84, 85, 86, 87, 88, 89, 90, 91, 92, or 93 with the amino acid corresponding to Asn128 of SbCOMT substituted with a hydrophobic amino acid.

16. The modified OMT of claim 1, wherein the modified OMT is further modified to improve or alter the production of an O-methylated stilbenoid or hydroxycinnamic acid by the modified OMT.

17. A solution comprising the modified OMT of claim 1, wherein the solution is composed to allow production of one or more O-methylated forms of stilbenoid or hydroxycinnamic acid from a substrate for the modified OMT under suitable conditions.

18. A method of producing an O-methylated stilbenoid or hydroxycinnamic acid, the method comprising bringing into contact, in a solution, the modified OMT of claim 1, a substrate for the modified OMT and S-adenosylmethionine; and
incubating under suitable conditions to produce the O-methylated stilbenoid or hydroxycinnamic acid.

19. A plant that expresses the modified OMT of claim 1.

20. A method of producing an O-methylated stilbenoid or hydroxycinnamic acid, the method comprising growing the plant of claim 19 and extracting the O-methylated stilbenoid or hydroxycinnamic acid from the grown plant.


SEQUENCE LISTING (104 sequences)

SEQ ID NO: 1 - protein, 377 aa, Sorghum bicolor
MGSYDSSSSSSNDSSARNEEDESCMFALKLLGGFAVPFTIKAVIELGVMDQLLTAERAMSAEELVAAAVAAQLPRPEVACTMVDRLLRFLASHSVVRCTTEVVVGTDDATTTTCCRRSYAASPVCKWFARNGVEDSVLPLGMMILNKTFLDSWQNITDAVLEGAAPFEKTYGMPMFEYLSTNGPLNTVFHEAMANHSMIITKKLLKFFRGFEGLDVLVDVGGGNGTTLQMIRGQYKNMRGINYDLPHVIAQAAPVEGVEHVGGSMFDNIPRGNAVLLKWILHDWDDKACIKILKNCYTALHVRGKVIVLEYVVPDEPEPTLAAQGAFELDLTMLVTFGSGKERTQREFSELAMEAGFSREFKATYIFANVWALEFTK

SEQ ID NO: 2 - protein, 362 aa, Sorghum bicolor
MGSTAEDVAAVADEEACMYAMQLASSSILPMTLKNALELGLLEVLQKDAGKALAAEEVVARLPVAPTNPAAADMVDRMLRLLASYDVVRCQMEDKDGKYERRYSAAPVGKWLTPNEDGVSMAALALMNQDKVLMESWYYLKDAVLDGGIPFNKAYGMTAFEYHGTDPRFNRVFNEGMKNHSVIITKKLLEFYTGFDESVSTLVDVGGGIGATLHAITSHHSHIRGINFDLPHVISEAPPFPGVQHVGGDMFKSVPAGDAILMKWILHDWSDAHCATLLKNCYDALPEKGGKVIVVECVLPVTTDAVPKAQGVFHVDMIMLAHNPGGRERYEREFRDLAKAAGFSGFKATYIYANAWAIEFIK

SEQ ID NO: 3-4 - skipped (2 entries)

SEQ ID NO: 5 - protein, 363 aa, Arabidopsis thaliana
MGSTAETQLTPVQVTDDEAALFAMQLASASVLPMALKSALELDLLEIMAKNGSPMSPTEIASKLPTKNPEAPVMLDRILRLLTSYSVLTCSNRKLSGDGVERIYGLGPVCKYLTKNEDGVSIAALCLMNQDKVLMESWYHLKDAILDGGIPFNKAYGMSAFEYHGTDPRFNKVFNNGMSNHSTITMKKILETYKGFEGLTSLVDVGGGIGATLKMIVSKYPNLKGINFDLPHVIEDAPSHPGIEHVGGDMFVSVPKGDAIFMKWICHDWSDEHCVKFLKNCYESLPEDGKVILAECILPETPDSSLSTKQVVHVDCIMLAHNPGGKERTEKEFEALAKASGFKGIKVVCDAFGVNLIELLKKL

SEQ ID NO: 6 - protein, 356 aa, Carthamus tinctorius
MAHLEEEEAFLFAMQLASASVLPMVLKSAIDLDLLEIIAKAGPGAHVSPAYLAAQLPKADNPEAAVMLDRICRLLATYSVLTCTLKKLDHGDGDVERLYGLAPVCKFLVKNEDGVSNAPLLLMNQDKILMESWYHLKDAVLDGGIPFNKAYGMSAFEYHGKDERFNKVFNSGMFNHSTMTMKKILDVYPGFNGVKTLVDVGGGTGASLSMILSKHPSIKGINFDLPHVIQDATNYPGIEHVGGDMFESVPKGDAIFMKWICHDWSDAHCLKFLKNCYKALPENGKVIVAECILPETPDSSLATKNVVHIDVIMLAHNPGGKERTEKEFQALAKAAGFKGFHKPCSALNTWLMEFTK

SEQ ID NO: 7 - protein, 363 aa, Catharanthus roseus
MGSANPDNKNSMTKEEEEACLSAMRLASASVLPMVLKSAIELDLLELIKKSGPGAYVSPSELAAQLPTQNPDAPVMLDRILRLLASYSVLNCTLKDLPDGGIERLYSLAPVCKFLTKNEDGVSMAALLLMNQDKVLMESWYHLKDAVLEGGIPFNKAYGMTAFEYHGKDPRFNKVFNQGMSNHSTIIMKKILEIYQGFQGLKTVVDVGGGTGATLNMIVSKYPSIKGINFDLPHVIEDAPSYPGVDHVGGDMFVSVPKGDAIFMKWICHDWSDAHCLKFLKNCHEALPENGKVILAECLLPEAPDSTLSTQNTVHVDVIMLAHNPGGKERTEKEFEALAKGAGFRGFIKVCCAYNSWIMELLK

SEQ ID NO: 8 - protein, 356 aa, Hordeum vulgare
MGSIAAGADEDACMYALQLVSSSILPMTLKNAIELGLLETLMSAGGKFLTPAEVAAKLPSTANPEAPDMVDRMLRLLASYKVVSCRTEESKDGRLSRRYGAAPVCKYLTPNEDGVSMSALALMNQDKVLMESWYYLKDAVLDGGIPFNKAYGMSAFEYHGTDPRFNRVFNEGMKNHSIIITKKLLESYKGFEGLGTLVDVGGGVGATVGAIIARYPAVKGINFDLPHVISEAPAFPGVTHIGGDMFQKVPSGDAILMKWILHDWSDEHCATLLKNCYDALPAHGKVVLVECILPVNPEATPEVQGVFHVDMIMLAHNPGGRERYEREFEALAKGAGFAAMKTTYIYANAWAIEFTK

SEQ ID NO: 9 - protein, 365 aa, Medicago sativa
MGSTGETQITPTHISDEEANLFAMQLASASVLPMILKSALELDLLEIIAKAGPGAQISPIEIASQLPTTNPDAPVMLDRMLRLLACYIILTCSVRTQQDGKVQRLYGLATVAKYLVKNEDGVSISALNLMNQDKVLMESWYHLKDAVLDGGIPFNKAYGMTAFEYHGTDPRFNKVFNKGMSDHSTITMKKILETYTGFEGLKSLVDVGGGTGAVINTIVSKYPTIKGINFDLPHVIEDAPSYPGVEHVGGDMFVSIPKADAVFMKWICHDWSDEHCLKFLKNCYEALPDNGKVIVAECILPVAPDSSLATKGVVHIDVIMLAHNPGGKERTQKEFEDLAKGAGFQGFKVHCNAFNTYIMEFLKKV

SEQ ID NO: 10 - protein, 352 aa, Medicago sativa
MASSINGRKPSEIFKAQALLYKHIYAFIDSMSLKWAVGMNIPNIIHNHGKPISLSNLVSILQVPSSKIGNVRRLMRYLAHNGFFEIITKEEESYALTVASELLVRGSDLCLAPMVECVLDPTLSGSYHELKKWIYEEDLTLFGVTLGSGFWDFLDKNPEYNTSFNDAMASDSKLINLALRDCDFVFDGLESIVDVGGGTGTTAKIICETFPKLKCIVFDRPQVVENLSGSNNLTYVGGDMFTSIPNADAVLLKYILHNWTDKDCLRILKKCKEAVTNDGKRGKVTIIDMVINEKKDENQVTQIKLLMDVNMACLNGKERNEEEWKKLFIEAGFQHYKISPLTGFLSLIEIYP

SEQ ID NO: 11 - protein, 368 aa, Oryza sativa
MGSTAADMAAAADEEACMYALQLASSSILPMTLKNAIELGLLETLQSAAVAGGGGKAALLTPAEVADKLPSKANPAAADMVDRMLRLLASYNVVRCEMEEGADGKLSRRYAAAPVCKWLTPNEDGVSMAALALMNQDKVLMESWYYLKDAVLDGGIPFNKAYGMTAFEYHGTDARFNRVFNEGMKNHSVIITKKLLDLYTGFDAASTVVDVGGGVGATVAAVVSRHPHIRGINYDLPHVISEAPPFPGVEHVGGDMFASVPRGGDAILMKWILHDWSDEHCARLLKNCYDALPEHGKVVVVECVLPESSDATAREQGVFHVDMIMLAHNPGGKERYEREFRELARAAGFTGFKATYIYANAWAIEFTK

SEQ ID NO: 12 - protein, 375 aa, Oryza sativa
MVSPVVHRHAAGGGSGGDDDDQACMYALELLGGSVVSMTLKAAIELGLVDELLAAAGAAVTAEELAARLRLPAAVAAAAAVDRMLRLLASYGVVRCATEAGPDGKARRSYAAAPVCKWLAAGSSSGEGSMAPLGLLNLDKVFMENWYYLKEAVSEGGTAFDKAYGTSLFQYLGQDGNEPSNTLFNQAMASHSVVITNKLLQFFRGFDAGAGVDVLVDVGGGVGATLRMITARHPHLRGVNYDLPHVIAQAPPVEGVEHIGGSMFDHVPSGSAILLKWILHLWGDEECVKILKNCYKALPAKGKVILVEYVLPASPEATLAAQEAFRLDVMMLNRLAGGKERTQQEFTDLAVDAGFSGDCKPTYIFTNVWALEFTK

SEQ ID NO: 13 - protein, 361 aa, Panicum virgatum
MGSTATDVAAAADEEACMYALQLASSSILPMTLKNAIELGLLEVLQKDPAAALAPEEVVAQLPVAPANPDAAAMVDRMLRLLASYDVVRCQMEEGKDGRYSRRYAAAPVCKWLTPNEDGVSMAALALMNQDKVLMESWYYLKDAVLEGGIPFNKAYGMTAFEYHGTDPRFNRVFNEGMKNHSVIITKKLLEFYAGFEGVGTLVDVGGGVGATLHAITSRYPGIRGVNFDLPHVISEAPPFPGVEHVGGDMFKAVPAGDAILMKWILHDWSDAHCAAILKNCYDALPAGGKVIAVECILPVNPEATPKAQGVFHVDMIMLAHNPGGKERYEREFEELAKGAGFTGFKATYIYANAWAIEFTK

SEQ ID NO: 14 - protein, 361 aa, Pinus sylvestris
MNMQSVKDEEALRASALGLAFSLETPFLLKCAIRLKIPDIISKAGPDVSLSVHQIAAQLPSEDPDMGALSRILTYLSTMGILQAIVPPEGVNAPMNIRYGLTNLTKTYFTSEDISSRSLVPFVLLQTHPLYVTAWDNIHERVLHGGDNFKNSSGNGKDFWNFAAGEPEFNAIFNAGMVSVTKATITYVLAVYDGFKDINTLVDVGGGRGEALSLITEAHPHIRAINFDLPQVIATAPTIPGVQHMSGNLFESAPSADAIFMKNFLHSWNDEDCIKLLNNCHQALPEKGKLILSEAILDLTEGSDMIGSANVLDAVMLNCLPGGGERTRKQWNDLLQAAGFSISKIVGRNGTLTKVIEAIKS

SEQ ID NO: 15 - protein, 381 aa, Pinus taeda
MDSNMNGLAKSNGCEISRDGFFESEEEELQGQAEAWKCTFAFAESLAVKCVVLLGIPDMIAREGPRATLSLCEIVANLPTESPDAACLFRIMRFLVAKGIFPASKSARRRAFETRYGLTPASKWLVKGRELSMAPMLLMQNDETTLAPWHHFNECVLEGGVAFQKANGAEIWSYASDHPDFNNLFNNAMACNARIVMKAILSKYQGFHSLNSLVDVGGGTGTAVAEIVRAYPFIRGINYDLPHVVATASSLSGVQHVGGDMFETVPTADAIFMKWIMHDWNDEDCIKILKNCRKAIPDTGKVIIVDVVLDADQGDNTDKKRKKAVDPIVGTVFDLVMVAHSSGGKERTEKEWKRILLEGGFSRYNIIEIPALQSVIEAFPR

SEQ ID NO: 16 - protein, 365 aa, Populus tremuloides
MGSTGETQMTPTQVSDEEAHLFAMQLASASVLPMILKTAIELDLLEIMAKAGPGAFLSTSEIASHLPTKNPDAPVMLDRILRLLASYSILTCSLKDLPDGKVERLYGLAPVCKFLTKNEDGVSVSPLCLMNQDKVLMESWYYLKDAILDGGIPFNKAYGMTAFEYHGTDPRFNKVFNKGMSDHSTITMKKILETYKGFEGLTSLVDVGGGTGAVVNTIVSKYPSIKGINFDLPHVIEDAPSYPGVEHVGGDMFVSVPKADAVFMKWICHDWSDAHCLKFLKNCYDALPENGKVILVECILPVAPDTSLATKGVVHVDVIMLAHNPGGKERTEKEFEGLAKGAGFQGFEVMCCAFNTHVIEFRKKA

SEQ ID NO: 17 - protein, 367 aa, Rosa sp.
MERLNSFRHLNQKWSNGEHSNELLHAQAHIWNHIFSFINSMSLKSAIQLGIPDIINKHGYPMTLSELTSALPIHPTKSHSVYRLMRILVHSGFFAKKKLSKTDEEGYTLTDASQLLLKDHPLSLTPYLTAMLDPVLTNPWNYLSTWFQNDDPTPFDTAHGMTFWDYGNHQPSIAHLFNDAMASDARLVTSVIINDCKGVFEGLESLVDVGGGTGTLAKAIADAFPHIECTVLDLPHVVADLQGSKNLKYTGGDMFEAVPPADTVLLKWILHDWSDEECIKILERSRVAITGKEKKGKVIIIDMMMENQKGDEESIETQLFFDMLMMALVGGKERNEKEWAKLFTDAGFSDYKITPISGLRSLIEVYP

SEQ ID NO: 18 - protein, 357 aa, Solanum lycopersicum
MGSTSLTQTEDEAFLFAMQLASASVLPMVLKSAVELELLELMAKAGPGASISPAELASQLPCKNPDAPVMLDRMLRLLAAYSVLNCTLRTLPDGRVERLYSLAPVCKFLTKNADGVSVAPLLLMNQDKVLMQSWYHLKDAVLDGGIPFNKAYGMTAFEYHGTDPRFNKVFNRGMSDHTTLSIKKILEDYKGFEGLNSIVDVGGGTGATVSMIVSKYPSIKGINFDLPHVIEDAPAYPGVEHIGGDMFVSVPKADAIFMKWICHDWSDEHCLKFLKNCYEAVPANGKVIIAECLLPEVPDTSSSTKNTVHVDVIMLAHNPGGKERTEKEFEALAKGAGFNGFTKASCAYNTWIMEFTK

SEQ ID NO: 19 - protein, 364 aa, Sorghum bicolor
MACTTAASLQHDKANDDEACMYAQELLFSFIVPMTLKAVIELGLIDYLLAADGRSVTPEELAAEWPQSAEAAAAVDRMMRLLASHSVVRCTTEVGPDGKARRSYAAAPVCKWLATRNAGGQGSLAPMGLMNLNKAFMETWYFMKEAVTEGATPTEKAYGMPLFEHLGSDEASNTLFNQAMAGHSEMIIKKLLEVYRGFEGVDVLVDVGGGTGSTLRMVTAQYKHLRGVNYDLPHVIAQAPPVQGVEHVGGSMFEYIPSGNAILLKWILHLWRDDECVKILKNCHRALPANGKVIVVEYVLPASPEPTQVAQVSLLLDVAMLNRLRGAKERTEQEFAQLAAEAGFSGGCRATYVFASAWALEFTK

SEQ ID NO: 20 - protein, 376 aa, Sorghum bicolor
MASYTSTSGQFAVGKVAAANQDDETCMHALKLLGGLAVPFTIKAVIELGIMDLLLAADRAMTAEALTAALLCPAPAPAAAAAMVDRMLRFLASHGVVRCATESEELGSDDGKSCRRYAAAPVCKWFARGGGVESVVPMGFWMTSTTNMETWHNIKDGVLAGETPFDKAYGMPVFEYLGANGTMNTLFNEAMASHSMIITKRLLEVFRGFENYSVLVDVGGGNGTTMQMIRSQYENISGINYDLPHVIAQASPIEGVEHVAGNMFDNIPRGDAIILKWILHNWGDKECVKILKNCYTALPVNGTVIILEYILPETPEETLASQLAFDFDLGMMLFFGASGKERTEKELLELAREAGFSGDYTATYIFANVWAHEFTK

SEQ ID NO: 21 - protein, 374 aa, Sorghum bicolor
MVLISEDSRELLQAHVELWNQTYSFMKSVALAVALDLHIADAIHRRGGAATLSQILGEIGVRPCKLPGLHRIMRVLTVSGTFTIVQPSAETMSSESDGREPVYKLTTASSLLVSSESSATASLSPMLNHVLSPFRDSPLSMGLTAWFRHDEDEQAPGMCPFTLMYGTTLWEVCRRDDAINALFNNAMAADSNFLMQILLKEFSEVFLGIDSLVDVAGGVGGATMAIAAAFPCLKCTVLDLPHVVAKAPSSSIGNVQFVGGDMFESIPPANVVLLKWILHDWSNDECIKILKNCKQAIPSRDAGGKIIIIDVVVGSDSSDTKLLETQVIYDLHLMKIGGVERDEQEWKKIFLEAGFKDYKIMPILGLRSIIELYP

SEQ ID NO: 22 - protein, 374 aa, Sorghum bicolor
MGSYTTRVHAVEKAANNQDDETCMHALMLLGGLAVPCTIKAVIELGIMDLLLAADRAMTAEELTARLPCPAAATAAAMVDRMLRFLASHGVVRCAAATKESSELGSDGGKSCRRYAAAPVCRWFTRSGGVESVVPMGLWMTGKTVLETWYNIKEAVLEGETPFDRAYGQPFFEYLGENGTVNTLFDEAMANHSTIITKRLVEVFRGFENYSVLVDVGGNKGTTLQMIRSQYENISGINYDLPRVIAQAPPIEGVEHVGGNMFDNVPRGDAIILKWILHDWGDKDCVKILKNCYAALPVNGTMIILEYILPETPEETLTSQLAFNFDFGMMLMYGAKGKERTEKELSELAREAGFSGDCTATYIFASIWALEFTK

SEQ ID NO: 23 - protein, 398 aa, Sorghum bicolor
MTTGKVTLEAVRKAQRAEGPATVLAIGTATPANCVYQADYPDYYFRVTKSEHLTDLKEKFKRICHKSMIRKRYMHLTEDILEENPNMSSYWAPSLDARQDILIQEIPKLGAEAAEKALKEWGQPRSRITHLVFCTTSGVDMPGADYQLIKLLGLCPSVNRAMMYHQGCFAGGMVLRLAKDLAENNRGARVLIVCSEITVVTFRGPSESHLDSLVGQALFGDGAAAVIVGADPSEPAERPLFHLVSASQTILPDSEGAIEGHLREVGLTFHLQDRVPQLISMNIERLLEDAFAPLGISDWNSIFWVAHPGGPAILNMVEAKVGLDKARMCATRHILAEYGNMSSVCVLFILDEMRNRSAKDGHTTTGEGMEWGVLFGFGPGLTVETIVLHSVPITTVAA

SEQ ID NO: 24 - protein, 360 aa, Triticum aestivum
MGSTAADMAASADEEACMYALQLVSSSILPMTLKNAIELGLLETLVAAGGKLLTPAEVAAKLPSTANPAAADMVDRMLRLLASYNVVSCTMEEGKDGRLSRRYRAAPVCKFLTPNEDGVSMAALALMNQDKVLMESWYYLKDAVLDGGIPFNKAYGMSAFEYHGTDPRFNRVFNEGMKNHSIIITKKLLEVYKGFEGLGTIVDVGGGVGATVGAITAAYPAIKGINFDLPHVISEAQPFPGVTHVGGDMFQKVPSGDAILMKWILHDWSDEHCATLLKNCYDALPAHGKVVLVECILPVNPEATPKAQGVFHVDMIMLAHNPGGRERYEREFEALAKGAGFKAIKTTYIYANAFAIEFTK

SEQ ID NO: 25 - protein, 356 aa, Triticum aestivum
MGSIAAGADEDACMYALQLVSSSILPMTLKNAIELGLLETLMAAGGKFLTPAEVAAKLPSAANPEAPDMVDRMLRLLASYNVVSCRTEDGKDGRLSRRYGAAPVCKYLTPNEDGVSMSALALMNQDKVLMESWYYLKDAVLDGGIPFNKAYGMSAFEYHGTDPRFNRVFNEGMKNHSIIITKKLLESYKGFEGLGTLVDVGGGVGATVAAITAHYPTIKGINFDLPHVISEAPPFPGVTHVGGDMFQKVPSGDAILMKWILHDWSDEHCATLLKNCYDALPAHGKVVLVECILPVNPEATPKAQGVFHVDMIMLAHNPGGRERYEREFEALAKGAGFAAMKTTYIYANAWAIEFTK

SEQ ID NO: 26 - protein, 386 aa, Vitis vinifera
MESTLAFNSGSNSMNQSFSSSAEFNSPVPETIPKSEEDTFVFATLLTSASVLPMALKSALELDLLEIIAKAGPGAFVSTSEIAAKITKRNPKAPVMLDRILRLLATYDVVKCSLRDSPDGGVERLYGLGPVCKYFTTNEDGVSVAPLLLMNQDKVPMQSKRYHLKDAVLDGGIPFNKAYGMTDFEYHGTEPRFNKVFNNGVSGHPTITMKKILEAYKGFEGLTSIVDVGGGTGATLNMIISKYPTIKGINFDLPHVIDDAPSYPGVEHVGGDMFVSVPKGDAIFMKWMCYEWDDAHCLKFLENCYQALPDNGKVIVAECILPVVPDTSLATKSAVHIDVIMLAYNTGGKARTEKEFEALAKGAGFQGFKVVCCAFNSWIMEFCKTA

SEQ ID NO: 27 - protein, 357 aa, Vitis vinifera
MDLANGVISAELLHAQAHVWNHIFNFIKSMSLKCAIQLGIPDIIHNHGKPMTLPELVAKLPVHPKRSQCVYRLMRILVHSGFLAAQRVQQGKEEEGYVLTDASRLLLMDDSLSIRPLVLAMLDPILTKPWHYLSAWFQNDDPTPFHTAHERSFWDYAGHEPQLNNSFNEAMASDARLLTSVLLKEGQGVFAGLNSLVDVGGGTGKVAKAIANAFPHLNCTVLDLPHVVAGLQGSKNLNYFAGDMFEAIPPADAILLKWILHDWSDEECVKILKRCREAIPSKENGGKVIIIDMIMMKNQGDYKSTETQLFFDMTMMIFAPGRERDENEWEKLFLDAGFSHYKITPILGLRSLIEVYP

SEQ ID NO: 28 - protein, 364 aa, Zea mays
MGSTAGDVAAVVDEEACMYAMQLASSSILPMTLKNAIELGLLEVLQKEAGGGKAALAPEEVVARMPAAPGDPAAAAAMVDRMLRLLASYDVVRCQMEDRDGRYERRYSAAPVCKWLTPNEDGVSMAALALMNQDKVLMESWYYLKDAVLDGGIPFNKAYGMTAFEYHGTDSRFNRVFNEGMKNHSVIITKKLLDFYTGFEGVSTLVDVGGGVGATLHAITSRHPHISGVNFDLPHVISEAPPFPGVRHVGGDMFASVPAGDAILMKWILHDWSDAHCATLLKNCYDALPENGKVIVVECVLPVNTEATPKAQGVFHVDMIMLAHNPGGKERYEREFRELAKGAGFSGFKATYIYANAWAIEFIK

SEQ ID NO: 29 - protein, 365 aa, Zea mays
MACTTAASLQHDRANDDEACMYAQELLSCFVVPMTLKAVIELGLIDDLLAADGRFVTPEELAARWARPAEAAAAVDRMLRFLASHSVVRCTTEAAGPDGRARRSYAAAPVCKWLIARNGTGQGSWAPIGLMNLNKGFMETWYYMKDAVAEGATPTEKAYGMPLFEHLGSDEALNTLFNQAMAGHSEIVISKLLEVYRGFEGVDVLVDVGGGTGSTLRMVTAQYKHLRGVNYDLPHVIAQAPPVQGVEHAGGSMFEYIPSGNAILLKWILHLWRDEECIKILKNCHRALPANGKVIVVECVLPASPEPTQVAQGALLLDVVMLNRLRGAKERTEREFTELAAEAGFSGGCRATYVFTGAWALEFTK

SEQ ID NO: 30 - DNA, 1125 bp, Sorghum halepense
  source 1..1125: mol_type = genomic DNA
atgggcagctatactaccagagtccacgccgtagagaaagcagctaacaaccaagatgatgagacgtgcatgcatgctttgatgctccttggcggattggccgtaccttgtaccatcaaggcggtcatcgagctcggcatcatggaccttctcctcgccgcggaccgcgccatgaccgcggaggagctcacggcaaggttgccatgtcctgctgctgctactgctgctgctatggtcgaccgcatgctccgcttcctagcttcgcacggcgtggtcaggtgcgccgccgccaccaaggagtcgtcggagctgggctccgatggtggcaagagctgccggcgctacgcggcggcgccggtgtgcaggtggtttacaaggagcggcggcgtggagtcggtggttccaatggggttgtggatgaccggcaagaccgtcttggagacctggtacaacataaaagaagcagtgttagagggagaaacaccatttgacagagcatacggccagccattttttgagtaccttggtgaaaacgggacagtgaacacgttgttcgatgaggcaatggcgaaccattcgacaatcataacgaagaggctggtcgaggtcttccgtggctttgagaattacagtgtgctcgtcgatgtcggcggcaacaaaggcaccacgttgcaaatgattagaagtcagtatgagaatattagtggcatcaactacgaccttcctcgtgtaattgcgcaggcacctccaattgaaggtgtggagcatgttggtggcaacatgtttgataatgttccacgtggagatgcaattatcctaaagtggattcttcacgattggggcgacaaggactgcgtcaagatcctaaagaattgctatgcagctctcccggtgaacgggacgatgatcattctggagtacatcctcccagagacaccagaagaaacactaacatcgcaactagcgttcaacttcgatttcgggatgatgctcatgtacggcgccaaaggtaaggagagaacagagaaagagttgtcggagctcgccagagaggccggcttctctggagactgcacggctacatacatcttcgccagcatctgggcccttgaattcacaaagtaa

SEQ ID NO: 31 - DNA, 1188 bp, Sorghum halepense
  source 1..1188: mol_type = genomic DNA
tcgtcctctatctgtggccaccaactgctgtgctatttgagtacacagatacagatgggcagctacgacagcagcagtagtagtagtaatgactcatcagcacggaacgaggaggacgagtcgtgcatgtttgctttgaagctcctcggcgggttcgccgtacctttcaccatcaaggcggtgattgagctgggcgtcatggaccagctcctgactgccgaacgcgcgatgagcgcggaggagctggtggcagcagcagtggcagcacagctgccacggcccgaggtagcctgcaccatggtggaccgcctgctccgtttccttgcctcgcacagcgtagtccggtgcacgaccgaggtggtggtgggcacggacgacgccaccaccaccacctgctgccgccggagctacgccgcgtcacccgtctgcaagtggttcgccaggaacggcgtcgaggattcggtgcttccgcttgggatgatgatcctaaacaagacattcctggacagctggcaaaacattacggatgcagtgttggaaggagcagcaccatttgagaaaacctacgggatgccaatgttcgagtacctaagtacaaacggaccattgaacacggtgttccacgaggcaatggcaaatcattcgatgattataaccaagaaactgctcaagttcttccgcggcttcgaaggccttgatgtgctggtcgacgtaggcggcggcaacggcaccacgttgcaaatgattagaggtcaatataagaatatgagaggcataaactacgaccttcctcatgtcattgcgcaggctgcaccagttgaaggtgtggaacatgtaggtggcagcatgttcgataatattccacgcggaaatgcagttctgctcaagtggattcttcatgattgggacgacaaggcgtgcatcaagatcctaaagaattgctatacagctctccatgtgagaggcaaggtgatcgttctggagtacgttgttccggatgaaccagaacctactcttgcagctcagggtgccttcgaactggacctcaccatgctggtcacgtttggcagtggtaaagagaggacacagagggagttctccgagctcgccatggaggccggcttctctagagagtttaaggctacgtatatctttgccaacgtctgggcccttgagttcacaaagtaa

SEQ ID NO: 32 - protein, 362 aa, Saccharum spontaneum
MGSTAEDVAAVADEEACMYAMQLASASILPMTLKNALELGLLEVLQAEAPAGKALAPEEVVARLPVAPTNPDAADMVDRMLRLLASYDVVKCQMEDKDGKYERRYSAAPVGKWLTPNEDGVSMAALTLMNQDKVLMESRYYLKDAVLDGGIPFNKAYGMTAFEYHGTDPRFNRVFNEGMKNHSVIITKKLLEFYTGFEGVSTLVDVGGGIGATLHAITSHHPQIKGINFDLPHVISEAPPFPGVQHVGGDMFKSVPAGDAILMKWILHDWSDAHCATLLKNCYDALPENGKVIVVECVLPVNTEAVPKAQGVFHVDMIMLAHNPGGRERYEREFHDLAKGAGFSGFKATYIYANAWAIEFIK

SEQ ID NO: 33 - protein, 394 aa, Saccharum spontaneum
MTGKVTLGAVRKAQRAEGSAAVLAIGTATPANCVYQADYPDYYFRVTKSEHLTDLKEKFKRICHKSMITKRYMHLTEGFLQENPNMSSYSAPSLDARQDILIEEVPKLGAAAAEKALKEWGQPRSQITHLVFCTTSGVDMPGADYQLIKLLGLSLSVNRAMMYHQGCFAGGMVLRLAKDLAENNRGARVLIVCSEITAVTFRGPSESHLDSLVGQALFGDGAAAVIVGADPSAAEWPLFQLVSASQTILPDSEGAIEGHLREVGLTFHLQDRVPQLISTNIERLLEDAFTPLGISDWNSIFWVAHPGGPAILNMVEAKAGLDKARLCATRHILAEYGNMSSACVLFILDEMRNKSAEDGHTTTGEGMEWGVLFGFGPGLTVETIVLQSVPIITV

SEQ ID NO: 34 - protein, 374 aa, Sorghum halepense; identical to SEQ ID NO: 22

SEQ ID NO: 35 - protein, 377 aa, Sorghum halepense; identical to SEQ ID NO: 1

SEQ ID NO: 36 - DNA, 21 bp, synthetic construct
  source 1..21: mol_type = other DNA
caactttgtcacccgcgatga

SEQ ID NO: 37 - DNA, 21 bp, synthetic construct
  source 1..21: mol_type = other DNA
tccagaaaccttagcagccca

SEQ ID NO: 38 - DNA, 19 bp, synthetic construct
  source 1..19: mol_type = other DNA
tgctacggtgttggccatt

SEQ ID NO: 39 - DNA, 23 bp, synthetic construct
  source 1..23: mol_type = other DNA
atcgacttgtggcatatcctctt

SEQ ID NO: 40 - DNA, 24 bp, synthetic construct
  source 1..24: mol_type = other DNA
cctaaagtggattcttcacgattg

SEQ ID NO: 41 - DNA, 23 bp, synthetic construct
  source 1..23: mol_type = other DNA
gctagttgcgatgttagtgtttc

SEQ ID NO: 42 - DNA, 21 bp, synthetic construct
  source 1..21: mol_type = other DNA
aggtggcagcatgttcgataa

SEQ ID NO: 43 - DNA, 20 bp, synthetic construct
  source 1..20: mol_type = other DNA
acgatcaccttgcctctcac

SEQ ID NO: 44 - DNA, 21 bp, synthetic construct
  source 1..21: mol_type = other DNA
ttggtttccactgacttcgtt

SEQ ID NO: 45 - DNA, 19 bp, synthetic construct
  source 1..19: mol_type = other DNA
ctgtagccccactcgttgt

SEQ ID NO: 46 - DNA, 20 bp, synthetic construct
  source 1..20: mol_type = other DNA
agagcgaacaccttaccgac

SEQ ID NO: 47 - DNA, 20 bp, synthetic construct
  source 1..20: mol_type = other DNA
gccgagtacgagctcatgtt

SEQ ID NO: 48 - DNA, 20 bp, synthetic construct
  source 1..20: mol_type = other DNA
gaggacaaggacggcaagta

SEQ ID NO: 49 - DNA, 20 bp, synthetic construct
  source 1..20: mol_type = other DNA
accgcgtccttgaggtagta

SEQ ID NO: 50 - DNA, 29 bp, synthetic construct
  source 1..29: mol_type = other DNA
cgcggatccatgggcagctatactaccag

SEQ ID NO: 51 - DNA, 29 bp, synthetic construct
  source 1..29: mol_type = other DNA
cccaagcttctttgtgaattcaagggccc

SEQ ID NO: 52 - DNA, 42 bp, synthetic construct
  source 1..42: mol_type = other DNA
tgagaacctgtacttccaaggcagctacgacagcagcagtag

SEQ ID NO: 53 - DNA, 45 bp, synthetic construct
  source 1..45: mol_type = other DNA
agccggatctcactcgagttactttgtgaactcaagggcccagac

SEQ ID NO: 54 - DNA, 37 bp, synthetic construct
  source 1..37: mol_type = other DNA
tgagaacctgtacttccaagggtcgacggcggaggac

SEQ ID NO: 55 - DNA, 43 bp, synthetic construct
  source 1..43: mol_type = other DNA
agccggatctcactcgagttacttgatgaactcgatggcccag

SEQ ID NO: 56 - DNA, 45 bp, synthetic construct
  source 1..45: mol_type = other DNA
cttctgcaggaattcgatatcatgactgggaaggtaacattgggg

SEQ ID NO: 57 - DNA, 44 bp, synthetic construct
  source 1..44: mol_type = other DNA
gagagatctgtcgacgatatcctacactgtgatgatgggaacgc

SEQ ID NO: 58 - DNA, 36 bp, synthetic construct
  source 1..36: mol_type = other DNA
cttctgcaggaattcgatatcatgggctcgaccgcc

SEQ ID NO: 59 - DNA, 46 bp, synthetic construct
  source 1..46: mol_type = other DNA
gagagatctgtcgacgatatcttacttgatgaactcgatggcccag

SEQ ID NO: 60 - DNA, 48 bp, synthetic construct
  source 1..48: mol_type = other DNA
gtgcttccgcttgggatgatgaacctaaacaagacattcctggacagc

SEQ ID NO: 61 - DNA, 21 bp, synthetic construct
  source 1..21: mol_type = other DNA
catcatcccaagcggaagcac

SEQ ID NO: 62 - DNA, 45 bp, synthetic construct
  source 1..45: mol_type = other DNA
cctcaccatgctggtcacgaacggcagtggtaaagagaggacaca

SEQ ID NO: 63 - DNA, 19 bp, synthetic construct
  source 1..19: mol_type = other DNA
cgtgaccagcatggtgagg

SEQ ID NO: 64 - DNA, 43 bp, synthetic construct
  source 1..43: mol_type = other DNA
ttctgctcaagtggattcttgctgattgggacgacaaggcgtg

SEQ ID NO: 65 - DNA, 25 bp, synthetic construct
  source 1..25: mol_type = other DNA
aagaatccacttgagcagaactgca

SEQ ID NO: 66 - DNA, 20 bp, synthetic construct
  source 1..20: mol_type = other DNA
gattgggacgacaaggcgtg

SEQ ID NO: 67 - DNA, 24 bp, synthetic construct
  source 1..24: mol_type = other DNA
gttaagaatccacttgagcagaac

SEQ ID NO: 68 - DNA, 16 bp, synthetic construct
  source 1..16: mol_type = other DNA
gcgtgggacgacaagg

SEQ ID NO: 69 - DNA, 24 bp, synthetic construct
  source 1..24: mol_type = other DNA
atgaagaatccacttgagcagaac

SEQ ID NO: 70 - DNA, 19 bp, synthetic construct
  source 1..19: mol_type = other DNA
gcgtacgttgttccggatg

SEQ ID NO: 71 - DNA, 20 bp, synthetic construct
  source 1..20: mol_type = other DNA
cagaacgatcaccttgcctc

SEQ ID NO: 72 - DNA, 21 bp, synthetic construct
  source 1..21: mol_type = other DNA
gcgaggacacagagggagttc

SEQ ID NO: 73 - DNA, 21 bp, synthetic construct
  source 1..21: mol_type = other DNA
tttaccactgccaaacgtgac

SEQ ID NO: 74 - DNA, 20 bp, synthetic construct
  source 1..20: mol_type = other DNA
tgactcatcagcacggaacg

SEQ ID NO: 75 - DNA, 20 bp, synthetic construct
  source 1..20: mol_type = other DNA
caccctcctagtagccgcaa

SEQ ID NO: 76 - DNA, 22 bp, synthetic construct
  source 1..22: mol_type = other DNA
ggcaaaacattacggatgcagt

SEQ ID NO: 77 - DNA, 21 bp, synthetic construct
  source 1..21: mol_type = other DNA
tgacttcacggaggctcatag

SEQ ID NO: 78 - DNA, 31 bp, synthetic construct
  source 1..31: mol_type = other DNA
atcggaccggtatgacgactgggaaggtaac

SEQ ID NO: 79 - DNA, 31 bp, synthetic construct
  source 1..31: mol_type = other DNA
cgatcctcgagtcatgcagccactgtggtga

SEQ ID NO: 80 - DNA, 33 bp, synthetic construct
  source 1..33: mol_type = other DNA
tgcccaaattcgcgaatgggcagctacgacagc

SEQ ID NO: 81 - DNA, 67 bp, synthetic construct
  source 1..67: mol_type = other DNA
tggtgatggtgatgcatattatttttcaaattgaggatgagaccactttgtgaactcaagggcccag

SEQ ID NO: 82 - protein, 372 aa, Medicago sativa
MGNSYITKEDNQISATSEQTEDSACLSAMVLTTNLVYPAVLNAAIDLNLFEIIAKATPPGAFMSPSEIASKLPASTQHSDLPNRLDRMLRLLASYSVLTSTTRTIEDGGAERVYGLSMVGKYLVPDESRGYLASFTTFLCYPALLQVWMNFKEAVVDEDIDLFKNVHGVTKYEFMGKDKKMNQIFNKSMVDVCATEMKRMLEIYTGFEGISTLVDVGGGSGRNLELIISKYPLIKGINFDLPQVIENAPPLSGIEHVGGDMFASVPQGDAMILKAVCHNWSDEKCIEFLSNCHKALSPNGKVIIVEFILPEEPNTSEESKLVSTLDNLMFITVGGRERTEKQYEKLSKLSGFSKFQVACRAFNSLGVMEFYK

SEQ ID NO: 83 - protein, 365 aa, Medicago sativa; identical to SEQ ID NO: 9

SEQ ID NO: 84 - protein, 364 aa, Medicago truncatula
MAFSTNGSEESELYHAQIHLYKHVYNFVSSMALKSAMELGIADAIHNHGKPMTLSELASSLKLHPSKVNILHRFLRLLTHNGFFAKTIVKGKEGDEEEEIAYSLTPPSKLLISGKPTCLSSIVKGALHPSSLDMWSSSKKWFNEDKEQTLFECATGESFWDFLNKDSESSTLSMFQDAMASDSRMFKLVLQENKRVFEGLESLVDVGGGTGGVTKLIHEIFPHLKCTVFDQPQVVGNLTGNENLNFVGGDMFKSIPSADAVLLKWVLHDWNDEQSLKILKNSKEAISHKGKDGKVIIIDISIDETSDDRGLTELQLDYDLVMLTMFLGKERTKQEWEKLIYDAGFSSYKITPISGFKSLIEVYP

SEQ ID NO: 85 - protein, 357 aa, Medicago truncatula
MASSINNRKPSEIFKAQALLYKNMYAFVDSMSLKWSIEMNIPNIIHNHGKPITLSNLVSILQIPSTKVDNVQRLMRYLAHNGFFEIITNQELENEEEAYALTVASELLVKGTELCLAPMVECVLDPTLSTSFHNLKKWVYEEDLTLFAVNLGCDLWEFLNKNPEYNTLYNDALASDSKMINLAMKDCNLVFEGLESIVDVGGGNGTTGKIICETFPKLTCVVFDRPKVVENLCGSNNLTYVGGDMFISVPKADAVLLKAVLHDWTDKDCIKILKKCKEAVTSDGKRGKVIVIDMVINEKKDENQLTQIKLLMNVTISCVNGKERNEEEWKKLFIEAGFQDYKISPFTGLMSLIEIYP

SEQ ID NO: 86 - protein, 332 aa, Streptomyces sp.
MGKRAAHIGLRALADLATPMAVRVAATLRVADHIAAGHRTAAEIASAAGAHADSLDRLLRHLVAVGLFTRDGQGVYGLTEFGEQLRDDHAAGKRKWLDMNSAVGRGDLGFVELAHSIRTGQPAYPVRYGTSFWEDLGSDPVLSASFDTLMSHHLELDYTGIAAKYDWAALGHVVDVGGGSGGLLSALLTAHEDLSGTVLDLQGPASAAHRRFLDTGLSGRAQVVVGSFFDPLPAGAGGYVLSAVLHDWDDLSAVAILRRCAEAAGSGGVVLVIEAVAGDEHAGTGMDLRMLTYFGGKERSLAELGELAAQAGLAVRAAHPISYVSIVEMTAL

SEQ ID NO: 87 - protein, 360 aa, synthetic construct
MGSTAADMAASADEDACMFALQLASSSVLPMTLKNAIELGLLEILVAAGGKSLTPTEVAAKLPSAANPEAPDMVDRILRLLASYNVVTCLVEEGKDGRLSRSYGAAPVCKFLTPNEDGVSMAALALMNQDKVLMESWYYLKDAVLDGGIPFNKAYGMSAFEYHGTDPRFNRVFNEGMKNHSIIITKKLLELYHGFEGLGTLVDVGGGVGATVAAIAAHYPTIKGVNFDLPHVISEAPQFPGVTHVGGDMFKEVPSGDTILMKWILHDWSDQHCATLLKNCYDALPAHGKVVLVQCILPVNPEANPSSQGVFHVDMIMLAHNPGGRERYEREFQALARGAGFTGVKSTYIYANAWAIEFTK

SEQ ID NO: 88 - protein, 368 aa, Clarkia breweri
MGSTGNAEIQIIPTHSSDEEANLFAMQLASAAVLPMALKAAIELDVLEIMAKSVPPSGYISPAEIAAQLPTTNPEAPVMLDRVLRLLASYSVVTYTLRELPSGKVERLYGLAPVCKFLTKNEDGVSLAPFLLTATDKVLLEPWFYLKDAILEGGIPFNKAYGMNEFDYHGTDHRFNKVFNKGMSSNSTITMKKILEMYNGFEGLTTIVDVGGGTGAVASMIVAKYPSINAINFDLPHVIQDAPAFSGVEHLGGDMFDGVPKGDAIFIKWICHDWSDEHCLKLLKNCYAALPDHGKVIVAEYILPPSPDPSIATKVVIHTDALMLAYNPGGKERTEKEFQALAMASGFRGFKVASCAFNTYVMEFLKTA

SEQ ID NO: 89 - protein, 361 aa, unidentified
  source 1..361: note = Planctopirus limnophila
MAVKDALRFPPTDVTPIFDLFRGNFATELLAASVAHLHVFDILNESPLSLDELQRRLVLSERATQVLVTGLCAMQLLTKRLAGEIDLTPLARNHLVTTSPFSVGGYISLAAQSAGTLALVERLKSDRPEGAESEQGAAFIFREGSESAMDREDSARFLTLSLAGRAWNVAPRFADVLPAGQPGKILKDNSGSSGRVLLDVAGGSGIYTMAVLQKYPTWRGIIFDRPEVLKIAAELAEQTGVRDRLELHAGDMWVDPFPPADDILLSNVLHDWDRPQCARLVAKATSGLPEGGRLLIHDVLLNSDLTGPLEIALYSLALFSLTEGRAYSLEEYRGWIAGADLKYVDCIPTSAHGHLILSEKV

SEQ ID NO: 90 - protein, 350 aa, Thalictrum sp.
MEMINKENLSSQAKLWNFIYGFADSLVLKSAVQLDLANIIHNHGSPMTLSELSLHLPSQPVNQDALYRVLRYLVHMKLFTKSSIDGELRYGLAPPAKFLVKGWDKCMLGAILTITDKDFMAPWHYLKEGILNDGSTSTAFEKALGTNIWDYMAEHPEKNQLFNEGMANDTRLIMSALVKECSSMFDGITTIVDVGGGTGTAVRNIAKAFPHIKCTVYDLPHVIADSPGYTEINSIQGDMFKYIPNADAIMMKCILHDWDDKECIEILKRCKDAVPRDGGKVIIIDIILDVKSEHPYTKMRLTLDLDMMLNTGGKERTEEEWKKLIHDAGYKGYKITHISAVQSVIEAYPY

SEQ ID NO: 91 - protein, 359 aa, unidentified
  source 1..359: note = Kitagawia praeruptora
MAGMKTSPSQDEEACVLAIQLATSTVLPMILKSAIELDILNTISKAGPGNYLSPSDLASKLLMSNPHAPIMLERILRVLATYKVLGCKPSELSDGEVEWLYCWTPVCKFLSNNEDGASIAPLLLVHQDQVPMKSWYHLTDAILDGGTAFNKAYGMNIFDYASQDPQFNKVFNRSMAGHSTITMKKILETYNGFEGLKSIVDVGGGSGATLNMIISKYPTIKGINFDLPHVVGDSPIHPGVEHVGGDMFASVPKGDAIFLKWIFHSWSDEDCLRILKNCYEALADNKKVIVAEFIIPEVPGGSDDATKSVVHLDAVMLAYVPGGKERTEKEFEALATSAGFKSFRKVCCAFNTWIMEFSK

SEQ ID NO: 92 - protein, 390 aa, Papaver somniferum
MATNGEIFNTYGHNRQTATVTKITASNESSNGVCYLSETANLGKLICIPMALRAAMELNVFQLISKFGTDAKVSASEIASKMPNAKNNPEAAMYLDRILRLLGASSILSVSTTKKSINRGGDDVVVHEKLYGLTNSSCCLVPRQEDGVSLVEELLFTSDKVVVDSFFKLKCVVEEKDSVPFEVAHGAKIFEYAATEPRMNQVFNDGMAVFSIVVFEAVFRFYDGFLDMKELLDVGGGIGTSVSKIVAKYPLIRGVNFDLPHVISVAPQYPGVEHVAGDMFEEVPKGQNMLLKWVLHDWGDERCVKLLKNCWNSLPVGGKVLIIEFVLPNELGNNAESFNALIPDLLLMALNPGGKERTISEYDDLGKAAGFIKTIPIPISNGLHVIEFHK

SEQ ID NO: 93 - protein, 365 aa, Fragaria sp.
MGSTGETQMTPTHVSDEEANLFAMQLASASVLPMVLKAAIELDLLEIMAKAGPGSFLSPSDLASQLPTKNPEAPVMLDRMLRLLASYSILTCSLRTLPDGKVERLYCLGPVCKFLTKNEDGVSIAALCLMNQDKVLVESWYHLKDAVLDGGIPFNKAYGMTAFDYHGTDPRFNKVFNKGMADHSTITMKKILETYKGFEGLKSIVDVGGGTGAVVNMIVSKYPSIKGINFDLPHVIEDAPQYPGVQHVGGDMFVSVPKGNAIFMKWICHDWSDEHCIKFLKNCYAALPDDGKVILAECILPVAPDTSLATKGVVHMDVIMLAHNPGGKERTEQEFEALAKGSGFQGIRVCCDAFNTYVIEFLKKI

SEQ ID NO: 94 - DNA, 23 bp, Sorghum bicolor
  source 1..23: mol_type = genomic DNA
cctcgcacagcgtagtccggtgc

SEQ ID NO: 95 - DNA, 23 bp, Sorghum bicolor
  source 1..23: mol_type = genomic DNA
attgaacacggtgttccacgagg

SEQ ID NO: 96 - DNA, 22 bp, Sorghum bicolor
  source 1..22: mol_type = genomic DNA
cctcgccagcgtagtccggtgc

SEQ ID NO: 97 - DNA, 24 bp, Sorghum bicolor
  source 1..24: mol_type = genomic DNA
attgaacacggtgttcccacgagg

SEQ ID NO: 98 - DNA, 18 bp, Sorghum bicolor
  source 1..18: mol_type = genomic DNA
cctcgcgtagtccggtgc

SEQ ID NO: 99 - DNA, 24 bp, Sorghum bicolor
  source 1..24: mol_type = genomic DNA
attgaacacggtgttcccacgagg

SEQ ID NO: 100 - DNA, 22 bp, Sorghum bicolor
  source 1..22: mol_type = genomic DNA
cctcgcaagcgtagtccggtgc

SEQ ID NO: 101 - DNA, 24 bp, Sorghum bicolor
  source 1..24: mol_type = genomic DNA
attgaacacggtgttcccacgagg

SEQ ID NO: 102 - protein, 371 aa, Sorghum bicolor
MGSYDSSSSSSNDSSARNEEDESCMFALKLLGGFAVPFTIKAVIELGVMDQLLTAERAMSAEELVAAAVAAQLPRPEVACTMVDRLLRFLASPASGARPRWWWARTTPPPPPAAAGATPRHPSASGSPGTASRIRCFRLGSTRHSWTAGKTLRMQCWKEQHHLRKPTGCQCSSTVQTDHTRCSHEAMANHSMIITKKLLKFFRGFEGLDVLVDVGGGNGTTLQMIRGQYKNMRGINYDLPHVIAQAAPVEGVEHVGGSMFDNIPRGNAVLLKWILHDWDDKACIKILKNCYTALHVRGKVIVLEYVVPDEPEPTLAAQGAFELDLTMLVTFGSGKERTQREFSELAMEAGFSREFKATYIFANVWALEFTK

SEQ ID NO: 103 - protein, 365 aa, Sorghum bicolor
MGSYDSSSSSSNDSSARNEEDESCMFALKLLGGFAVPFTIKAVIELGVMDQLLTAERAMSAEELVAAAVAAQLPRPEVACTMVDRLLRFLASRSPVHDRGGGGHGRRHHHHLLPPELRRVTRLQVVRQERRRGFGASAWDDDPKQDIPGQLAKHYGCSVGRSSTIENLRDANVRVPKYKRTIEHGVPTRQWQIIRLPRNCSSSSAASKALMCWSTAAATAPRCKLEVNIRIEATTTFLMSLRRLHQLKVWNMVAACSIIFHAEMQFCSSGFFMIGTTRRASRSRIAIQLSMEARSFWSTLFRMNQNLLLQLRVPSNWTSPCWSRLAVVKRGHRGSSPSSPWRPASLESLRLRISLPTSGPLSSQS

SEQ ID NO: 104 - protein, 371 aa, Sorghum bicolor
MGSYDSSSSSSNDSSARNEEDESCMFALKLLGGFAVPFTIKAVIELGVMDQLLTAERAMSAEELVAAAVAAQLPRPEVACTMVDRLLRFLASQASGARPRWWWARTTPPPPPAAAGATPRHPSASGSPGTASRIRCFRLGSTRHSWTAGKTLRMQCWKEQHHLRKPTGCQCSSTVQTDHTRCSHEAMANHSMIITKKLLKFFRGFEGLDVLVDVGGGNGTTLQMIRGQYKNMRGINYDLPHVIAQAAPVEGVEHVGGSMFDNIPRGNAVLLKWILHDWDDKACIKILKNCYTALHVRGKVIVLEYVVPDEPEPTLAAQGAFELDLTMLVTFGSGKERTQREFSELAMEAGFSREFKATYIFANVWALEFTK